(19) United States
(12) Patent Application Publication
(10) Pub. No.: US 2022/0380484 A1
(43) Pub. Date: Dec. 1, 2022

(54) ANTIBODIES FOR BINDING PLASMINOGEN

(71) Applicant: Monash University, Clayton, Victoria (AU)

(72) Inventors: James Whisstock, Clayton, Victoria (AU); Ruby Law, Clayton, Victoria (AU); Adam Quek, Clayton, Victoria (AU); Paul Conroy, Clayton, Victoria (AU); Dena Lyras, Clayton, Victoria (AU); Guojie Wu, Clayton, Victoria (AU)

(21) Appl. No.: 17/771,982

(22) PCT Filed: Oct. 28, 2020
(86) PCT No.: PCT/AU2020/051163

§ 371 Date: Apr. 26, 2022

(30) Foreign Application Priority Data
Oct. 28, 2019 (AU) 2019904052

(51) Int. Cl. C07K 16/40; A61P 7/04

(52) U.S. Cl. CPC C07K 16/40; A61P 7/04; A61K 2039/505

(57) ABSTRACT

The present invention relates to antigen binding proteins comprising an antigen binding domain that binds to plasminogen, wherein the antigen binding protein reduces the activation of plasminogen to plasmin, pharmaceutical compositions comprising the same, and methods and uses thereof.

### FIELD OF THE INVENTION

The invention relates to antigen binding proteins and related fragments thereof for binding to plasminogen, to production of said antigen binding proteins and fragments and to use of said antibodies and fragments for detection and therapy of various conditions.

### RELATED APPLICATION

This application claims priority from Australian provisional application AU 2019904052, the contents of which are hereby incorporated by reference in their entirety.

### BACKGROUND OF THE INVENTION

Plasminogen (PLG) is the inactive zymogen form of plasmin, a serine protease that has a broad specificity for target substrates that include fibrin, fibrinogen, complement components 3 and 5 (C3 and C5), vitronectin, osteocalcin, factors V, VIII and X and some collagenases. Thus, PLG and PLM together are involved in various important physiological and pathological processes including fibrinolysis and haemostasis, degradation of extracellular matrix, cell migration, embryonic development, tissue remodeling, inflammation, wound healing, angiogenesis and tumor invasion.

PLG is synthesized primarily in the liver, but also in major organs and tissues. Consequently, PLG is found in significant quantities in plasma and many extravascular fluids. Under physiological conditions, PLG is converted to the active form, plasmin (PLM), through cleavage in the activation loop. Activation can be mediated by urokinase plasminogen activator (uPA) or tissue plasminogen activator (tPA), or by various other proteases, and converts the single-chain PLG (amino acid residues 20-810) to PLM which consists of disulfide bond-linked heavy chain A (residues 20-580) and light chain B (residues 581-810). Heavy chain A contains 5 kringle domains (which mediate binding to substrates via lysine-binding regions) and light chain B corresponds to the serine protease domain. A fragment consisting of the first 4 kringle domains has been named as angiostatin, a novel angiogenesis inhibitor.

The plasminogen/plasmin system has been implicated in a variety of physiological and pathological processes such as fibrinolysis, tissue remodeling, cell migration, inflammation, and tumor invasion and metastasis. Hereditary defects of plasminogen are a predisposing risk factor for thromboembolic disease.

There is a need for compositions and methods for modulating the plasmin system for the treatment and/or prevention of various conditions.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides an antigen binding protein comprising an antigen binding domain that binds to plasminogen, wherein the antigen binding protein reduces the activation of plasminogen.

In any aspect, an antigen binding protein of the invention may reduce the activation of plasminogen by any one or more plasminogen activators. A plasminogen activator is any enzyme that can cleave the Arg₅₆₁-Val₅₆₂ bond (numbering as per human plasminogen). Exemplary plasminogen activators are plasminogen-cleaving serine proteases including the coagulation proteins factor IX, factor X, and prothrombin (factor II), protein C, chymotrypsin and trypsin, various leukocyte elastases, streptokinase (SK), staphylokinase, urokinase plasminogen activator (uPA), tissue plasminogen activator (tPA), and plasmin. Preferably, the plasminogen activator is selected from the group consisting of streptokinase (SK), urokinase plasminogen activator (uPA) and tissue plasminogen activator (tPA). Preferably the reduction in SK-, uPA- and/or tPA-mediated activation of plasminogen is in the presence of one or more ligands or substrates of plasminogen/plasmin. Preferably, the cofactors are selected from the group consisting of ε-aminocaproic acid (EACA) or other lysine analog, fibrinogen (Fg) and fibrin (Fn). Methods of measuring activation of plasminogen are known in the art and can be used to determine a reduction in activation of plasminogen by an antigen binding protein of the invention. Exemplary methods are described herein. A reduction in activation of plasminogen may be determined by measuring a reduction in the amount of active plasmin formed.

In any aspect, an antigen binding protein of the invention does not detectably inhibit or significantly inhibit the activity of plasmin. In further aspects, an antigen binding protein of the invention does not detectably bind to the active site (i.e. the catalytic triad) of plasmin. The binding of an antigen binding protein to plasmin may be determined by any method described herein.

In any aspect, an antigen binding protein of the invention binds to a kringle domain of plasminogen, preferably to kringle domain 5. In any aspect, an antigen binding protein of the invention binds to a kringle domain and the serine protease domain of plasminogen. In any aspect of the invention, the antigen binding protein binds to a kringle domain, activation loop and serine protease domain of plasminogen. In any aspect, an antigen binding domain of the invention binds only to a region of plasminogen that includes the activation loop and a kringle domain of plasminogen, preferably kringle 5 but does not include the serine protease domain. In other words, the antigen binding domain of the invention preferably does not bind to a region of plasminogen that does not include the activation loop. In any aspect, the antigen binding protein of the invention binds to the serine protease domain but does not bind to a kringle domain of plasminogen. In further aspects, the antigen binding protein of the invention also interacts with the serine protease domain.

In any aspect, an antigen binding protein of the invention binds to a region of plasminogen that comprises the activation loop of plasminogen. Preferably, the region comprising the activation loop comprises an amino acid sequence from between Arg₄₉₃ to His₅₆₉ of plasminogen (numbering as per human plasminogen provided in SEQ ID NO: 65), or comprises a sequence from between Lys₄₆₈ and His₅₆₉, more preferably wherein the activation loop comprises the sequence from Ala₅₄₃ to Arg₅₈₂ of plasminogen according to the numbering shown in SEQ ID NO: 65. Preferably, the antigen binding protein binds to a peptide comprising the sequence as set forth in SEQ ID NO: 66, or a fragment thereof. Preferably, the antigen binding protein of the invention binds to one or more of residues Arg₅₆₁ and Val₅₆₂ in the plasminogen loop of plasminogen.

In any aspect, an antigen binding protein of the invention binds to one or more residues of a kringle 5 domain of plasminogen at a position, or position equivalent to, Arg₄₉₃, Ser₄₉₅, Ile₄₉₆, Asp₅₁₆, Gly₅₁₇, AsP₅₁₈, Val₅₁₉, Tyr₅₂₅, and Tyr₅₃₃. Preferably, the antigen binding protein of the invention binds to one or more residues of a kringle 5 domain of plasminogen at a position, or position equivalent to, those shown in Table 2. Preferably, the residues of the antigen binding protein of the invention that bind to one or more residues of a kringle 5 domain of plasminogen are the residues defined, or are amino acid residues at a positions equivalent to those shown in Table 2.

In any aspect, an antigen binding protein of the invention binds to one or more residues of a kringle 5 domain of plasminogen at a position, or position equivalent to, Lys₄₆₈, Arg₄₉₃, Ser₄₉₅, Ile₄₉₆, Asp₅₁₆, Gly₅₁₇, and Tyr₅₂₅. Preferably, the antigen binding protein of the invention binds to one or more residues of a kringle 5 domain of plasminogen at a position, or position equivalent to, those shown in Table 3. Preferably, the residues of the antigen binding protein of the invention that bind to one or more residues of a kringle 5 domain of plasminogen are as defined, or are amino acid residues at a position equivalent to those shown in Table 3.

In any aspect, an antigen binding protein of the invention binds to one or more residues of a serine protease domain of plasminogen at a position, or position equivalent to, Glu₅₅₄, Lys₅₅₆, Lys₅₅₇, His₅₆₉, and Asp₇₅₁. Preferably, the antigen binding protein of the invention binds to one or more residues of a serine protease domain of plasminogen at a position, or position equivalent to, those shown in Table 2. Preferably, the residues of the antigen binding protein of the invention that bind to one or more residues of a serine protease domain of plasminogen are as defined, or are amino acid residues at a position equivalent to those shown in Table 2.

In any aspect, an antigen binding protein of the invention binds to one or more residues of a serine protease domain of plasminogen at a position, or position equivalent to, Glu₅₅₄, Lys₅₅₆, Lys₅₅₇, Arg₅₆₁, and His₅₆₉. Preferably, the antigen binding protein of the invention binds to one or more residues of a serine protease domain of plasminogen at a position, or position equivalent to, those shown in Table 3. Preferably, the residues of the antigen binding protein of the invention that bind to one or more residues of a serine protease domain of plasminogen are as defined, or are amino acid residues at a position equivalent to those shown in Table 3.

In any aspect, an antigen binding protein of the invention binds to one or more residues of a serine protease domain of plasminogen at a position, or position equivalent to, Asp₆₇₆, Arg₆₇₇, Arg₇₁₂, Glu₇₁₄ and Asn₇₆₉. Preferably, the antigen binding protein of the invention binds to one or more residues of a serine protease domain of plasminogen at a position, or position equivalent to, those shown in Table 4. Preferably, the residues of the antigen binding protein of the invention that bind to one or more residues of a serine protease domain of plasminogen are as defined, or are amino acid residues at a position equivalent to those shown in Table 4.

The present invention also provides an antigen binding protein (e.g., antibody) that has the same, or substantially the same amino acids at the positions, or equivalent positions to the residues specified for the G05 antibody in Tables 2 or 3.

The present invention also provides an antigen binding protein (e.g., antibody) that has the same, or substantially the same amino acids at the positions, or equivalent positions to the residues specified for the G11 antibody in Table 4.

The present invention also provides an antigen binding protein that binds to or specifically binds to plasminogen and wherein the antigen binding protein competitively inhibits binding of the G05 or G11 antibodies (i.e., comprising: a VH comprising a sequence set forth in SEQ ID NO: 8 and a VL comprising a sequence set forth in SEQ ID NO: 7; or a VH comprising a sequence set forth in SEQ ID NO: 40 and a VL comprising a sequence set forth in SEQ ID NO: 39) to plasminogen.

In any aspect of the present invention, the interaction of a residue of an antigen binding protein of the invention with a residue of plasminogen may be defined by x-ray crystallography and a contact distance analysis of 0 to 3.9 Å (inclusive).

The present invention also provides an antigen binding protein that binds to the same epitope on plasminogen as an antibody that comprises a VH domain comprising the amino acid sequence as set forth in SEQ ID NO: 8 wherein the antigen binding protein reduces or inhibits the activation of plasminogen.

The present invention also provides an antigen binding protein that binds to the same epitope on plasminogen as an antibody that comprises a VH domain comprising the amino acid sequence as set forth in SEQ ID NO: 8 and a VL domain comprising the amino acid sequence as set forth in SEQ ID NO: 7, wherein the antigen binding protein reduces or inhibits the activation of plasminogen. In one embodiment, the epitope is defined by x-ray crystallography. Preferably, the epitope is defined by x-ray crystallography and a contact distance analysis of 0 to 3.9 Å (inclusive). Preferably, the antigen binding protein covers a surface area of plasminogen of SEQ ID NO: 65 of about 1000 to 1300 Å².

The present invention also provides an antigen binding protein that binds to the same epitope on plasminogen as an antibody that comprises a VH domain comprising the amino acid sequence as set forth in SEQ ID NO: 40 and a VL domain comprising the amino acid sequence as set forth in SEQ ID NO: 39, wherein the antigen binding protein reduces or inhibits the activation of plasminogen. In one embodiment, the epitope is defined by x-ray crystallography. Preferably, the epitope is defined by x-ray crystallography and a contact distance analysis of 0 to 3.9 Å (inclusive).

In any aspect, an antigen binding protein of the invention may bind to plasminogen and exhibit a K_{D} of less than 15 nM, less than 10 nM or equal to about 8 nM or less. Preferably, the K_{D} is determined using any assay as described herein, for example surface plasmon resonance (SPR).

In any aspect, an antigen binding protein of the invention may bind to plasminogen and exhibit a kₐ (M⁻¹s⁻¹) of greater than about 1×10⁴, greater than about 1×10⁵, or greater than or equal to about 4×10⁵.

In any aspect, an antigen binding protein of the invention may bind to plasminogen and exhibit a kₐ(s⁻¹) of less than about 1×10⁻³, less than about 5×10⁻³, or less than or equal to about 2×10⁻³ or less than about 10×10⁻⁴. Preferably, the kₐ is about 7×10⁻⁴, or any value as described herein.

In any aspect of the invention, an antigen binding protein of the invention may inhibit streptokinase-, tPA- or uPA-mediated activation of plasminogen with an IC₅₀ of less than 500 nM, 300 nM, 250 nM, 200 nM, 150 nM, 100 nM, 80 nM, 60 nM or 50 nM. Preferably, the IC₅₀ is equal to about 18 nM or any value as described herein, when measured in solution in the presence of EACA. Alternatively, the IC₅₀ is equal to about 290 nM or any value as described herein, when measured in presence of fibrin.

An antigen binding protein of the invention may bind to a peptide derived from SEQ ID NO: 65. For example, the antigen binding protein of the invention may bind to a peptide consisting of 4, 5, 7, 8, 9, 10 or more contiguous amino acid residues of the sequence of SEQ ID NO: 65. In some embodiments, the antigen binding protein of the invention binds to a peptide comprising, consisting essentially of or consisting of residues of 554 to 569 of SEQ ID NO: 65. In some embodiments, the antigen binding protein of the invention binds to a peptide comprising, consisting essentially of or consisting of residues of 493-533 of SEQ ID NO: 65. In some embodiments, the antigen binding protein of the invention binds to a peptide comprising, consisting essentially of or consisting of residues of 468 to 525 of SEQ ID NO: 65. In some embodiments, the antigen binding protein of the invention binds to a peptide comprising, consisting essentially of or consisting of residues of 554 to 569 of SEQ ID NO: 65. In some embodiments, the antigen binding protein of the invention binds to a peptide comprising, consisting essentially of or consisting of residues of 554 to 569 of SEQ ID NO: 65. In some embodiments, the antigen binding protein of the invention binds to a peptide comprising, consisting essentially of or consisting of residues of 493 to 533 of SEQ ID NO: 65, and binds to a peptide comprising, consisting essentially of or consisting of residues of 554 to 569 of SEQ ID NO: 65. In some embodiments, the antigen binding protein of the invention binds to a peptide comprising, consisting essentially of or consisting of residues of 468 to 525 of SEQ ID NO: 65, and binds to a peptide comprising, consisting essentially of or consisting of residues of 554 to 569 of SEQ ID NO: 65. In still further embodiments, the antigen binding protein of the invention binds to a peptide comprising, consisting essentially of or consisting of residues of 468 to 525 of SEQ ID NO: 65, and binds to a peptide comprising, consisting essentially of or consisting of residues of 554 to 751 of SEQ ID NO: 65

In preferred embodiments, the antigen binding protein of the invention does not bind to a region of plasminogen or plasmin that only comprises the serine protease domain. In other words, the antigen binding protein of the invention is a protein that binds to a region of plasminogen that comprises the activation loop which exists between the region known as kringle 5 and the serine protease domain.

In further embodiments, the antigen binding protein of the invention does not detectably bind to a region of plasminogen that comprises the catalytic triad of the serpin protease domain. Accordingly, in certain embodiments, the antigen binding protein of the invention does not bind to a peptide comprising or consisting of the amino acid sequence 570 to 741 of SEQ ID NO: 65, preferably comprising or consisting of the amino acid sequence from His₆₀₃ to Ala₇₄₁ of SEQ ID NO: 65.

In any aspect, the antigen binding protein does not significantly reduce the activity of plasmin. Preferably, the antigen binding protein does not result in a significant reduction in activity of plasmin as measured using any assay described herein which has the capacity to determine plasmin activity.

In any aspect, the antigen binding protein does not significantly reduce the activity of any one or more of tPA, thrombin, trypsin, Factor Xa (FXa) and plasma kallikrein.

In certain embodiments, the CDRs of the antigen binding proteins may be determined using the IMGT domain gap numbering system.

The invention provides an antigen binding protein for binding to plasminogen, the antigen binding protein comprising:

FR1-CDR1-FR2-CDR2-FR3-CDR3-FR4, and

FR1a-CDR1a-FR2a-CDR2a-FR3a-CDR3a-FR4a,

wherein:

FR1, FR2, FR3 and FR4 are each framework regions;

CDR1, CDR2 and CDR3 are each complementarity determining regions;

FR1a, FR2a, FR3a and FR4a are each framework regions;

CDR1a, CDR2a and CDR3a are each complementarity determining regions;

wherein the sequence of any of the framework regions or complementarity determining regions are as described herein.

The invention provides an antigen binding protein for binding to plasminogen, the antigen binding protein comprising:

FR1-CDR1-FR2-CDR2-FR3-CDR3-FR4, and

FR1a-CDR1a-FR2a-CDR2a-FR3a-CDR3a-FR4a,

wherein:

FR1, FR2, FR3 and FR4 are each framework regions;

CDR1, CDR2 and CDR3 are each complementarity determining regions;

FR1a, FR2a, FR3a and FR4a are each framework regions;

CDR1a, CDR2a and CDR3a are each complementarity determining regions;

wherein the sequence of any of the complementarity determining regions have an amino acid sequence as described in Table 1 below. Preferably, the framework regions have an amino acid sequence also as described in Table 1 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

In one embodiment, the antigen binding protein comprises:

FR1-CDR1-FR2-CDR2-FR3-CDR3-FR4-linker-FR1a-CDR1a-FR2a-CDR2a-FR3a-CDR3a-FR4a.

As defined herein, the linker may be a chemical, one or more amino acids, or a disulphide bond formed between two cysteine residues.

The invention provides an antigen binding protein comprising, consisting essentially of or consisting of an amino acid sequence of (in order of N to C terminus or C to N terminus):
SEQ ID NO: 7 and 8; or
SEQ ID NO: 39 and 40.

The present invention also provides an antigen binding protein comprising an antigen binding domain of an antibody, wherein the antigen binding domain binds to or specifically binds to plasminogen, wherein the antigen binding domain comprises at least one of:

(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO:4, a CDR2 comprising a sequence at least about 80%, at least 85%, at least 90%, at least 92%, at least 95%, at least 97%, at least 99% identical to a sequence set in SEQ ID NO:5 and a CDR3 comprising a sequence at least about 80%, at least 85%, at least 90%, at least 92%, at least 95%, at least 97%, at least 99% identical to a sequence set forth in SEQ ID NO: 6;

(ii) a VH comprising a sequence at least about 95% or 96% or 97% or 98% or 99% identical to a sequence set forth in SEQ ID NO: 8;

(iii) a VL comprising a CDR1 comprising a sequence at least about 80%, at least 85%, at least 90%, at least 92%, at least 95%, at least 97%, at least 99% identical to a sequence set forth in SEQ ID NO: 1, a CDR2 comprising a sequence at least about 80%, at least 85%, at least 90%, at least 92%, at least 95%, at least 97%, at least 99% identical to a sequence set forth in SEQ ID NO: 2 and a CDR3 comprising a sequence at least about 80%, at least 85%, at least 90%, at least 92%, at least 95%, at least 97%, at least 99% identical to a sequence set forth in SEQ ID NO: 3;

(iv) a VL comprising a sequence at least about 95% identical to a sequence set forth in SEQ ID NO: 7;

(v) a VH comprising a CDR1 comprising a sequence set forth in SEQ ID NO: 4, a CDR2 comprising a sequence set forth between in SEQ ID NO: 5 and a CDR3 comprising a sequence set forth in SEQ ID NO: 6;

(vi) a VH comprising a sequence set forth in SEQ ID NO: 8;

(vii) a VL comprising a CDR1 comprising a sequence set SEQ ID NO: 1, a CDR2 comprising a sequence set forth in SEQ ID NO: 2 and a CDR3 comprising a sequence set forth in SEQ ID NO: 3;

(viii) a VL comprising a sequence set forth in SEQ ID NO: 7;

(ix) a VH comprising a CDR1 comprising a sequence set forth in SEQ ID NO: 4, a CDR2 comprising a sequence set forth between in SEQ ID NO: 5 and a CDR3 comprising a sequence set forth in SEQ ID NO: 6; and a VL comprising a CDR1 comprising a sequence set SEQ ID NO: 1, a CDR2 comprising a sequence set forth in SEQ ID NO: 2 and a CDR3 comprising a sequence set forth in SEQ ID NO: 3; or

(x) a VH comprising a sequence set forth in SEQ ID NO: 8 and a VL comprising a sequence set forth in SEQ ID NO: 7.

In any aspect of the invention, the antigen binding domain further comprises at least one of:

(i) a VH comprising a framework region (FR) 1 comprising a sequence at least about 80%, at least 85%, at least 90%, at least 92%, at least 95%, at least 97%, at least 99% identical to a sequence set forth in SEQ ID NO:21, a FR2 comprising a sequence at least about 80%, at least 85%, at least 90%, at least 92%, at least 95%, at least 97%, at least 99% identical to a sequence set in SEQ ID NO:22, a FR3 comprising a sequence at least about 80%, at least 85%, at least 90%, at least 92%, at least 95%, at least 97%, at least 99% identical to a sequence set forth in SEQ ID NO: 23, and a FR4 comprising a sequence at least about 80%, at least 85%, at least 90%, at least 92%, at least 95%, at least 97%, at least 99% identical to a sequence set forth in SEQ ID NO: 24;

(ii) a VL comprising a FR1 comprising a sequence at least about 80%, at least 85%, at least 90%, at least 92%, at least 95%, at least 97%, at least 99% identical to a sequence set forth in SEQ ID NO: 17, a FR2 comprising a sequence at least about 80%, at least 85%, at least 90%, at least 92%, at least 95%, at least 97%, at least 99% identical to a sequence set forth in SEQ ID NO: 18, a FR3 comprising a sequence at least about 80%, at least 85%, at least 90%, at least 92%, at least 95%, at least 97%, at least 99% identical to a sequence set forth in SEQ ID NO: 19, and a FR4 comprising a sequence at least about 80%, at least 85%, at least 90%, at least 92%, at least 95%, at least 97%, at least 99% identical to a sequence set forth in SEQ ID NO: 20;

(iii) a VH comprising a FR1 comprising a sequence set forth in SEQ ID NO: 21, a FR2 comprising a sequence set forth between in SEQ ID NO: 22, a FR3 comprising a sequence set forth in SEQ ID NO: 23, and a FR4 comprising a sequence set forth in SEQ ID NO: 24;

(iv) a VL comprising a FR1 comprising a sequence set forth in SEQ ID NO: 17, a FR2 comprising a sequence set forth between in SEQ ID NO: 18, a FR3 comprising a sequence set forth in SEQ ID NO: 19, and a FR4 comprising a sequence set forth in SEQ ID NO: 20; or

(v) a VH comprising a FR1 comprising a sequence set forth in SEQ ID NO: 21, a FR2 comprising a sequence set forth between in SEQ ID NO: 22, a FR3 comprising a sequence set forth in SEQ ID NO: 23, and a FR4 comprising a sequence set forth in SEQ ID NO: 24; and a VL comprising a FR1 comprising a sequence set forth in SEQ ID NO: 17, a FR2 comprising a sequence set forth between in SEQ ID NO: 18, a FR3 comprising a sequence set forth in SEQ ID NO: 19, and a FR4 comprising a sequence set forth in SEQ ID NO: 20.

The present invention also provides an antigen binding protein comprising an antigen binding domain of an antibody, wherein the antigen binding domain binds to or specifically binds to plasminogen, wherein the antigen binding domain comprises at least one of:

(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 36, a CDR2 comprising a sequence at least about 80%, at least 85%, at least 90%, at least 92%, at least 95%, at least 97%, at least 99% identical to a sequence set in SEQ ID NO: 37 and a CDR3 comprising a sequence at least about 80%, at least 85%, at least 90%, at least 92%, at least 95%, at least 97%, at least 99% identical to a sequence set forth in SEQ ID NO: 38;

(ii) a VH comprising a sequence at least about 95% or 96% or 97% or 98% or 99% identical to a sequence set forth in SEQ ID NO: 40;

(iii) a VL comprising a CDR1 comprising a sequence at least about 80%, at least 85%, at least 90%, at least 92%, at least 95%, at least 97%, at least 99% identical to a sequence set forth in SEQ ID NO: 33, a CDR2 comprising a sequence at least about 80%, at least 85%, at least 90%, at least 92%, at least 95%, at least 97%, at least 99% identical to a sequence set forth in SEQ ID NO: 34 and a CDR3 comprising a sequence at least about 80%, at least 85%, at least 90%, at least 92%, at least 95%, at least 97%, at least 99% identical to a sequence set forth in SEQ ID NO: 35;

(iv) a VL comprising a sequence at least about 95% identical to a sequence set forth in SEQ ID NO: 39;

(v) a VH comprising a CDR1 comprising a sequence set forth in SEQ ID NO: 36, a CDR2 comprising a sequence set forth between in SEQ ID NO: 37 and a CDR3 comprising a sequence set forth in SEQ ID NO: 38;

(vi) a VH comprising a sequence set forth in SEQ ID NO: 40;

(vii) a VL comprising a CDR1 comprising a sequence set SEQ ID NO: 33, a CDR2 comprising a sequence set forth in SEQ ID NO: 34 and a CDR3 comprising a sequence set forth in SEQ ID NO: 35;

(viii) a VL comprising a sequence set forth in SEQ ID NO: 39;

(ix) a VH comprising a CDR1 comprising a sequence set forth in SEQ ID NO: 36, a CDR2 comprising a sequence set forth between in SEQ ID NO: 37 and a CDR3 comprising a sequence set forth in SEQ ID NO: 38; and a VL comprising a CDR1 comprising a sequence set SEQ ID NO: 33, a CDR2 comprising a sequence set forth in SEQ ID NO: 34 and a CDR3 comprising a sequence set forth in SEQ ID NO: 35; or

(x) a VH comprising a sequence set forth in SEQ ID NO: 40 and a VL comprising a sequence set forth in SEQ ID NO: 39.

In any aspect of the invention, the antigen binding domain further comprises at least one of:

(i) a VH comprising a framework region (FR) 1 comprising a sequence at least about 80%, at least 85%, at least 90%, at least 92%, at least 95%, at least 97%, at least 99% identical to a sequence set forth in SEQ ID NO: 53, a FR2 comprising a sequence at least about 80%, at least 85%, at least 90%, at least 92%, at least 95%, at least 97%, at least 99% identical to a sequence set in SEQ ID NO: 54, a FR3 comprising a sequence at least about 80%, at least 85%, at least 90%, at least 92%, at least 95%, at least 97%, at least 99% identical to a sequence set forth in SEQ ID NO: 55, and a FR4 comprising a sequence at least about 80%, at least 85%, at least 90%, at least 92%, at least 95%, at least 97%, at least 99% identical to a sequence set forth in SEQ ID NO: 56;

(ii) a VL comprising a FR1 comprising a sequence at least about 80%, at least 85%, at least 90%, at least 92%, at least 95%, at least 97%, at least 99% identical to a sequence set forth in SEQ ID NO: 49, a FR2 comprising a sequence at least about 80%, at least 85%, at least 90%, at least 92%, at least 95%, at least 97%, at least 99% identical to a sequence set forth in SEQ ID NO: 50, a FR3 comprising a sequence at least about 80%, at least 85%, at least 90%, at least 92%, at least 95%, at least 97%, at least 99% identical to a sequence set forth in SEQ ID NO: 51, and a FR4 comprising a sequence at least about 80%, at least 85%, at least 90%, at least 92%, at least 95%, at least 97%, at least 99% identical to a sequence set forth in SEQ ID NO: 52;

(iii) a VH comprising a FR1 comprising a sequence set forth in SEQ ID NO: 53, a FR2 comprising a sequence set forth between in SEQ ID NO: 54, a FR3 comprising a sequence set forth in SEQ ID NO: 55, and a FR4 comprising a sequence set forth in SEQ ID NO: 56;

(iv) a VL comprising a FR1 comprising a sequence set forth in SEQ ID NO: 49, a FR2 comprising a sequence set forth between in SEQ ID NO: 50, a FR3 comprising a sequence set forth in SEQ ID NO: 51, and a FR4 comprising a sequence set forth in SEQ ID NO: 52; or

(v) a VH comprising a FR1 comprising a sequence set forth in SEQ ID NO: 53, a FR2 comprising a sequence set forth between in SEQ ID NO: 54, a FR3 comprising a sequence set forth in SEQ ID NO: 55, and a FR4 comprising a sequence set forth in SEQ ID NO: 56; and a VL comprising a FR1 comprising a sequence set forth in SEQ ID NO: 49, a FR2 comprising a sequence set forth between in SEQ ID NO: 50, a FR3 comprising a sequence set forth in SEQ ID NO: 51, and a FR4 comprising a sequence set forth in SEQ ID NO: 52.

As described herein, the antigen binding protein may be in the form of:

(i) a single chain Fv fragment (scFv);

(ii) a dimeric scFv (di-scFv);

(iii) one of (i) or (ii) linked to a constant region of an antibody, Fc or a heavy chain constant domain (CH) 2 and/or CH3; or

(iv) one of (i) or (ii) linked to a protein that binds to an immune effector cell.

Further, as described herein, the antigen binding protein may be in the form of:

(i) a diabody;

(ii) a triabody;

(iii) a tetrabody;

(iv) a Fab;

(v) a F(ab′)2;

(vi) a Fv;

(vii) a bispecific antibody or other form of multispecific antibody;

(viii) one of (i) to (vii) linked to a constant region of an antibody, Fc or a heavy chain constant domain (CH) 2 and/or CH3; or

(viv) one of (i) to (vii) linked to a protein that binds to an immune effector cell.

The foregoing antigen binding proteins can also be referred to as antigen binding domains of antibodies.

Preferably, an antigen binding protein as described herein is an antibody or antigen binding fragment thereof. Typically, the antigen binding protein is an antibody, for example, a monoclonal antibody. The antigen binding protein may be in the form of a recombinant or modified antibody (e.g., chimeric antibody, humanized antibody, human antibody, CDR-grafted antibody, primatized antibody, de-immunized antibody, synhumanized antibody, half-antibody, bispecific antibody, trispecific antibody or multispecific antibody). The antibody may further comprise a chemical modification, such as conjugation to an active agent or radiolabel, or an agent for improving solubility or other modification described herein.

As used herein the antigen binding protein may be a variable domain.

The present invention also provides a plasminogen antibody comprising a light chain variable region and a heavy chain variable region,

wherein said light chain variable region comprises:

a CDR L1 as set forth in SEQ ID NO: 1, a CDR L2 as set forth in SEQ ID NO: 2 and a CDR L3 as set forth in SEQ ID NO: 3; and

wherein said heavy chain variable region comprises:

a CDR H1 as set forth in SEQ ID NO: 4, a CDR H2 as set forth in SEQ ID NO: 5, and a CDR H3 as set forth in SEQ ID NO: 6.

In any aspect of the invention, a plasminogen antibody comprises a light chain variable region that comprises the sequence of SEQ ID NO: 7.

In any aspect of the invention, a plasminogen antibody comprises a heavy chain variable region that comprises the sequence of SEQ ID NO: 8.

In any aspect of the invention, a plasminogen antibody comprises a light chain variable region that comprises a FR L1 as set forth in SEQ ID NO: 17, FR L2 as set forth in SEQ ID NO: 18, a FR L3 as set forth in SEQ ID NO: 19 and a FR L4 as set forth in SEQ ID NO: 20.

In any aspect of the invention, a plasminogen antibody comprises a heavy chain variable region that comprises a FR H1 as set forth in SEQ ID NO: 21, FR H2 as set forth in SEQ ID NO: 22, a FR H3 as set forth in SEQ ID NO: 23 and a FR H4 as set forth in SEQ ID NO: 24.

The present invention also provides a plasminogen antibody comprising a light chain variable region and a heavy chain variable region, wherein said light chain variable region comprises:

a CDR L1 as set forth in SEQ ID NO: 33, a CDR L2 as set forth in SEQ ID NO: 34 and a CDR L3 as set forth in SEQ ID NO: 35; and

wherein said heavy chain variable region comprises:

a CDR H1 as set forth in SEQ ID NO: 36, a CDR H2 as set forth in SEQ ID NO: 37, and a CDR H3 as set forth in SEQ ID NO: 38.

In any aspect of the invention, a plasminogen antibody comprises a light chain variable region that comprises the sequence of SEQ ID NO: 39.

In any aspect of the invention, a plasminogen antibody comprises a heavy chain variable region that comprises the sequence of SEQ ID NO: 40.

In any aspect of the invention, a plasminogen antibody comprises a light chain variable region that comprises a FR L1 as set forth in SEQ ID NO: 49, FR L2 as set forth in SEQ ID NO: 50, a FR L3 as set forth in SEQ ID NO: 51 and a FR L4 as set forth in SEQ ID NO: 52.

In any aspect of the invention, a plasminogen antibody comprises a heavy chain variable region that comprises a FR H1 as set forth in SEQ ID NO: 53, FR H2 as set forth in SEQ ID NO: 54, a FR H3 as set forth in SEQ ID NO: 55 and a FR H4 as set forth in SEQ ID NO: 56.

In any aspect or embodiment, the antibody is a naked antibody. Specifically, the antibody is in a non-conjugated form and is not adapted to form a conjugate.

In certain embodiments, the complementarity determining region sequences (CDRs) of an antigen binding protein of the invention may be defined according to the IMGT numbering system.

Reference herein to a protein or antibody that “binds to” plasminogen provides literal support for a protein or antibody that “binds specifically to” or “specifically binds to” plasminogen.

The present invention also provides antigen binding domains or antigen binding fragments of the foregoing antibodies.

The invention also provides a fusion protein comprising an antigen binding protein, immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody as described herein.

The invention also provides a conjugate in the form of an antigen binding protein, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody or fusion protein as described herein conjugated to a label or a cytotoxic agent.

The invention also provides an antibody for binding to an antigen binding protein, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein, or conjugate as described herein.

The invention also provides a nucleic acid encoding an antigen binding protein, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

In one example, such a nucleic acid is included in an expression construct in which the nucleic acid is operably linked to a promoter. Such an expression construct can be in a vector, e.g., a plasmid.

In examples of the invention directed to single polypeptide chain antigen binding protein, the expression construct may comprise a promoter linked to a nucleic acid encoding that polypeptide chain.

In examples directed to multiple polypeptide chains that form an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:

(i) a promoter

(ii) a nucleic acid encoding a first polypeptide;

(iii) an internal ribosome entry site; and

(iv) a nucleic acid encoding a second polypeptide,

wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:

(i) a first expression construct comprising a nucleic acid encoding a polypeptide comprising a VH operably linked to a promoter; and

(ii) a second expression construct comprising a nucleic acid encoding a polypeptide comprising a VL operably linked to a promoter.

The invention provides a cell comprising a vector or nucleic acid described herein. Preferably, the cell is isolated, substantially purified or recombinant. In one example, the cell comprises the expression construct of the invention or:

(i) a first expression construct comprising a nucleic acid encoding a polypeptide comprising a VH operably linked to a promoter; and

(ii) a second expression construct comprising a nucleic acid encoding a polypeptide comprising a VL operably linked to a promoter,

wherein the first and second polypeptides associate to form an antigen binding protein of the present invention.

Examples of cells of the present invention include bacterial cells, yeast cells, insect cells or mammalian cells.

The present invention also provides a peptide comprising, consisting essentially of or consisting of residues of 554 to 569 of SEQ ID NO: 65, binds to a peptide comprising, consisting essentially of or consisting of residues of 493 to 533, and binds to a peptide comprising, consisting essentially of or consisting of residues of 468 to 525 of SEQ ID NO: 65.

The invention also provides a pharmaceutical composition comprising an antigen binding protein, or comprising a CDR and/or FR sequence as described herein, or an immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody, fusion protein, or conjugate as described herein and a pharmaceutically acceptable carrier, diluent or excipient.

The invention also provides a diagnostic composition comprising an antigen binding protein, or comprising a CDR and/or FR sequence as described herein, or antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein, a diluent and optionally a label.

The invention also provides a kit or article of manufacture comprising an antigen binding protein, or comprising a CDR and/or FR sequence as described herein or an immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

An antigen binding protein as described herein may comprise a human constant region, e.g., an IgG constant region, such as an IgG1, IgG2, IgG3 or IgG4 constant region or mixtures thereof. In the case of an antibody or protein comprising a VH and a VL, the VH can be linked to a heavy chain constant region and the VL can be linked to a light chain constant region.

In one example, an antigen binding protein as described herein comprises a constant region of an IgG4 antibody or a stabilized constant region of an IgG4 antibody. In one example, the protein or antibody comprises an IgG4 constant region with a proline at position 241 (according to the numbering system of Kabat (Kabat et al., Sequences of Proteins of Immunological Interest Washington D.C. United States Department of Health and Human Services, 1987 and/or 1991)).

In one example, an antigen binding protein as described herein or a composition of an antigen binding protein as described herein, comprises a heavy chain constant region, comprising a stabilized heavy chain constant region, comprising a mixture of sequences fully or partially with or without the C-terminal lysine residue.

In one example, an antigen binding protein comprises a VH disclosed herein linked or fused to an IgG4 constant region or stabilized IgG4 constant region (e.g., as discussed above) and the VL is linked to or fused to a kappa light chain constant region.

The functional characteristics of an antigen binding protein of the invention will be taken to apply mutatis mutandis to an antibody of the invention.

An antigen binding protein as described herein may be purified, substantially purified, isolated and/or recombinant.

An antigen binding protein of the invention may be part of a supernatant taken from media in which a hybridoma expressing an antigen binding protein of the invention has been grown.

The present invention also provides a method of restoring haemostasis or for inhibiting plasminogen activation in a subject who has suffered a trauma or who is has suffered a haemorrhage or is haemorrhaging (for example, due to surgery, trauma or following child-birth), the method comprising administering an antigen binding protein of the invention to the subject, thereby restoring haemostasis or for inhibiting plasminogen activation in the subject.

The present invention provides a method for inhibiting fibrinolysis in a subject in need thereof, the method comprising administering an antigen binding protein of the invention to the subject, thereby inhibiting fibrinolysis in the subject. As used herein, a subject in need of inhibition of fibrinolysis may include a subject who has suffered a trauma (e.g., car accident, fall or other significant injury), has undergone surgery, is experiencing haemorrhage due to child-birth or for any other reason in which inhibition of fibrinolysis is required.

The present invention also provides a method of treating or preventing, or reducing the severity or progression of a bacterial infection in a subject, the method comprising administering an antigen binding protein of the invention to the subject, thereby treating or preventing, or reducing the severity or progression of the bacterial infection in the subject. In this regard, an antigen binding protein can be used to prevent a relapse of an infection, and this is considered preventing the infection.

The invention also provides a method for treating a condition associated with, or caused by, a bacterial in a subject, the method comprising administering to the subject an effective amount of the antigen binding protein of the invention, thereby treating the condition associated with, or caused by, a bacterial infection in the subject. A condition associated with, or caused by, a bacterial infection may be any condition described herein. In any embodiment, the bacterial infection may be chronic or acute.

The present invention also provides a method of reducing the severity of a bacterial infection in a subject, the method the method comprising administering an antigen binding protein of the invention to the subject, thereby reducing the severity of the bacterial infection in the subject.

Still further, the invention provides for a method of treating or preventing a cancer in a subject, the method comprising administering an antigen binding protein of the invention to the subject, thereby treating or preventing a cancer in the subject. As used herein, methods of treating cancer include methods of inhibiting, preventing or minimising spread or progression of a cancer, including inhibiting or preventing metastasis of cancer.

In any method of treatment of the invention, the method may comprise administration of a combination of any of the antigen binding proteins of the invention. In certain embodiments, the combination comprises a first antigen binding protein having a light chain variable region that comprises the sequence of SEQ ID NO: 39 and a heavy chain variable region that comprises the sequence of SEQ ID NO: 40; and a second antigen binding protein having a light chain variable region that comprises the sequence of SEQ ID NO: 7; and a heavy chain variable region that comprises the sequence of SEQ ID NO: 8.

The present invention also provides for the use of a plasminogen-binding antigen binding protein of the invention, in the manufacture of a medicament for the restoration of haemostasis or inhibition of excessive plasminogen activation in a subject who has suffered a trauma, or requires restoration of haemostasis or inhibition of plasminogen activation following surgery or childbirth.

The present invention also provides for the use of a plasminogen-binding antigen binding protein of the invention, in the manufacture of a medicament for the restoration or haemostasis or inhibition of excessive plasminogen activation in a subject who has suffered a trauma.

The present invention provides for the use of an antigen binding protein of the invention in the manufacture of a medicament for inhibiting fibrinolysis in a subject in need thereof. As used herein, a subject in need of inhibition of fibrinolysis may include a subject who has suffered a trauma (e.g., car accident, fall or other significant injury), has undergone surgery, is experiencing haemorrhage due to child-birth or for any other reason in which inhibition of fibrinolysis is required.

The present invention also provides for the use of a plasminogen-binding antigen binding protein of the invention, in the manufacture of a medicament for the treatment or prevention of a bacterial infection.

The present invention also provides for the use of a plasminogen-binding antigen binding protein of the invention, in the manufacture of a medicament for the treatment, prevention or reduction in severity of any condition or disease that is caused by or associated with a bacterial infection.

Still further, the invention provides for use of an antigen binding protein of the invention in the manufacture of a medicament for treating or preventing a cancer in a subject. The medicament may also be for inhibiting, preventing or minimising spread or progression of a cancer, including metastasis of a cancer.

In any use of the invention, the use comprises a combination of any of the antigen binding proteins of the invention in the manufacture of a medicament, preferably wherein the combination comprises a first protein having a light chain variable region that comprises the sequence of SEQ ID NO: 39 and a heavy chain variable region that comprises the sequence of SEQ ID NO: 40; and a second protein having a light chain variable region that comprises the sequence of SEQ ID NO: 7; and a heavy chain variable region that comprises the sequence of SEQ ID NO: 8.

The invention also provides for a pharmaceutical composition comprising a plasminogen-binding antigen binding protein of the invention, and a pharmaceutically acceptable excipient.

The pharmaceutical composition is preferably for a use as recited herein. Accordingly the invention provides a pharmaceutical composition comprising a plasminogen-binding antigen binding protein of the invention, for use in the restoration of haemostasis or inhibition of excessive plasminogen activation in a subject who has suffered a trauma, or requires restoration of haemostasis or inhibition of plasminogen activation following surgery or childbirth.

The present invention also provides a pharmaceutical composition comprising a plasminogen-binding antigen binding protein of the invention, for use in the restoration or haemostasis or inhibition of excessive plasminogen activation in a subject who has suffered a trauma.

The present invention provides for a pharmaceutical composition comprising an antigen binding protein of the invention for use in inhibiting fibrinolysis in a subject in need thereof. As used herein, a subject in need of inhibition of fibrinolysis may include a subject who has suffered a trauma (e.g., car accident, fall or other significant injury), has undergone surgery, is experiencing haemorrhage due to child-birth or for any other reason in which inhibition of fibrinolysis is required.

The present invention also provides a pharmaceutical composition comprising a plasminogen-binding antigen binding protein of the invention for use in the treatment or prevention of a bacterial infection.

The present invention also provides a pharmaceutical composition comprising a plasminogen-binding antigen binding protein of the invention for use in the treatment, prevention or reduction in severity of any condition or disease that is caused by or associated with a bacterial infection.

Further, the invention provides for a pharmaceutical composition comprising a plasminogen-binding antigen binding protein of the invention for use in treating or preventing a cancer in a subject. The pharmaceutical composition may also be for inhibiting, preventing or minimising spread or progression of a cancer, including metastasis of a cancer.

In any of the above recited methods or uses of the present invention, the bacterial infection may be caused by sporulating or non-sporulating bacteria. Where the infection is caused by sporulating bacteria, the infection may be characterised by bacteria in the vegetative state, or alternatively, bacteria in spore form. It will be understood that in targeting the spore form of the bacteria, the antigen binding proteins of the present invention can thereby act to prevent or reduce the likelihood of reinfection with the bacteria.

In any aspect of the present invention, the bacteria are gram-positive, preferably gram-positive cocci. In any aspect, the gram-positive bacteria are from the order Bacillales, Clostridiales or Lactobacillales.

In any aspect of the invention, the bacteria are from the family Streptococcaceae. Preferably the bacteria are from the genus Streptococcus. More preferably, the bacteria are Group A streptococcus (GAS), preferably Streptococcus pyogenes. In certain embodiments, the infection may be an infection caused by the bacteria selected from the group consisting of: Streptococcus pyogenes, Streptococcus dysgalactiae, Streptococcus pneumonia, Streptococcus agalactiae, Streptococcus canis, Streptococcus equisimilis, Streptococcus mutans, and Streptococcus suis.

In any aspect of the invention, the bacteria are from the family Bacillaceae, preferably from the genus Bacillus, more preferably wherein the infection is with a bacterium of the species Bacillus anthracis, and Bacillus cereus.

In any aspect of the invention, the bacteria are from the family Staphylococcaceae. Preferably the bacteria are from the genus Staphylococcus. More preferably, the infection may be an infection caused by the bacteria selected from the group consisting of: Staphylococcus aureus, including Methicillin-resistant Staphylococcus aureus (MRSA), Vancomycin-intermediate Staphylococcus aureus (VISA), and Vancomycin-resistant Staphylococcus aureus (VRSA). In certain aspects, the bacteria is Staphylococcus epidermidis.

In any aspect of the invention, the bacteria are from the family Peptostreptococcaceae. Preferably the bacteria are from the genus Clostridioides. More preferably, the bacterial infection is an infection by a bacterium selected from the group consisting of: Clostridium difficile (also known as Clostridioides difficile).

In any aspect, the bacteria are from the family Clostridiaceae. The bacteria may be from the genus Clostridium, optionally wherein the bacterial infection is an infection with any one of Clostridium botulinum, Clostridium tetani, Clostridium perfringens, and Clostridium sordellii.

In any aspect, the bacteria are from the family Porphyromonadaceae, including Porphyromonas gingivalis.

In any aspect of the invention, the bacteria are gram-negative, optionally from the order Enterobacteriales, Campylobacterales or Bacteroidales.

In any aspect of the invention, the bacteria are from the family Yersiniaceae, preferably from the genus Yersinia, more preferably wherein the infection is with a bacterium of the species Yersinia pestis, Yersinia enterocolitica.

In any aspect of the invention, the bacteria are from the family Helicobacteraceae, preferably from the genus Helicobacter, more preferably wherein the infection is with a bacterium of the species Helicobacter pylori.

Accordingly, a method, use or pharmaceutical composition of the invention is useful in the treatment, prevention or reduction in severity of any disease that is caused by or associated with a bacteria referred to herein. For example, a method, use or pharmaceutical composition of the invention may be for treating, preventing or reducing the severity of any disease/infection caused by or associated with a Group A Streptococcus (GAS), Staphylococcus or Bacillus sp. including, but not limited to, pharyngitis, tonsillitis, scarlet fever, cellulitis, erysipelas, rheumatic fever, skin and soft-tissue infection, endocarditis, bone and joint infections, infected implants, post-streptococcal glomerulonephritis, necrotizing fasciitis, myonecrosis subperiosteal abscesses, necrotizing pneumonia, pyomyositis, mediastinitis, myocardial, perinephric, hepatic, and splenic abscesses, septic thrombophlebitis, and severe ocular infections, including endophthalmitis and lymphangitis.

The methods, uses or pharmaceutical compositions of the invention may also be useful for treating, preventing or reducing the severity of any disease/infection caused by or associated with Clostroides (Clostridium) difficile including: gastrointestinal infection, mild-moderate diarrhoea and colitis.

The methods, uses or pharmaceutical compositions of the invention may also be useful for treating, preventing or reducing the severity of any disease/infection caused by or associated with P. gingivalis difficile including: gum disease and gum infection.

The methods, uses or pharmaceutical compositions of the invention may also be useful for treating, preventing or reducing the severity of any disease/infection caused by or associated with Yersinia sp. including: plague (pneumonic, septicemic or bubonic plague) and yersiniosis.

The methods, uses or pharmaceutical compositions of the invention may also be useful for treating, preventing or reducing the severity of any disease/infection caused by or associated with Helicobacter, including: acute or chronic gastritis, gastric ulcers, atrophy of stomach lining, susceptibility or risk for duodenal ulcers and stomach cancer.

In any embodiment of the invention, a method or use for treating or preventing bacterial infection includes a method or use for inhibiting or minimising the extent of invasion of host tissues/organs by the bacteria.

The methods and uses of the invention are applicable to the treatment or prevention of a cancer. Exemplary cancers include haematologic cancers, cancers of epithelial origin, liver cancer, pancreatic cancer, gastric cancer, osteosarcoma, endometrial cancer and ovarian cancer.

The present invention further provides a nucleic acid molecule encoding an antigen binding protein of the invention, or functional fragment or derivative thereof.

The invention also provides a cell comprising a vector or nucleic acid molecule described herein.

The invention also provides an animal or tissue derived therefrom comprising a cell described herein.

In another aspect the present invention provides a kit or article of manufacture including an antigen binding protein of the invention or pharmaceutical composition of the invention as described herein.

In other embodiments there is provided a kit for use in a therapeutic or prophylactic application mentioned herein, the kit including:
a container holding an antigen binding protein or pharmaceutical composition of the invention; and
a label or package insert with instructions for use.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Binding of G05 to plasminogen and plasmin. G05 was immobilised on a NiHc chip; binding kinetics of (A) Plasminogen and (B) Plasmin was measured by multi-cycle surface plasmon resonance (SPR). Dotted lines represent 1:1 Langmuir model fit to experimental data. (C) Table for kinetic (kₐ and k_{d}) and affinity constants (K_{D}).

FIG. 2: Binding of G11 to plasminogen and plasmin. G11 was immobilized on a NiHc chip; binding kinetics of (A) Plasminogen and (B) Plasmin was measured by multi-cycle surface plasmon resonance (SPR). Dotted lines represent 1:1 Langmuir model fit to experimental data. (C) Table for kinetic (kₐ and k_{d}) and affinity constants (K_{D}).

FIG. 3: Inhibition of tPA-mediated plasminogen activation by G05 (A) and G11 (B), in solution, in the presence of EACA.

FIG. 4: Inhibition of tPA-mediated plasminogen activation by G05 (A) and G11 (B), in the presence of fibrin.

FIG. 5: G05 does not inhibit plasmin activity; G11 moderately inhibits plasmin activity. Plasmin activity measured in the presence of G05 or G11 (0-2000 nM); 5 nM plasmin.

FIG. 6: Inhibition of streptokinase binding to plasminogen by G05. (A) 10 nM plasminogen_{R561A,s741A} was passed over streptokinase immobilized on a CM4 chip in the presence of G05 or a naïve chicken AB, gAb (at 0-500 nM). G05 showed inhibition especially at high concentrations. Naïve chicken antibody gAb, the control, showed no inhibition of Plg binding to SK. (B) Percentage of SK binding in the presence of 500 nM Abs, normalized against no antibody control.

FIG. 7: Inhibition of streptokinase-mediated plasminogen activation by G05. G05 inhibits plasminogen activation by SK as early addition of the antibody at t=0 and 10 minutes completely abolished plasmin generation.

FIG. 8: G05 and G11 binding to recombinant Serine Protease domain. (A) Size exclusion chromatography showing binding of G05 to a recombinant Kringle 5-serine protease domain (KSSP). A higher molecular weight complex was observed (KSSP+G05, solid line) compared to KSSP alone (dashed line) or G05 alone (dotted line). (B) Size exclusion chromatography showing binding of G11 to a recombinant serine protease domain (SP). Size exclusion chromatography shows higher molecular weight complex (SP+G11, solid line) compared to SP alone (dashed line) or G11 alone (dotted line).

FIG. 9: Crystal structures of G05 and G11 bound to Serine Protease domains of plasminogen. (A) The crystal structure of KR5-SP/G05 binary complex shows that G05 binds to the plasminogen activation loop. KR5 and SP are shown, activation loop in sticks, G05 light chain (LC) in and heavy chain (HC) labelled. At the top is the cartoon representation of the binary complex and the bottom shows the key residues, labelled and numbered, involved in intermolecular interactions as above. Dashed lines are used to illustrate polar interactions. (B) The crystal structure of SP/G11 binary complex shows that G11 forms polar interactions with the serine protease domain of plasminogen/plasmin such that it prevents plasmin generation and activity via distorting the catalytic pocket and surrounding loops. SP is shown, G11 light chain (LC) and heavy chain (HC) labelled. At the top is the cartoon representation of the binary complex and the bottom shows the key residues, labelled and numbered, involved in intermolecular interactions as above. Dashed lines are used to illustrate polar interactions.

FIG. 10: G05 Inhibition of plasminogen binding to Group A Streptococcus (GAS) measured by flow cytometry. Binding of fluorescently-labelled plasminogen to GAS is inhibited by G05 in comparison to gAb and controls. Also shown is inhibition of plasminogen binding to GAS by Tranexamic acid (TXA). Bar chart showing median fluorescence intensity plotted against the respective scFvs and controls.

FIG. 11: G05 inhibits plasminogen binding to Group A Streptococcus (GAS) measured by SDS-PAGE. Binding of fluorescently-labelled plasminogen to GAS was measured by (A) Fluorescence scan of SDS PAGE gel, showing total plasminogen added to the assay (Total), bound, unbound and wash collected during the assay. (B) The band intensity of the plasminogen on (A) was normalized against HBS control. Of all samples, only G05 showed significant inhibition of Plg binding to GAS cells.

FIG. 12: G05 inhibits plasminogen activation by Group A Streptococcus (GAS) (enzyme assay). (Top) Plasminogen bound to G05 was incubated with GAS. Plasmin generated was measured with the flourogenic substrate. Activation of plasminogen by GAS is expected to be mediated by streptokinase (SK). (Bottom) Progression curve of plasminogen activation in the presence or absence of recombinant SK.

FIG. 13: hPLG is recruited to the CDI-damaged gut, exacerbates tissue damage and disease and aids in spore dissemination in the infected host. (A) Representative PAS/Alcian blue stained cecal sections from hPLG transgenic mice or hPLG infused WT mice that were either uninfected or infected with C. difficile M7404 WT or an M7404 toxin mutant (A-B-). Square brackets ([) indicate crypt hyperplasia, arrow heads (▴) represent epithelial damage, and asterisks (*) represent edema and inflammation. Scale bar represents 200 μm. (B) Western blots of standardized amounts of hPLG from the cecum of hPLG transgenic C. difficile uninfected versus infected mice. Purified hPLG is included as a positive control (lane 1). hPLG extracted from the cecum of uninfected hPLG (lane 2) and C. difficile infected hPLG mice (lane 3) and from the cecum of hPLG infused uninfected (lane 4), M7404 toxin mutant (A-B-) (lane 5) or M7404 infected C57BL/6 mice (lane 6) is shown. Units (100 and 75) refer to the molecular weight of protein standards, in kDa. (C) Fecal consistency, (D) Cage appearance and (E) Physiological appearance. (F) Survival time for hPLG transgenic (n=20) and C57BL/6 (n=13) C. difficile M7404 infected mice, uninfected controls for hPLG (n=7) and C57BL/6 (n=11) mice. (G, H) Fecal toxin levels from C. difficile infected hPLG transgenic mice or C57BL/6 mice, assessed using HT29 cells (TcdA) (G) and Vero cells (TcdB). (H) No statistically significant differences in toxin levels were observed. (I) C. difficile spore numbers in the kidney, spleen and thymus of C. difficile infected hPLG transgenic mice or C57BL/6 mice. (J) Inflammation of hPLG transgenic infected and uninfected mice. (K) Survival times for mPLG KO (n=18) and C57BL/6L (n=18) C. difficile M7404 infected mice, uninfected controls for mPLG KO (n=5) and C57BL/6 (n=6) mice. Statistical analysis was determined using a one way Anova with Tukey's multiple comparisons (** P<0.005; ***P<0.0005; ****P<0.0001) (C, D, E, J) or using a Mann Whitney test; * P<0.05; **P<0.001; ***P<0.0001) (F, G, H, I, K).

FIG. 14: hPLG alters the host inflammatory response and tissue integrity during CM. (A) Cytokine levels using a 22 plex cytokine array and TIMP-1 ELISA on tissue lysates from the cecum of either PBS infused (open bars) or hPLG infused (closed bars) C. difficile M7404 infected mice are shown. The fold ratio represents the cytokine levels present in the gut tissues of infected mice normalised to levels obtained from each respective uninfected mouse group. Only the cytokine levels that differed between the hPLG and PBS infused and infected mice are shown. (B) The abundance (Login) of the same cytokines that differed between the infected hPLG infused (orange squares) and infected PBS infused (black circles) mice are shown for the cecum of each uninfected mouse group. Note that to confirm that altered cytokine expression did not result from the presence of hPLG alone, the cytokine levels in uninfected mice were examined. The cytokine levels in hPLG infused but uninfected mice were slightly, but not significantly, lower than those in the PBS infused uninfected mice, however, cytokine levels in both uninfected groups were low when compared to those in infected mice. Statistical analysis was determined using a Mann Whitney test (* P<0.05; **P<0.001; ***P<0.0001).

FIG. 15: Human, equine and porcine but not murine PLG binds to C. difficile spores. (A) Western blot of C. difficile M7404 vegetative cells using an anti-human PLG Ab. Samples representing vegetative cells incubated in the absence (V) or presence of hPLG (VP) are indicated. (B) Representative Biacore sensor curves for the interaction between human, horse and porcine PLG and M7404 spores (black squares, purple circles, orange triangles, respectively) and lack of interaction between mouse PLG and spores (blue diamonds). Data were fit to a one site specific binding model. (C) Binding of hPLG to C. difficile spores isolated from strains from diverse locations and origins (M7404 Canadian human epidemic isolate (E1); R20291 UK human epidemic isolate (E2); JGS6133 US animal isolate (A-US); A135 Australian animal isolate (A-AU); DLL3109 Australian human epidemic isolate (E-AU); VP110463 US human reference isolate (R) and CD37 US non-toxigenic isolate (NT)) and detected using an anti-human PLG Ab. For (A) and (C), samples representing spores incubated in the absence (S) or presence of hPLG (SP) are indicated. Purified hPLG (positive control) protein (P) and supernatants from spores washed 5 times with binding buffer to remove any unbound PLG (W) are also included as controls. Units (100 and 75) refer to the molecular weight of protein standards, in kDa. (D-F) Stimulated emission depletion (STED) microscopy of in vitro derived M7404 spores stained using an anti-spore antibody (green) bound to hPLG (red). (G-I) STED microscopy of human derived faecal C. difficile spores stained using an anti-spore antibody (green) bound to hPLG (red). (J-L) STED microscopy of mouse derived faecal M7404 spores. Anti-spore antibody staining is shown in green; hPLG staining is shown in red. Merged STED images for in vitro derived spores, human faecal derived spores and mouse faecal derived spores are shown in F, I and L, respectively.

FIG. 16: Plasmin binding to C. difficile spores remodels the spore surface and increases germination efficiency. Transverse TEM sections of (A) untreated or (B) plasmin bound M7404 spores. The boxed regions from (A) and (B) are shown enlarged in (C) and (D), respectively. (E) The average length (nm) of the exosporium was measured from untreated and plasmin-bound M7404 spores (n=40 spores/group, with 5 measurements per spore). Statistical analysis was determined using a Mann-Whitney U test (P<0.0001). (F) Germination rates of untreated (orange squares) or plasmin bound (blue triangle) M7404 spores in the presence of 50 μM sodium taurocholate. Untreated spores incubated in the absence of sodium taurocholate (black squares) were included as a negative germination control. Data represents the ratio of the OD₆₀₀ at each time point (OD₆₀₀(t)) over the OD₆₀₀ at time 0 (OD₆₀₀(t₀) and is measured over time (mins). Statistical analysis was determined using a 2way Anova with Tukey's multiple comparisons test (P<0.01 for untreated spores versus plasmin-bound spores in the presence of germinant).

FIG. 17: Reduction in disease severity, delay in onset and increase in survival of mice infected with C. difficile when administered anti-plasminogen antibody. (A) Cage appearance (B) Faecal consistency (C) Physiological appearance and (D) Survival curve of hPLG infused and C. difficile M7404 infected mice either injected IP with hepes alone, the G05 antibody or the naïve chicken antibody. Statistical analysis was determined using a Mann Whitney test; * P<0.05.0.

FIG. 18: Cross-reactivity of G05 and G11 antibodies. The G05 antibody does not significantly inhibit related serine proteases, including plasmin. The G11 antibody does not significantly inhibit related serine proteases, with the exception of plasmin.

FIG. 19: Administration of G05 antibody reduces bleeding. Bleeding volume (as measured in OD₅₅₀ nm) from wild type C56BL/6 (WT) mice and mice harbouring a homozygous loss-of-function mutation in the gene encoding plasminogen (Plg^{-/-}) subjected to tail bleed experiments. All animals received 200 μg of human plasminogen and 50 μg of the antibody, either gAb (negative control) or G05. The bleeding volume for mice that received G05 antibody was significantly reduced compared to mice that received naïve antibody.

FIG. 20: G11 inhibits whole blood clot lysis in the presence of red blood cells and platelets. The time required to achieve 50% whole blood clot lysis (IC₅₀ by G11, α2AP and Aprotinin are shown. G11 is approximately 2-fold more efficacious that α2AP and approximately 2.5-fold more efficacious that Aprotinin in inhibiting whole blood clot lysis.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present inventors have developed antigen binding proteins, for example antibodies, that bind to and inhibit or reduce the activation of plasminogen.

By virtue of their ability to bind to and inhibit or reduce activation of plasminogen, and as further explained herein, the antigen binding proteins of the invention are useful for treating, preventing or delaying the progression of conditions or diseases mediated by plasminogen activation to plasmin. For example, the antigen binding proteins of the invention are useful for inhibiting fibrinolysis and for promoting haemostasis following trauma, or haemorrhage following surgery, child-birth, or any other circumstance where inhibition of plasminogen activation is required.

The antigen binding proteins of the invention also have utility for treating or preventing bacterial infections where the bacterial pathogen recruits the plasmin system to promote invasion of host tissues. In particular, the antigen binding proteins of the invention are useful for treating or preventing infections caused by Streptococcus sp., Staphylococcus sp, Yersinia pestis, Helicobacter pylori, E. coli, Salmonella sp., and P. gingivalis.

The plasmin system can also be employed by invading tumours to promote angiogenesis and metastasis. Accordingly, the antigen binding proteins of the invention also have the capacity to inhibit or reduce one or more aspects of the inflammatory, tumour growth and metastatic activity.

General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65” will be understood in an inclusive manner, i.e., the region comprises a sequence of amino acids as numbered 56, 57, 58, 59, 60, 61, 62, 63, 64 and 65 in a specified sequence.

### Selected Definitions

Plasminogen is the inactive precursor form of plasmin, the principal fibrinolytic enzyme in mammals. Plasmin also plays an important role in cell migration, tissue remodeling, and bacterial invasion. Plasmin is a serine protease that preferentially cleaves Lys-Xaa and Arg--Xaa bonds with higher selectivity than trypsin. Plasminogen activators such as tissue plasminogen activator (tPA) or urokinase cleave human plasminogen molecule at the Arg₅₆₁-Val₅₆₂ bond to produce active plasmin. The two resulting chains of plasmin are held together by two interchain disulphide bridges. The light chain (25 kDa) carries the catalytic center (which comprises the catalytic triad) and shares sequence similarity with trypsin and other serine proteases. The heavy chain (60 kDa) consists of five highly similar triple-loop structures called kringles. Some of the kringles contain lysine binding sites that mediates the plasminogen/plasmin interaction with fibrin. Plasmin belongs to peptidase family Si.

Plasminogen (or Plg) is a seven-domain glycoprotein comprising a Pap or pan-apple domain, 5 kringle domains (KR 1 to KR5) and a serine protease (SP) domain. Plg circulates in a closed and activation-resistant conformation in plasma. Upon localization to a target site, plasminogen binds to the surface lysine/arginine residues on the targets (which include fibrin clots and cell surface receptors). Binding occurs via lysine-binding sites (LBSs) on the kringle domains, an event that triggers a structural re-arrangement of plasminogen to an open conformation. Upon converting from closed to open conformation, the activation loop between KR-5 and SP domains becomes exposed, and is cleaved by plasminogen activators (such as tissue plasminogen activator or urokinase plasminogen activator) to form the enzymatically active form, plasmin (Plm). The plasminogen activation system is tightly regulated by host serine protease inhibitors: plasminogen activation inhibitors 1 and 2 (PAI-1 and PAI-2). Active plasmin released from targets is typically removed from circulation by specific inhibitors alpha-2-antiplasmin or the housekeeping enzyme alpha-2-macroglobulin.

The term “plasminogen” as provided herein includes any of the variants of plasminogen, including Glu-plasminogen (Glu-Plg), Lys-plasminogen (Lys-Pig), and mini-, midi- and micro-plasminogens. Lys-plasminogen is an N-truncated form of Glu-Plg that is formed from the cleavage of Glu-plasminogen by plasmin. Lys-plasminogen exhibits higher affinity for fibrin compared to Glu-Plg and is better activated by uPA and tPA. Midi-plasminogen comprises kringle domains 4 and 5 and the light chain (serine protease domain) of plasminogen. It is formed by cleavage of kringle domains 1 to 3 from Glu-plasminogen. Mini-plasminogen (also known as 442Val-Plg or neoplasminogen) results from the action of elastase on Glu-plasminogen at residue 442 (located within Kringle domain 4). Thus mini-plasminogen comprises part of kringle domain 4, kringle domain 5 and the serine protease domain of plasminogen. Micro-plasminogen consists of the proenzyme domain of plasminogen with a stretch of connecting peptide and a few residues of kringle 5 attached at its N-terminal end. It is produced by the action of plasmin on plasminogen. Thus, micro-plasminogen (or micro-Pig) comprises the light chain of plasminogen (serine protease domain) and no kringle domains. (See, for example, Shi et al. (1980) J Biol. Chem. 263:17071-5). Like plasminogen, microplasminogen is activated by tPA and urokinase to form a proteolytically active molecule. Human microplasmin has a molecular weight of approximately 29 kDa and has a lower affinity for fibrin when compared with plasmin.

For the purposes of nomenclature only and not a limitation, an exemplary amino acid sequence of human plasminogen (“glu-PLG) is set forth in SEQ ID NO: 65. The sequence comprising the hPlg activation loop is set forth in SEQ ID NO: 66.

As used herein, reference to plasminogen is to a molecule that has at least one biochemical or biophysical activity of plasminogen. The biochemical or biophysical activities, and structure of plasminogen can be distinguished from those of plasmin.

The phrase “inhibits plasminogen activation” or “reduces plasminogen activation” is understood to mean that the antigen binding protein of the present invention inhibits or reduces the conversion of plasminogen to plasmin. Further, the activity is measured using a suitable in vitro, cellular or in vivo assay and the activity is blocked or reduced by at least 1%, 5%, 10%, 25%, 50%, 60%, 70%, 80% or 90% or more, compared to plasminogen activation in the same assay under the same conditions but without the antigen binding protein. Preferably, the plasminogen activation is mediated or induced by any one or more plasminogen activators. A plasminogen activator is any enzyme that can cleave the Arg₅₆₁-Val₅₆₂ bond (numbering as per human plasminogen). Exemplary plasminogen-cleaving serine proteases, therefore plasminogen activators, include the coagulation proteins factor IX, factor X, and prothrombin (factor II), protein C, chymotrypsin and trypsin, various leukocyte elastases, the streptokinase (SK), urokinase (uPA) and tissue plasminogen activator (tPA), and plasmin. Preferably, the plasminogen activator is selected from the group consisting of streptokinase (SK), urokinase (uPA) and tissue plasminogen activator (tPA). Preferably the inhibition or reduction in activation of plasminogen is in the presence of one or more cofactors. Preferably, the cofactors are selected from the group consisting of ε-aminocaproic acid (EACA), fibrinogen (Fg) and fibrin (Fn)

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions. The protein may include one or more non-natural amino acids.

The term “polypeptide” or “polypeptide chain” will be understood from the foregoing paragraph to mean a series of contiguous amino acids linked by peptide bonds.

As used herein, the term “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens (e.g., plasminogen) by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies).

An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework regions” (FRs) are those variable region residues other than the CDR residues. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

As used herein, the term “Fv” shall be taken to mean any protein, whether comprised of multiple polypeptides or a single polypeptide, in which a VL and a VH associate and form a complex having an antigen binding domain, i.e., capable of specifically binding to an antigen. The VH and the VL which form the antigen binding domain can be in a single polypeptide chain or in different polypeptide chains. Furthermore, an Fv of the invention (as well as any protein of the invention) may have multiple antigen binding domains which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labeled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells. For example, an antigen binding protein binds to plasminogen (e.g., human plasminogen) with materially greater affinity (e.g., 1.5 fold or 2 fold or 5 fold or 10 fold or 20 fold or 40 fold or 60 fold or 80 fold to 100 fold or 150 fold or 200 fold) than it does to other related molecules, such as other serine proteases. In an example of the present invention, an antigen binding protein that “specifically binds” to plasminogen (preferably human) with an affinity at least 1.5 fold or 2 fold or greater (e.g., 5 fold or 10 fold or 20 fold r 50 fold or 100 fold or 200 fold) than it does to plasmin. Generally, but not necessarily, reference to binding means specific binding, and each term shall be understood to provide explicit support for the other term.

As used herein, the term “does not detectably bind” shall be understood to mean that an antigen binding protein, e.g. an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding protein is immobilized and contacted with an antigen.

As used herein, the term “does not significantly bind” shall be understood to mean that the level of binding of an antigen binding protein of the invention to a polypeptide is not statistically significantly higher than background, e.g., the level of binding signal detected in the absence of the antigen binding protein and/or in the presence of a negative control protein (e.g., an isotype control antibody) and/or the level of binding detected in the presence of a negative control polypeptide. The level of binding is detected using biosensor analysis (e.g. Biacore) in which the antigen binding protein is immobilized and contacted with an antigen.

As used herein, the term “epitope” (syn. “antigenic determinant”) shall be understood to mean a region of plasminogen to which an antigen binding protein comprising an antigen binding domain of an antibody binds. Unless otherwise defined, this term is not necessarily limited to the specific residues or structure to which the antigen binding protein makes contact. For example, this term includes the region spanning amino acids contacted by the antigen binding protein and 5-10 (or more) or 2-5 or 1-3 amino acids outside of this region. In some examples, the epitope comprises a series of discontinuous amino acids that are positioned close to one another when antigen binding protein is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

As used herein, the term “condition” refers to a disruption of or interference with normal function, and is not to be limited to any specific condition, and will include diseases or disorders.

As used herein, the terms “preventing”, “prevent” or “prevention” include administering an antigen binding protein of the invention to thereby stop or hinder the development of at least one symptom of a condition. This term also encompasses treatment of a subject in remission to prevent or hinder relapse.

As used herein, the terms “treating”, “treat” or “treatment” include administering an antigen binding protein described herein to thereby reduce or eliminate at least one symptom of a specified disease or condition.

As used herein, the term “subject” shall be taken to mean any animal including humans, for example a mammal. Exemplary subjects include but are not limited to humans and non-human primates. For example, the subject is a human.

Antibodies

In one example, an antigen binding protein or plasminogen-binding protein as described herein according to any example is an antibody.

Methods for generating antibodies are known in the art and/or described in Harlow and Lane (editors) Antibodies: A Laboratory Manual, Cold Spring Harbor Laboratory, (1988). Generally, in such methods plasminogen (e.g., human plasminogen) or a region thereof (e.g., an extracellular region) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human antibodies, for example, which do not express murine antibodies, can also be used to generate an antibody of the present invention (e.g., as described in WO2002/066630).

Following immunization, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemistry and/or immunoassay (e.g. radioimmunoassay, enzyme immunoassay, cytotoxicity assay, plaque assay, dot immunoassay, and the like).

Alternatively, ABL-MYC technology (NeoClone, Madison Wis. 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody of the present invention may be a synthetic antibody. For example, the antibody is a chimeric antibody, a humanized antibody, a human antibody synhumanized antibody, primatized antibody or a de-immunized antibody.

Antibody Binding Domain Containing Proteins

Single-Domain Antibodies

In some examples, a protein of the invention is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, Mass.; see, e.g., U.S. Pat. No. 6,248,516).

Diabodies, Triabodies, Tetrabodies

In some examples, a protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding domain, i.e., to form a Fv molecule capable of specifically binding to one or more antigens. The VL and VH can be the same in each polypeptide chain or the VL and VH can be different in each polypeptide chain so as to form a bispecific diabody (i.e., comprising two Fvs having different specificity).

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

The present invention also contemplates a disulfide stabilized Fv (or diFv or dsFv), in which a single cysteine residue is introduced into a FR of V_{H} and a FR of VL and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “V_{HH} domains” in camelid antibodies and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

Other Antibodies and Proteins Comprising Antigen Binding Domains Thereof

The present invention also contemplates other antibodies and proteins comprising antigen-binding domains thereof, such as:

(i) “key and hole” bispecific proteins as described in U.S. Pat. No. 5,731,168;

(ii) heteroconjugate proteins, e.g., as described in U.S. Pat. No. 4,676,980;

(iii) heteroconjugate proteins produced using a chemical cross-linker, e.g., as described in U.S. Pat. No. 4,676,980; and

(iv) Fab₃ (e.g., as described in EP19930302894).

Mutations to Proteins

The present invention also provides an antigen binding protein or a nucleic acid encoding same having at least 80% identity to a sequence disclosed herein. In one example, an antigen binding protein or nucleic acid of the invention comprises sequence at least about 85% or 90% or 95% or 97% or 98% or 99% identical to a sequence disclosed herein.

Alternatively, or additionally, the antigen binding protein comprises a CDR (e.g., three CDRs) at least about 80% or 85% or 90% or 95% or 97% or 98% or 99% identical to CDR(s) of a V_{H} or V_{L} as described herein according to any example.

In another example, a nucleic acid of the invention comprises a sequence at least about 80% or 85% or 90% or 95% or 97% or 98% or 99% identical to a sequence encoding an antigen binding protein having a function as described herein according to any example. The present invention also encompasses nucleic acids encoding an antigen binding protein of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridizes under stringent hybridization conditions to a nucleic acid encoding an antigen binding site described herein. A “moderate stringency” is defined herein as being a hybridization and/or washing carried out in 2×SSC buffer, 0.1% (w/v) SDS at a temperature in the range 45° C. to 65° C., or equivalent conditions. A “high stringency” is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

The present invention also contemplates mutant forms of an antigen binding protein of the invention comprising one or more conservative amino acid substitutions compared to a sequence set forth herein. In some examples, the antigen binding protein comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding protein comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding protein of the invention. In another example, the mutation(s) occur within a CDR of an antigen binding protein of the invention.

Exemplary methods for producing mutant forms of an antigen binding protein include:
mutagenesis of DNA (Thie et al., Methods Mol. Biol. 525: 309-322, 2009) or RNA (Kopsidas et al., Immunol. Lett. 107:163-168, 2006; Kopsidas et al. BMC Biotechnology, 7: 18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370: 389-91, 1994; and site directed mutagenesis, e.g., as described in Dieffenbach (ed) and Dveksler (ed) (In: PCR Primer: A Laboratory Manual, Cold Spring Harbor Laboratories, N Y, 1995).

Exemplary methods for determining biological activity of the mutant antigen binding proteins of the invention will be apparent to the skilled artisan and/or described herein, e.g., antigen binding. For example, methods for determining antigen binding, competitive inhibition of binding, affinity, association, dissociation and therapeutic efficacy are described herein.

Constant Regions

The present invention encompasses antigen binding proteins and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington D.C. United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one C_{H}2 domain from an IgG4 antibody and at least one C_{H}3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

Additional Modifications

The present invention also contemplates additional modifications to an antibody or antigen binding protein comprising an Fc region or constant region.

For example, the antibody comprises one or more amino acid substitutions that increase the half-life of the protein. For example, the antibody comprises a Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

Protein Production

In one example, an antigen binding protein described herein according to any example is produced by culturing a hybridoma under conditions sufficient to produce the protein, e.g., as described herein and/or as is known in the art.

Recombinant Expression

In another example, an antigen binding protein described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-lnterscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. Nos. 4,816,567 or 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (Ula and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

Isolation of Proteins

Methods for isolating a protein are known in the art and/or described herein.

Where an antigen binding protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Assaying Activity of an Antigen Binding Protein

Binding to Plasminogen and Mutants Thereof

It will be apparent to the skilled artisan from the disclosure herein that antigen binding protein of the present invention bind to plasminogen/plasmin. Methods for assessing binding to a protein are known in the art, e.g., as described in Scopes (In: Protein purification: principles and practice, Third Edition, Springer Verlag, 1994). Such a method generally involves immobilizing the antigen binding protein and contacting it with labeled antigen (plasminogen). Following washing to remove non-specific bound protein, the amount of label and, as a consequence, bound antigen is detected. Of course, the antigen binding protein can be labeled and the antigen immobilized. Panning-type assays can also be used. Alternatively, or additionally, surface plasmon resonance assays can be used.

Optionally, the dissociation constant (Kd), association constant (Ka) and/or affinity constant (K_{D}) of an immobilized antigen binding protein for plasminogen or an epitope thereof is determined. The “Kd” or “Ka” or “K_{D}” for an plasminogen-binding protein is in one example measured by a radiolabeled or fluorescently-labeled plasminogen ligand binding assay. In the case of a “Kd”, this assay equilibrates the antigen binding protein with a minimal concentration of labeled plasminogen or epitope thereof in the presence of a titration series of unlabeled plasminogen. Following washing to remove unbound plasminogen or epitope thereof, the amount of label is determined, which is indicative of the Kd of the protein.

According to another example the Kd, Ka or K_{D} is measured by using surface plasmon resonance assays, e.g., using BIAcore surface plasmon resonance (BIAcore, Inc., Piscataway, N.J.) with immobilized plasminogen or plasmin or a region thereof or immobilized antigen binding protein.

Determining Inhibitory Activity

The antigen binding proteins of the invention are preferably capable of inhibiting plasminogen activation and/or plasmin activity.

Various assays are known in the art for assessing the ability of a protein to inhibit or reduce activation of plasminogen to plasmin.

In one example, the antigen binding protein inhibits binding of any activator of plasminogen (plasminogen activator) from binding to plasminogen. Preferably, the antigen binding protein of the invention binds to plasminogen and prevents binding and/or cleavage of plasminogen in the activation loop, preferably wherein cleavage at Arg₅₆₁-Val₅₆₂ is prevented, reduced or inhibited.

Preferably, the antigen binding protein of the invention inhibits or prevents activation of plasminogen by any known plasminogen activator, including but not limited to: streptokinase (SK), tissue-plasminogen activator (tPA) or urokinase-plasminogen activator (uPA). The plasminogen activator whose activity is inhibited through the binding of the antigen binding protein of the invention to plasminogen, may be a plasminogen activator produced by a host organism (e.g., in a human) or may be secreted or produced by a bacterial pathogen to activate plasminogen in the host. Thus, the activation of plasminogen by either host or pathogen plasminogen activator may be inhibited or prevented by an antigen binding protein of the invention.

The antigen binding protein of the invention may also inhibit the binding of plasminogen to a pathogen, including inhibiting or preventing the binding of plasminogen to a bacterium. The inhibition may be in relation to the binding of plasminogen to a bacterial spore or to the vegetative form of the bacterium. The inhibition of the binding of plasminogen to the pathogen preferably inhibits activation of plasminogen to plasmin.

Exemplary methods for determining the inhibition of activation of plasminogen, inhibition of binding of plasminogen activators to plasminogen and inhibition of plasminogen binding to a pathogen are described herein, for example at Examples 2, 6 and 7.

Determining Inhibitory Activity

The antigen binding proteins of the invention may also be capable of inhibiting plasmin activity, including inhibiting plasmin-mediated clot lysis at comparable or significantly greater levels than physiological inhibitors of plasmin activity or pharmacological inhibitors of plasmin activity.

Various assays are known in the art for assessing the ability of a protein to inhibit or reduce plasmin activity.

In one example, the antigen binding protein inhibits proteolysis of any substrate by plasmin. Preferably, the antigen binding protein of the invention binds to plasmin and prevents binding and/or cleavage of a plasmin substrate by the serine protease domain. Preferably, the antigen binding protein of the invention binds to or sterically shields the catalytic triad of plasmin, such that the catalytic triad is unable to cleave a plasmin substrate, wherein the catalytic triad comprises the residues His₆₀₃, Asp₆₄₆ and Ala/Ser₇₄₁ of SEQ ID NO: 65 (which is equivalent to His₅₇, Asp₁₀₂ and Ser₁₉₅ using chymotrypsin numbering). Thus, in preferred embodiments, the antigen binding protein of the invention is an anti-catalytic antigen binding protein.

Preferably, the antigen binding protein of the invention inhibits or prevents cleavage of any known substrate of plasmin including but not limited to: fibrin, fibrinogen, Factors V, VIII and X, protease-activated receptor I, fibronectin, thrombospondin, laminin, von Willebrand factor, vitronectin, pro-brain-derived neurotrophic factor, complement C3 and C5, tenascin, osteocalin, CUB domain-containing protein 1 and other proteases such as collagenase.

Exemplary methods for determining the inhibition of plasmin activity are described herein, for example at Examples 2 and 10.

The antigen binding proteins of the invention also have utility in applications requiring detection of plasmin and/or plasminogen in a biological sample. For example, the antigen binding proteins of the invention may be useful for diagnostic applications including where the proteins are used in histology and ELISA and similar applications whereby binding of the antigen binding proteins to a target protein can provide useful diagnostic information.

Conditions to be Treated

The antigen binding proteins of the invention are useful in the treatment of conditions requiring inhibition of conversion of plasminogen to plasmin (i.e., inhibition of plasminogen activation) and/or inhibition of plasmin activity.

Active plasmin has exceptionally broad specificity for target substrates. Accordingly, plasmin targets include fibrin, fibrinogen, complement component 3, complement component 5, vitronectin, osteocalcin, factors V, VIII and X, protease-activated receptor, injury-induced aggregated proteins and some collagenases. Plasmin can also target the key plasminogen activators tPa and uPa to create a positive feedback loop.

Plasmin is a highly efficient enzyme, such that unchecked, plasmin can rapidly exhaust the entire reserve of circulating fibrinogen, resulting in a generalised haemorrhagic state within minutes. As such, under normal physiological conditions, active plasmin is only present on the surface of target sites such as fibrin clots, or cell surfaces.

In severe trauma patients, coagulopathy is frequently observed in the acute phase of trauma. Trauma-induced coagulopathy is coagulopathy caused by the trauma itself. The pathophysiology of trauma-induced coagulopathy consists of coagulation activation, hyperfibrino(geno)lysis, and consumption coagulopathy. These pathophysiological mechanisms are the characteristics to DIC with the fibrinolytic phenotype.

Fibrinolytic dysregulation is an important mechanism in traumatic coagulopathy. It is an incompletely understood process that consists of a spectrum ranging from excessive breakdown (hyperfibrinolysis) and the shutdown of fibrinolysis. Both hyperfibrinolysis and shutdown are associated with excess mortality and post-traumatic organ failure. The pathophysiology appears to relate to endothelial injury and hypoperfusion, with several molecular markers identified in playing a role. This condition is mediated, in part, by excessive upregulation of profibrinolytic tPA in the absence of concomitant increases in antifibrinolytic PAI-1, resulting in excessive activation of plasminogen to plasmin. Accordingly, in certain cases of trauma, there is a need to inhibit excessive plasminogen activation, which can be accomplished using the antigen binding proteins of the invention.

Still further, some conditions characterised by excessive plasminogen activation include conditions characterised by raised levels of a plasma plasminogen activator physically and immunologically related to that in human tissues and blood vessel endothelium. Thrombotic or haemorrhagic disorders due to abnormal fibrinolysis may also arise from congenital or acquired abnormalities of the fibrinolytic system.

The antigen binding proteins of the invention have utility in minimising or reducing haemorrhage, or bleeding, following surgery, injury or in individuals with coagulation factor deficiency. The use of the antigen binding proteins in this context inhibit plasmin-mediated fibrinolysis or clot dissolution, thereby reducing blood loss and reducing or minimising the requirement for blood transfusion. Blood transfusion is associated with a high risk of mismatch, allergic reactions, multi-organ dysfunction and infection, resulting in an increase in morbidity and mortality.

The antigen binding proteins of the invention may also be used to prevent bleeding in other conditions such as haemophilia, menorrhagia, von Willebrand syndrome and thrombolytic-induced bleeding.

The antigen binding proteins of the invention have utility in the inhibition of fibrinolysis in a number of clinical situations including to reduce bleeding in patients who have undergone cardiac surgery, orthopaedic surgery, neurosurgery, liver transplantation, vascular surgery, thoracic surgery, gynecological surgery, or who have end-stage renal disease, peripartum bleeding, gastrointestinal bleeding, trauma, traumatic brain injury, intracerebral bleeding and subarachnoid haemorrhage. In other words, the antigen binding proteins of the invention have utility in inhibiting plasmin in individuals that are in a hyperfibrinolytic state.

Accordingly, the antigen binding proteins of the invention have utility in inhibiting fibrinolysis in a broad range of scenarios where inhibition of plasminogen activation and/or inhibition of plasmin activity is required.

Other conditions requiring inhibition of plasminogen activation (and/or plasmin activity) include conditions characterised by excessive bleeding (haemorrhage) including after child-birth, and following surgery (including transplant, thoracic, cardiac and orthopaedic surgery).

The antigen binding proteins of the present invention are useful in the treatment or prevention of any condition associated, or caused by, the presence of or increased levels of bacterial pathogen.

Invasive bacterial pathogens intervene at various stages and by various mechanisms with the mammalian plasminogen/plasmin system. A vast number of pathogens express plasmin(ogen) receptors that immobilize plasmin(ogen) on the bacterial surface, an event that enhances activation of plasminogen by mammalian plasminogen activators. Bacteria also influence secretion of plasminogen activators and their inhibitors from mammalian cells. For example, the prokaryotic plasminogen activators streptokinase and staphylokinase form a complex with plasmin(ogen) and thus enhance plasminogen activation. The Pla surface protease of Yersinia pestis resembles mammalian activators in function and converts plasminogen to plasmin by limited proteolysis. In essence, plasminogen receptors and activators turn bacteria into proteolytic organisms using a host-derived system.

Some exemplary bacterial infections that can be treated/prevented with the antigen binding proteins of the invention are described further below. However, it will be understood that the use of the antigen-binding proteins of the invention is not limited to those bacterial infections described. More specifically, it will be understood that the antigen binding proteins of the invention find general application in treating/reducing the invasion of bacterial pathogens that secrete plasminogen activators for the purposes of invading host tissues

In Gram-negative bacteria, the filamentous surface appendages fimbriae and flagella form a major group of plasminogen receptors. In Gram-positive bacteria, surface-bound enzyme molecules as well as M-protein-related structures have been identified as plasminogen receptors, the former receptor type also occurs on mammalian cells. Consequently, plasmin generated on or activated by Haemophilus influenzae, Salmonella typhimurium, Streptococcus pneumoniae, Y. pestis, and Borrelia burgdorferi has been shown to degrade mammalian extracellular matrices. In a few instances plasminogen activation has been shown to enhance bacterial metastasis in vitro through reconstituted basement membrane or epithelial cell monolayers.

Streptococcus pyogenes, or Group A Streptococcus (GAS), is a facultative, Gram-positive coccus which grows in chains and causes numerous infections in humans including pharyngitis, tonsillitis, scarlet fever, cellulitis, erysipelas, rheumatic fever, post-streptococcal glomerulonephritis, necrotizing fasciitis, myonecrosis and lymphangitis.

Streptokinase (SK), secreted by Streptococcus sp., in particular, can overcome immune defences and activate plasminogen in circulation to facilitate infection. Streptokinase has been reported to directly activate the closed conformation of plasminogen by wrapping around the serine protease domain of plasminogen, and once bound, the N-terminus of streptokinase inserts into the protease domain activation loop to produce active plasmin. The antigen binding proteins of the present invention have been demonstrated to inhibit the binding of streptokinase to plasminogen and thereby inhibit the activation of plasminogen by streptokinase. A similar, although slightly different mechanism of activation of plasminogen is understood to occur upon binding of staphylokinase, an important virulence factor secreted by Staphylococcus spp. Accordingly, the antigen binding proteins of the present invention have particular utility in treating, minimising the severity of, or delaying the progression of infections caused by gram positive bacteria such as Streptococcus sp, Staphylococcus sp. and Enterococcus sp.

The antigen binding proteins of the invention are also useful for treating infections caused by invasive gram-negative bacteria such Helicobacter pylori, Yersinia pestis, Salmonella sp. Escherichia spp, Campylobacter spp. and Shigella spp., and the spirochete Borellia (the causative agent of Lyme disease).

As used herein, any treatment of a bacterial infection, includes reduction in the severity of the symptoms or signs of an infection with a microbe or preventing the progression or worsening of the infection. It will be understood that such treatment, reduction or prevention includes reducing the number of bacteria or preventing the spread of the bacteria to other sites within the host (i.e., reducing the invasion of the pathogen).

In particular, the present inventors have shown that promotion of sporulation and virulence by Clostridium difficile is mediated by plasminogen activation. In particular, the inventors demonstrate that plasminogen specifically binds to C. difficile spores and active plasmin degrades their surface, facilitating rapid germination. More specifically, binding and activation of human plasminogen directly changes the structural and functional properties of C. difficile spores, leading to faster germination in the presence of a physiologically relevant host germinant. This may contribute to earlier disease onset and severe disease outcomes. The antigen-binding proteins of the present invention have been demonstrated to have particular utility in reducing the severity of infection with Clostridium difficile (also known as Clostroides difficile or CDI).

Plasminogen activation is reported to be associated with promotion of angiogenesis including in the proliferation/metastasis of cancer. Further, it has been demonstrated that inhibitors of plasminogen activation (e.g., PAI-1) are useful for inhibiting angiogenesis in the context of cancer.

Accordingly, the antibodies of the present invention also find use in treating, or delaying the progression of cancer, or metastasis of cancer in a subject. Exemplary cancers include cystic and solid tumors, bone and soft tissue tumors, including tumors in anal tissue, bile duct, bladder, blood cells, bowel, brain, breast, carcinoid, cervix, eye, esophagus, head and neck, kidney, larynx, leukemia, liver, lung, lymph nodes, lymphoma, melanoma, mesothelioma, myeloma, ovary, pancreas, penis, prostate, skin (e.g. squamous cell carcinoma), sarcomas, stomach, testes, thyroid, vagina, vulva. Soft tissue tumors include Benign schwannoma Monosomy, Desmoid tumor, lipo-blastoma, lipoma, uterine leiomyoma, clear cell sarcoma, dermatofibrosarcoma, Ewing sarcoma, extraskeletal myxoid chondrosarcoma, I iposarcoma myxoid, Alveolar rhabdomyosarcoma and synovial sarcoma. Specific bone tumors include nonossifying fibroma, unicameral bone cyst, enchon-droma, aneurismal bone cyst, osteoblastoma, chondroblastoma, chondromyxofibroma, ossifying fibroma and adamantinoma, Giant cell tumor, fibrous dysplasia, Ewing's sarcoma eosinophilic granuloma, osteosarcoma, chondroma, chondrosarcoma, malignant fibrous histiocytoma and metastatic carcinoma. Leukemias include acute lymphoblastic, acute myeloblastic, chronic lymphocytic and chronic myeloid.

Other examples include breast tumors, colorectal tumors, adenocarcinomas, mesothelioma, bladder tumors, prostate tumors, germ cell tumor, hepatoma/cholongio, carcinoma, neuroendocrine tumors, pituitary neoplasm, small 20 round cell tumor, squamous cell cancer, melanoma, atypical fibroxanthoma, seminomas, nonseminomas, stromal leydig cell tumors, Sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, vaginal tumors and Wilm's tumor.

As used herein, a “subject” refers to an animal, such as a mammalian or an avian species, including a human, an ape, a horse, a cow, a sheep, a goat, a dog, and a cat.

The subject may have a bacterial infection, may have been exposed to infectious bacteria may be at risk for developing a bacterial infection, or may be at greater risk than the general population for developing a bacterial infection. Examples of subjects at greater risk for developing a bacterial infection include patients undergoing treatment for bacterial infections whereby normal gut flora is inhibited by antibacterial therapy, patients with impaired immune function (e.g., immunoglobulin deficiency, splenic dysfunction, splenectomy, HIV infection, impaired leukocyte function, hemoglobinopathies), the elderly, people with certain malignancies (e.g., multiple myeloma, chronic lymphocytic leukemia, lymphoma), people at increased occupational risk (e.g., public services workers, such a fire, water, sanitary, police, medical, and laboratory workers, hospital workers), people in closed populations (e.g., prisons, military, nursing homes) and others that have immunological deficiencies that might enhance their susceptibility to bacterial infection.

A bacterial infection generally refers to:

(1) an elevated level of bacteria in a sample taken from the subject compared to an uninfected control sample;

(2) an increased proportion of one or more types of bacteria in a sample taken from the subject compared to the total level of bacteria in an uninfected control sample;

(3) an increased proportion of bacteria relative to one or more other bacteria species in a sample taken from the subject when compared to an uninfected control sample; or

(4) the presence of a bacteria in a sample compared to an uninfected control sample when that same bacteria is undetectable in the uninfected control.

A subject may be diagnosed as having a bacterial infection by any method described herein or known in the art. A biological sample such as a bodily fluid sample (e.g. blood) or tissue sample or scraping. Then the sample is prepared (various ways) and then cultured on different agar plates with defined media that will classify the microbe. Real time PCR is another method that may be used to identify bacteria in a sample.

An acute infection refers to an infection in a subject that requires rapid treatment, generally in the range of 15 to 60 minutes, otherwise the infection may progress to endanger the life of the subject. Such acute infections may occur in infants or immunocompromised subjects as described herein.

In one aspect, the terms “infection” and “bacterial infection” refer to an infection caused by Gram-positive bacteria, also referred to as a “Gram-positive infection”.

Gram-positive bacteria refer to bacteria that are stained blue or violet by gram staining, and include, for example, Clostroides difficile (Clostridium difficile), Streptococcus pyrogenes, Staphylococcus aureus, Lactobacillus spp, Bifidobacteria and Scardovia wiggsiae, Bacillus anthracis and the like. Gram-positive bacteria feature of having a thick peptidoglycan layer around a cell membrane and having no outer membrane on a periphery of the cell membrane. Gram-positive bacteria is not limited to Gram-positive cocci or Gram-positive bacilli.

The antigen-binding proteins of the present invention may be useful for reducing the severity or preventing toxic shock. Symptoms of toxic shock or toxic shock syndrome (TSS) vary depending on the underlying cause. TSS resulting from infection with the bacterium Staphylococcus aureus typically manifests in otherwise healthy subjects via signs and symptoms including high fever, accompanied by low blood pressure, malaise and confusion, which can rapidly progress to stupor, coma, and multiple organ failure. The characteristic rash, often seen early in the course of illness, resembles a sunburn, and can involve any region of the body including the lips, mouth, eyes, palms and soles. In patients who survive the initial phase of the infection, the rash desquamates, or peels off, after 10-14 days.

In contrast, TSS caused by the bacterium Streptococcus pyogenes, or TSLS, typically presents in people with pre-existing skin infections with the bacteria. These subjects often experience severe pain at the site of the skin infection, followed by rapid progression of symptoms as described above for TSS. In contrast to TSS caused by Staphylococcus, streptococcal TSS less often involves a sunburn-like rash.

For staphylococcal toxic shock syndrome, the diagnosis is based strictly upon CDC criteria defined in 2011, as follows:
1. Body temperature>38.9° C. (102.02° F.)
2. Systolic blood pressure<90 mmHg
3. Diffuse macular erythroderma
4. Desquamation (especially of the palms and soles) 1-2 weeks after onset
5. Involvement of three or more organ systems:
   Gastrointestinal (vomiting, diarrhoea)
   Muscular: severe myalgia or creatine phosphokinase level at least twice the upper limit of normal
   Mucous membrane hyperaemia (vaginal, oral, conjunctival)
   Kidney failure (serum creatinine>2 times normal)
   Liver inflammation (bilirubin, AST, or ALT>2 times normal)
   Low platelet count (platelet count<100,000/mm³)
   Central nervous system involvement (confusion without any focal neurological findings)
6. Negative results of:
   Blood, throat, and CSF cultures for other bacteria (besides S. aureus)
   Negative serology for Rickettsia infection, leptospirosis, and measles Cases are classified as confirmed or probable based on the following:
   Confirmed: All six of the criteria above are met (unless the patient dies before desquamation can occur); and
   Probable: Five of the six criteria above are met.

In certain embodiments, the terms “infection” and “bacterial infection” refer to an infection caused by Gram-negative bacteria. The infection may be by an Enterobacteriacieae. An infection caused by “Enterobacteriaceae” refers to any of the Gram-negative bacteria in this family of bacteria which includes, but is not limited to, species such as Salmonella spp., Escherichia coli, Yersinia pestis, Klebsiella spp., Shigella spp., Proteus spp., Enterobacter spp., Serratia spp., and Citrobacter spp. The infection may be caused by other gram-negative bacteria including Helicobacteraceae, such as Helicobacter pylori or Campylobacteraceae such as Camplyobacter jejuni.

Exemplary bacteria which can result in an infection and which the present invention finds particular application in the treatment, prevention or prophylaxis of are now described below. Also described in the context of the different types of bacteria are examples conditions associated with, or caused by, a bacterial infection comprising or consisting of that type of bacteria. These examples will be understood not to limit the uses of the antigen binding proteins of the invention, which have utility in a range of conditions associated with bacteria that utilise plasminogen activation to promote pathogenesis.

Escherichia coli (E. coli) is a Gram-negative bacterium that is part of the normal flora of the gastrointestinal tract. There are hundreds of strains of E. coli, most of which are harmless and live in the gastrointestinal tract of healthy humans and animals. Currently, there are four recognized classes of enterovirulent E. coli (the “EEC group”) that cause gastroenteritis in humans. Among these are the enteropathogenic (EPEC) strains and those whose virulence mechanism is related to the excretion of typical E. coli enterotoxins. Such strains of E. coli can cause various diseases including those associated with infection of the gastrointestinal tract and urinary tract, septicemia, pneumonia, and meningitis. Antibiotics are not effective against some strains and do not necessarily prevent recurrence of infection.

For example, E. coli strain O157:H7 is estimated to cause 10,000 to 20,000 cases of infection in the United States annually (Federal Centers for Disease Control and Prevention). Hemorrhagic colitis is the name of the acute disease caused by E. coli O157:H7. Preschool children and the elderly are at the greatest risk of serious complications.

Exemplary sequences for enterovirulent E. coli strains include GenBank Accession Numbers AB011549, X97542, AF074613, Y11275 and AJ007716.

Salmonella typhimurium, are Gram-negative bacteria which cause various conditions that range clinically from localized gastrointestinal infections, gastroenteritis (diarrhea, abdominal cramps, and fever) to enteric fevers (including typhoid fever) which are serious systemic illnesses. Salmonella infection also causes substantial losses of livestock.

Typical of Gram-negative bacilli, the cell wall of Salmonella spp. contains a complex lipopolysaccharide (LPS) structure that is liberated upon lysis of the cell and may function as an endotoxin, which contributes to the virulence of the organism.

Contaminated food is the major mode of transmission for non-typhoidal salmonella infection, due to the fact that Salmonella survive in meats and animal products that are not thoroughly cooked. The most common animal sources are chickens, turkeys, pigs, and cows; in addition to numerous other domestic and wild animals. The epidemiology of typhoid fever and other enteric fevers caused by Salmonella spp. is associated with water contaminated with human feces.

Pseudomonas spp. are motile, Gram-negative rods which are clinically important because they are resistant to most antibiotics, and are a major cause of hospital acquired (nosocomial) infections. Infection is most common in: immunocompromised subjects, burn victims, subjects on respirators, subjects with indwelling catheters, IV narcotic users and subject with chronic pulmonary disease (e.g., cystic fibrosis). Although infection is rare in healthy subjects, it can occur at many sites and lead to urinary tract infections, sepsis, pneumonia, pharyngitis, and numerous other problems, and treatment often fails with greater significant mortality.

Pseudomonas aeruginosa is a Gram-negative, aerobic, rod-shaped bacterium with unipolar motility. An opportunistic human pathogen, P. aeruginosa is also an opportunistic pathogen of plants. Like other Pseudomonads, P. aeruginosa secretes a variety of pigments. Definitive clinical identification of P. aeruginosa can include identifying the production of both pyocyanin and fluorescein as well as the organism's ability to grow at 42° C. P. aeruginosa is also capable of growth in diesel and jet fuel, for which it is known as a hydrocarbon utilizing microorganism (or “HUM bug”), causing microbial corrosion.

Vibrio cholerae is a Gram-negative rod which infects humans and causes cholera, a disease spread by poor sanitation, resulting in contaminated water supplies. Vibrio cholerae can colonize the human small intestine, where it produces a toxin that disrupts ion transport across the mucosa, causing diarrhea and water loss. Subjects infected with Vibrio cholerae require rehydration either intravenously or orally with a solution containing electrolytes. The illness is generally self-limiting; however, death can occur from dehydration and loss of essential electrolytes. Antibiotics such as tetracycline have been demonstrated to shorten the course of the illness, and oral vaccines are currently under development.

Neisseria gonorrhoea is a Gram-negative coccus, which is the causative agent of the common sexually transmitted disease, gonorrhea. Neisseria gonorrhoea can vary its surface antigens, preventing development of immunity to reinfection. Nearly 750,000 cases of gonorrhea are reported annually in the United States, with an estimated 750,000 additional unreported cases annually, mostly among teenagers and young adults. Ampicillin, amoxicillin, or some type of penicillin used to be recommended for the treatment of gonorrhea. However, the incidence of penicillin-resistant gonorrhea is increasing, and new antibiotics given by injection, e.g., ceftriaxone or spectinomycin, are now used to treat most gonococcal infections.

Staphylococcus aureus is a Gram-positive coccus which normally colonizes the human nose and is sometimes found on the skin. Staphylococcus can cause bloodstream infections, pneumonia, and surgical-site infections in the hospital setting (i.e., nosocomial infections). Staph. aureus can cause severe food poisoning, and many strains grow in food and produce exotoxins. Staphylococcus resistance to common antibiotics, e.g., vancomycin, has emerged in the United States and abroad as a major public health challenge both in community and hospital settings. Recently, a vancomycin-resistant Staph. aureus isolate has also been identified in Japan.

Mycobacterium tuberculosis is a Gram positive bacterium which is the causative agent of tuberculosis, a sometimes crippling and deadly disease. Tuberculosis is on the rise and globally and the leading cause of death from a single infectious disease (with a current death rate of three million people per year). It can affect several organs of the human body, including the brain, the kidneys and the bones, however, tuberculosis most commonly affects the lungs.

In the United States, approximately ten million individuals are infected with Mycobacterium tuberculosis, as indicated by positive skin tests, with approximately 26,000 new cases of active disease each year. The increase in tuberculosis (TB) cases has been associated with HIV/AIDS, homelessness, drug abuse and immigration of persons with active infections. Current treatment programs for drug-susceptible TB involve taking two or four drugs (e.g., isoniazid, rifampin, pyrazinamide, ethambutol or streptomycin), for a period of from six to nine months, because all of the TB germs cannot be destroyed by a single drug. In addition, the observation of drug-resistant and multiple drug resistant strains of Mycobacterium tuberculosis is on the rise.

Helicobacter pylori (H. pylori) is a micro-aerophilic, Gram-negative, slow-growing, flagellated organism with a spiral or S-shaped morphology which infects the lining of the stomach. H. pylori is a human gastric pathogen associated with chronic superficial gastritis, peptic ulcer disease, and chronic atrophic gastritis leading to gastric adenocarcinoma. H. pylori is one of the most common chronic bacterial infections in humans and is found in over 90% of patients with active gastritis. Current treatment includes triple drug therapy with bismuth, metronidazole, and either tetracycline or amoxicillin which eradicates H. pylori in most cases. Problems with triple therapy include patient compliance, side effects, and metronidazole resistance. Alternate regimens of dual therapy which show promise are amoxicillin plus metronidazole or omeprazole plus amoxicillin.

Streptococcus pneumoniae is a Gram-positive coccus and one of the most common causes of bacterial pneumonia as well as middle ear infections (otitis media) and meningitis. Each year in the United States, pneumococcal diseases account for approximately 50,000 cases of bacteremia; 3,000 cases of meningitis; 100,000-135,000 hospitalizations; and 7 million cases of otitis media. Pneumococcal infections cause an estimated 40,000 deaths annually in the United States. Children less than 2 years of age, adults over 65 years of age and persons of any age with underlying medical conditions, including, e.g., congestive heart disease, diabetes, emphysema, liver disease, sickle cell, HIV, and those living in special environments, e.g., nursing homes and long-term care facilities, at highest risk for infection.

Drug-resistant S. pneumoniae strains have become common in the United States, with many penicillin-resistant pneumococci also resistant to other antimicrobial drugs, such as erythromycin or trimethoprim-sulfamethoxazole.

Treponema pallidium is a spirochete which causes syphilis. T. pallidum is exclusively a pathogen which causes syphilis, yaws and non-venereal endemic syphilis or pinta. Treponema pallidum cannot be grown in vitro and does replicate in the absence of mammalian cells. The initial infection causes an ulcer at the site of infection; however, the bacteria move throughout the body, damaging many organs over time. In its late stages, untreated syphilis, although not contagious, can cause serious heart abnormalities, mental disorders, blindness, other neurologic problems, and death.

Haemophilus influenzae (H. influenza) is a family of Gram-negative bacteria; six types of which are known, with most H. influenza-related disease caused by type B, or “HIB”. Until a vaccine for HIB was developed, HIB was a common causes of otitis media, sinus infections, bronchitis, the most common cause of meningitis, and a frequent culprit in cases of pneumonia, septic arthritis (joint infections), cellulitis (infections of soft tissues), and pericarditis (infections of the membrane surrounding the heart). The H. influenza type B bacterium is widespread in humans and usually lives in the throat and nose without causing illness. Unvaccinated children under age 5 are at risk for HIB disease. Meningitis and other serious infections caused by H. influenza infection can lead to brain damage or death.

Shigella dysenteriae (Shigella dys.) is a Gram-negative rod which causes dysentary. In the colon, the bacteria enter mucosal cells and divide within mucosal cells, resulting in an extensive inflammatory response. Shigella infection can cause severe diarrhea which may lead to dehydration and can be dangerous for the very young, very old or chronically ill. Shigella dys. forms a potent toxin (shiga toxin), which is cytotoxic, enterotoxic, neurotoxic and acts as a inhibitor of protein synthesis. Resistance to antibiotics such as ampicillin and TMP-SMX has developed, however, treatment with newer, more expensive antibiotics such as ciprofloxacin, norfloxacin and enoxacin, remains effective.

Listeria is a genus of Gram-positive, motile bacteria found in human and animal feces. Listeria monocytogenes causes such diseases as listeriosis, meningoencephalitis and meningitis. This organism is one of the leading causes of death from food-borne pathogens especially in pregnant women, newborns, the elderly, and immunocompromised subjects. It is found in environments such as decaying vegetable matter, sewage, water, and soil, and it can survive extremes of both temperatures and salt concentration making it an extremely dangerous food-born pathogen, especially on food that is not reheated. The bacterium can spread from the site of infection in the intestines to the central nervous system and the fetal-placental unit. Meningitis, gastroenteritis, and septicemia can result from infection. In cattle and sheep, listeria infection causes encephalitis and spontaneous abortion.

Proteus mirabilis is an enteric, Gram-negative commensal organism, distantly related to E. coli. It normally colonizes the human urethra, but is an opportunistic pathogen that is the leading cause of urinary tract infections in catheterized subjects. P. mirabilis has two exceptional characteristics: 1) it has very rapid motility, which manifests itself as a swarming phenomenon on culture plates; and 2) it produce urease, which gives it the ability to degrade urea and survive in the genitourinary tract.

Yersinia pestis is the causative agent of plague (bubonic and pulmonary) a devastating disease which has killed millions worldwide. The organism can be transmitted from rats to humans through the bite of an infected flea or from human-to-human through the air during widespread infection. Yersinia pestis is an extremely pathogenic organism that requires very few numbers in order to cause disease, and is often lethal if left untreated. The organism is enteroinvasive, and can survive and propagate in macrophages prior to spreading systemically throughout the host.

Bacillus anthracis is also known as anthrax. Humans become infected when they come into contact with a contaminated animal. Anthrax is not transmitted due to person-to-person contact. The three forms of the disease reflect the sites of infection which include cutaneous (skin), pulmonary (lung), and intestinal. Pulmonary and intestinal infections are often fatal if left untreated. Spores are taken up by macrophages and become internalized into phagolysozomes (membranous compartment) whereupon germination initiates. Bacteria are released into the bloodstream once the infected macrophage lyses whereupon they rapidly multiply, spreading throughout the circulatory and lymphatic systems, a process that results in septic shock, respiratory distress and organ failure. The spores of this pathogen have been used as a terror weapon.

Burkholderia mallei is a Gram-negative aerobic bacterium that causes Glanders, an infectious disease that occurs primarily in horses, mules, and donkeys. It is rarely associated with human infection and is more commonly seen in domesticated animals. This organism is similar to B. pseudomallei and is differentiated by being nonmotile. The pathogen is host-adapted and is not found in the environment outside of its host. Glanders is often fatal if not treated with antibiotics, and transmission can occur through the air, or more commonly when in contact with infected animals. Rapid-onset pneumonia, bacteremia (spread of the organism through the blood), pustules, and death are common outcomes during infection. The virulence mechanisms are not well understood, although a type III secretion system similar to the one from Salmonella typhimurium is necessary. No vaccine exists for this potentially dangerous organism which is thought to have potential as a biological terror agent. The genome of this organism carries a large number of insertion sequences as compared to the related Bukholderia pseudomallei (below), and a large number of simple sequence repeats that may function in antigenic variation of cell surface proteins.

Burkholderia pseudomallei is a Gram-negative bacterium that causes meliodosis in humans and animals. Meliodosis is a disease found in certain parts of Asia, Thailand, and Australia. B. pseudomallei is typically a soil organism and has been recovered from rice paddies and moist tropical soil, but as an opportunistic pathogen can cause disease in susceptible subjects such as those that suffer from diabetes mellitus. The organism can exist intracellularly, and causes pneumonia and bacteremia (spread of the bacterium through the bloodstream). The latency period can be extremely long, with infection preceding disease by decades, and treatment can take months of antibiotic use, with relapse a commonly observed phenomenon. Intercellular spread can occur via induction of actin polymerization at one pole of the cell, allowing movement through the cytoplasm and from cell-to-cell. This organism carries a number of small sequence repeats which may promoter antigenic variation, similar to what was found with the B. mallei genome.

Burkholderia cepacia is a Gram-negative bacterium composed of at least seven different sub-species, including Burkholderia multivorans, Burkholderia vietnamiensis, Burkholderia stabilis, Burkholderia cenocepacia and Burkholderia ambifaria. B. cepacia is an important human pathogen which most often causes pneumonia in people with underlying lung disease (such as cystic fibrosis or immune problems (such as (chronic granulomatous disease). B. cepacia is typically found in water and soil and can survive for prolonged periods in moist environments. Person-to-person spread has been documented; as a result, many hospitals, clinics, and camps for patients with cystic fibrosis have enacted strict isolation precautions B. cepacia. Subjects with the bacteria are often treated in a separate area than those without to limit spread. This is because infection with B. cepacia can lead to a rapid decline in lung function resulting in death. Diagnosis of B. cepacia involves isolation of the bacteria from sputum cultures. Treatment is difficult because B. cepacia is naturally resistant to many common antibiotics including aminoglycosides (such as tobramycin) and polymixin B. Treatment typically includes multiple antibiotics and may include ceftazidime, doxycycline, piperacillin, chloramphenicol, and co-trimoxazole.

Francisella tularensis was first noticed as the causative agent of a plague-like illness that affected squirrels in Tulare County in California in the early part of the 20th century by Edward Francis. The organism now bears his namesake. The disease is called tularemia and has been noted throughout recorded history. The organism can be transmitted from infected ticks or deerflies to a human, through infected meat, or via aerosol, and thus is a potential bioterrorism agent. It is an aquatic organism, and can be found living inside protozoans, similar to what is observed with Legionella. It has a high infectivity rate, and can invade phagocytic and nonphagocytic cells, multiplying rapidly. Once within a macrophage, the organism can escape the phagosome and live in the cytosol.

The bacterial infection may also be an infection with Porphyromonas gingivalis. P. gingivalis is a gram-negative, anaerobic bacterium that is implicated in the pathogenesis of periodontal disease, and is also found in the upper gastrointestinal tract, respiratory tract and the colon. Pathogenesis of P. gingivalis is known to involve the recruitment of activators of plasminogen.

The invention also finds use in veterinary applications. A healthy microflora in the gastro-intestinal tract of livestock is of vital importance for health and corresponding production of associated food products. As with humans, the gastrointestinal tract of a healthy animal contains numerous types of bacteria (i.e., E. coli, Pseudomonas aeruginosa and Salmonella spp.), which live in ecological balance with one another. This balance may be disturbed by a change in diet, stress, or in response to antibiotic or other therapeutic treatment, resulting in bacterial diseases in the animals generally caused by bacteria such as Salmonella, Campylobacter, Enterococci, Tularemia and E. coli. Bacterial infection in these animals often necessitates therapeutic intervention, which has treatment costs as well-being frequently associated with a decrease in productivity.

The term “treat”, “treating” or “treatment” as used herein also refers to administering compositions or one or more of pharmaceutically active ingredients discussed herein, with or without additional pharmaceutically active or inert ingredients, in order to: (i) reduce or eliminate either a bacterial infection or one or more symptoms of the bacterial infection, or (ii) retard the progression of a bacterial infection or of one or more symptoms of the bacterial infection, or (iii) reduce the severity of a bacterial infection or of one or more symptoms of the bacterial infections, or (iv) suppress the clinical manifestation of a bacterial infection, or (v) suppress the manifestation of adverse symptoms of the bacterial infections. Further, the terms “treating” and “treatment” may include one or more of, ameliorating a symptom of a bacterial infection in a subject, blocking or ameliorating a recurrence of a symptom of a bacterial infection in a subject, decreasing in severity and/or frequency a symptom of a bacterial infection in a subject, stasis, decreasing, or inhibiting growth of a vegetative form of bacteria in a subject, inhibiting bacterial sporulation in a subject, inhibiting activation of a bacterial spore in a subject, inhibiting germination of a bacterial spore in a subject, and inhibiting outgrowth of a bacterial spore in a subject. Treatment means ameliorating, blocking, reducing, decreasing or inhibiting by about 1% to about 100% versus a subject to which an antigen binding protein or composition of the present invention has not been administered. Preferably, the ameliorating, blocking, reducing, decreasing or inhibiting is 100%, 99%, 98%, 97%, 96%, 95%, 90%, 80%, 70%, 60%, 50%, 40%, 30%, 20%, 10%, 5% or 1% versus a subject to which an antigen binding protein or composition of the present invention has not been administered.

Successful treatment may generally mean improvement in any symptoms associated with or caused by a Gram-positive or Gram-negative bacterial infection and for example may refer to an improvement in any of the following: fever, inflammation, swelling, vomiting, fatigue, cramping, coughing, sneezing, respiratory illness, diarrhea, meningitis, headaches, joint pain, body aches, blisters, rashes, nausea, chills, dizziness, drowsiness, sleeplessness, gagging, skin irritation, excessive mucus production (e.g. in the eyes, gastrointestinal tract, sinuses, or respiratory system), ulcers, gastrointestinal discomfort, skin loss, hair loss, necrosis, and organ dysfunction. Improvements in any of these symptoms or in the bacterial infection or conditions described herein can be readily assessed according to standard methods and techniques known in the art. The population of subjects treated by the method of the disease includes subjects suffering from the undesirable condition or disease, as well as subjects at risk for development of the condition or disease.

As used herein, the terms “inhibit”, “inhibiting” and “inhibition” have their ordinary and customary meanings, and include one or more of inhibiting growth or a function of bacteria, inhibiting growth of a vegetative form of bacteria, inhibiting a function of a vegetative form of bacteria, inhibiting propagation of bacteria, inhibiting bacterial sporulation, inhibiting activation of a bacterial spore, inhibiting germination of a bacterial spore, and inhibiting outgrowth of a bacterial spore. Such inhibition is an inhibition of about 1% to about 100% of the particular activity versus the activity in a subject to which an antigen binding protein or composition of the present invention has not been administered. Preferably, the inhibition is an inhibition of 100%, 99%, 98%, 97%, 96%, 95%, 90%, 80%, 70%, 60%, 50%, 40%, 30%, 20%, 10%, 5% or 1% of the activity versus a subject to which an antigen binding protein or composition of the present invention has not been administered. As used herein, “spore” refers to both the conventionally used terms “spore” and “endospore.”

As used herein, the terms “preventing” and “prevention” have their ordinary and customary meanings, and includes one or more of preventing colonization of bacteria in a subject, preventing an increase in the growth of a population of bacteria in a subject, preventing activation, germination or outgrowth of bacterial spores in a subject, preventing sporulation of bacteria in a subject, preventing development of a disease caused by bacteria in a subject, and preventing symptoms of a disease caused by bacteria in a subject. As used herein, the prevention lasts at least about 0.5, 1, 2, 3, 4, 5, 6, 7, 10, 12, 15, 20, 25, 30, 35, 40 or more days after administration of an antigen binding protein or composition of the present invention.

As used herein, “prophylaxis” includes inhibiting the development of a productive or progressive infection by bacteria in a subject, where the prophylaxis lasts at least about 0.5, 1, 2, 3, 4, 5, 6, 7, 10, 12, 15, 20, 25, 30, 35, 40 or more days after administration of an antigen binding protein or composition of the present invention. Inhibition against development of a productive or progressive infection by a bacterium means that the severity of an infection in a subject is reduced by about 1% to about 100% versus a subject to which an antigen binding protein or composition of the present invention has not been administered. Preferably, the reduction in severity is a 100%, 99%, 98%, 97%, 96%, 95%, 90%, 80%, 70%, 60%, 50%, 40%, 30%, 20%, 10%, 5% or 1% reduction in severity. The severity of an infection may be based on the amount of bacteria present in a subject, the length of time that the bacteria can be detected in a subject, and/or the severity of a symptom of a bacterial infection, among other factors.

Compositions

In some examples, an antigen binding protein as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an antigen binding protein into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding protein dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding protein of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Non-aqueous vehicles such as mixed oils and ethyl oleate may also be used. Liposomes may also be used as carriers. The vehicles may contain minor amounts of additives that enhance isotonicity and chemical stability, e.g., buffers and preservatives.

The antigen binding proteins of the present invention may be formulated for local or topical administration, such as for topical application to the skin or tissue requiring treatment. Formulations for topical administration typically comprise a topical vehicle combined with active agent(s), with or without additional optional components. The pharmaceutical compositions of the invention may be in the form of a spray, cream, gel, lotion or the like for topical administration.

Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerine), glycols such as butylene, isoprene or propylene glycol, aliphatic alcohols such as lanolin, mixtures of water and organic solvents and mixtures of organic solvents such as alcohol and glycerine, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity. Both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, may also contain solvents, emulsifiers, moisturizers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturizers, emollients, fragrances, dyes/colorants, preservatives and other active ingredients that increase or enhance the efficacy of the final product.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxyethylcellulose, xanthan gum, magnesium aluminium silicate, silica, microcrystalline wax, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylceilulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used, and compositions may be formulated for transdermal administration (for example, as a transdermal patch).

Upon formulation, an antigen binding protein of the present invention will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding protein of the present invention.

WO2002/080967 describes compositions and methods for administering aerosolized compositions comprising antibodies for the treatment of, e.g., asthma, which are also suitable for administration of an antigen binding protein of the present invention.

Dosages and Timing of Administration

Suitable dosages of an antigen binding protein of the present invention will vary depending on the specific an antigen binding protein, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma may be measured, for example, by high performance liquid chromatography.

In some examples, a method of the present invention comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding protein(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding protein(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding protein(s), rather the present invention encompasses any amount of the antigen binding protein(s) sufficient to achieve the stated result in a subject.

Kits

The present invention additionally comprises a kit comprising one or more of the following:
(i) an antigen binding protein of the invention or expression construct(s) encoding same;
(ii) a cell of the invention;
(iii) a complex of the invention; or
(iii) a pharmaceutical composition of the invention.

In the case of a kit for detecting plasminogen, the kit can additionally comprise a detection means, e.g., linked to an antigen binding protein of the invention.

In the case of a kit for therapeutic/prophylactic use, the kit can additionally comprise a pharmaceutically acceptable carrier.

Optionally a kit of the invention is packaged with instructions for use in a method described herein according to any example.

It will be understood that the invention disclosed and defined in this specification extends to all alternative combinations of two or more of the individual features mentioned or evident from the text or drawings. All of these different combinations constitute various alternative aspects of the invention.

TABLE 1
Summary of amino acid and nucleotide sequences

| Antibody ID | Region | SEQ ID NO: | Amino acid or nucleotide sequence |
|---|---|---|---|
| G05 | LCDR1 | 1 | GRSSY |
| | (protein) | | |
| | LCDR2 | 2 | DNT |
| | (protein) | | |
| | LCDR3 | 3 | GSADSSTTGIFG |
| | (protein) | | |
| | HCDR1 | 4 | GFTFSSYA |
| | (protein) | | |
| | HCDR2 | 5 | IDNIGSYT |
| | (protein) | | |
| | HCDR3 | 6 | AKSAYGGSCCGPAYIDA |
| | (protein) | | |
| | VL | 7 | QAALTQPSSVSANPGETVEITCSGGGRSSYYGWYQQKSPGSAPVTL |
| | (protein) | | IYDNTNRPSNIPSRFSGSKSGSTATLTITGVQGEDEAVYFCGSADS |
| | | | STTGIFGAGTTLTVL |
| | VH | 8 | AVTLDESGGGLQTPGRALSLVCKASGFTFSSYAMHWVRQAPGKGLE |
| | (protein) | | FVAGIDNIGSYTRYGAAVKGRVTISRDNGQSIVRLQLDNLRAEDTG |
| | | | TYYCAKSAYGGSCCGPAYIDAWGHGTEVIVSS |
| | LCDR1 | 9 | GGACGGTCATCCTAT |
| | (DNA) | | |
| | LCDR2 | 10 | GACAACACA |
| | (DNA) | | |
| | LCDR3 | 11 | GGCAGCGCCGATAGCTCCACCACAGGCATCTTTGGA |
| | (DNA) | | |
| | HCDR1 | 12 | GGCTTCACATTTTCTAGCTATGCC |
| | (DNA) | | |
| | HCDR2 | 13 | ATCGACAATATCGGCTCCTACACC |
| | (DNA) | | |
| | HCDR3 | 14 | GCAAAGAGCGCCTACGGCGGCTCCTGCTGTGGACCAGCATATATCG |
| | (DNA) | | ACGCA |
| | VL (DNA) | 15 | CAGGCGGCCCTGACTCAGCCCTCTTCTGTCTCTGCCAATCCAGGCG |
| | | | AAACTGTCGAAATTACTTGTTCCGGGGGGGGACGGTCATCCTATTA |
| | | | CGGGTGGTATCAGCAGAAGTCTCCCGGCAGCGCCCCTGTGACCCTG |
| | | | ATCTATGACAACACAAATAGGCCATCCAACATCCCCTCTCGCTTCT |
| | | | CCGGCTCTAAGAGCGGCTCCACCGCCACACTGACCATCACAGGAGT |
| | | | GCAGGGAGAGGACGAGGCCGTGTACTTCTGCGGCAGCGCCGATAGC |
| | | | TCCACCACAGGCATCTTTGGAGCAGGCACCACACTGACCGTGCTG |
| | VH (DNA) | 16 | GCCGTGACCCTGGATGAGTCCGGAGGAGGCCTCCAGACACCTGGCC |
| | | | GGGCCCTGAGCCTGGTGTGCAAGGCCAGCGGCTTCACATTTTCTAG |
| | | | CTATGCCATGCACTGGGTGAGGCAGGCCCCAGGCAAGGGCCTGGAG |
| | | | TTTGTGGCCGGCATCGACAATATCGGCTCCTACACCAGATATGGAG |
| | | | CCGCCGTGAAGGGAAGGGTGACAATCTCCAGAGATAACGGCCAGTC |
| | | | TATCGTGCGGCTCCAGCTGGACAATCTGAGAGCCGAGGATACCGGC |
| | | | ACATACTATTGCGCAAAGAGCGCCTACGGCGGCTCCTGCTGTGGAC |
| | | | CAGCATATATCGACGCATGGGGCCACGGCACCGAGGTGATCGTGTC |
| | | | ATCC |
| | LFR1 | 17 | QAALTQPSSVSANPGETVEITCSGG |
| | (protein) | | |
| | LFR2 | 18 | YGWYQQKSPGSAPVTLIY |
| | (protein) | | |
| | LFR3 | 19 | NRPSNIPSRFSGSKSG STATLTITGVQGEDEAVYFC |
| | (protein) | | |
| | LFR4 | 20 | AGTTLTVL |
| | (protein) | | |
| | HFR1 | 21 | AVTLDESGGGLQTPGRALSLVCKAS |
| | (protein) | | |
| | HFR2 | 22 | MHWWRQAPGKGLEFVAG |
| | (protein) | | |
| | HFR3 | 23 | RYGAAVKGRVTISRDNGQSIVRLQLDNLRAEDTGTYYC |
| | (protein) | | |
| | HFR4 | 24 | WGHGTEVIVSS |
| | (protein) | | |
| | LFR1 | 25 | CAGGCGGCCCTGACTCAGCCCTCTTCTGTCTCTGCCAATCCAGGCG |
| | (DNA) | | AAACTGTCGAAATTACTTGTTCCGGGGGG |
| | LFR2 | 26 | TACGGGTGGTATCAGCAGAAGTCTCCCGGCAGCGCCCCTGTGACCC |
| | (DNA) | | TGATCTAT |
| | LFR3 | 27 | AATAGGCCATCCAACATCCCCTCTCGCTTCTCCGGCTCTAAGAGCG |
| | (DNA) | | GCTCCACCGCCACACTGACCATCACAGGAGTGCAGGGAGAGGACGA |
| | | | GGCCGTGTACTTCTGC |
| | LFR4 | 28 | GCAGGCACCACACTGACCGTGCTG |
| | (DNA) | | |
| | HFR1 | 29 | GCCGTGACCCTGGATGAGTCCGGAGGAGGCCTCCAGACACCTGGCC |
| | (DNA) | | GGGCCCTGAGCCTGGTGTGCAAGGCCAGC |
| | HFR2 | 30 | ATGCACTGGGTGAGGCAGGCCCCAGGCAAGGGCCTGGAGTTTGTGG |
| | (DNA) | | CCGGC |
| | HFR3 | 31 | AGATATGGAGCCGCCGTGAAGGGAAGGGTGACAATCTCCAGAGATA |
| | (DNA) | | ACGGCCAGTCTATCGTGCGGCTCCAGCTGGACAATCTGAGAGCCGA |
| | | | GGATACCGGCACATACTATTGC |
| | HFR4 | 32 | TGGGGCCACGGCACCGAGGTGATCGTGTCATCC |
| | (DNA) | | |
| G11 | LCDR1 | 33 | GTY |
| | (protein) | | |
| | LCDR2 | 34 | QNN |
| | (protein) | | |
| | LCDR3 | 35 | GGYDSSAGYATFG |
| | (protein) | | |
| | HCDR1 | 36 | GFSISSYS |
| | (protein) | | |
| | HCDR2 | 37 | ISSDGSDT |
| | (protein) | | |
| | HCDR3 | 38 | AKPTGGYYTWYETGSIDT |
| | (protein) | | |
| | VL | 39 | QAALTQPSSVSANPGEIVEITCSGGGTYYGWYQQKSPGSAPVTMIY |
| | (protein) | | QNNQRPSNIPSRFSGSGSGPANTLTITGVRAEDEAVYYCGGYDSSA |
| | | | GYATFGAGTTLTVL |
| | VH | 40 | AVTLDESGGGLQTPGGGLSLVCKASGFSISSYSMYWVRQAPGKGLE |
| | (protein) | | FVASISSDGSDTSYGSAVKGRATISRDNGQSTVRLQLNYLRAEDTG |
| | | | TYFCAKPTGGYYTWYETGSIDTWGHGTEVIVSS |
| | LCDR1 | 41 | GGCACCTAC |
| | (DNA) | | |
| | LCDR2 | 42 | CAGAACAAT |
| | (DNA) | | |
| | LCDR3 | 43 | GGCGGCTACGATAGCTCCGCCGGCTATGCCACATTTGGC |
| | (DNA) | | |
| | HCDR1 | 44 | GGCTTCAGCATCTCTAGCTACTCC |
| | (DNA) | | |
| | HCDR2 | 45 | ATCTCCTCTGACGGCTCCGATACA |
| | (DNA) | | |
| | HCDR3 | 46 | GCCAAGCCCACCGGCGGCTACTATACATGGTACGAGACAGGCTCCA |
| | (DNA) | | TCGATACA |
| | VL (DNA) | 47 | CAGGCGGCCCTGACTCAGCCTTCTTCTGTCTCCGCCAACCCCGGCG |
| | | | AGATCGTGGAGATCACATGCTCTGGCGGCGGCACCTACTATGGCTG |
| | | | GTATCAGCAGAAGAGCCCAGGCTCCGCCCCTGTGACCATGATCTAT |
| | | | CAGAACAATCAGAGGCCATCCAACATCCCCTCTCGCTTCAGCGGCT |
| | | | CCGGCTCTGGACCTGCAAATACCCTGACAATCACCGGCGTGAGGGC |
| | | | AGAGGACGAGGCCGTGTACTATTGCGGCGGCTACGATAGCTCCGCC |
| | | | GGCTATGCCACATTTGGCGCAGGCACCACACTGACCGTGCTG |
| | VH (DNA) | 48 | GCCGTGACACTGGACGAGAGCGGAGGAGGCCTGCAGACCCCTGGAG |
| | | | GAGGCCTGAGCCTGGTGTGCAAGGCCTCTGGCTTCAGCATCTCTAG |
| | | | CTACTCCATGTATTGGGTGCGGCAGGCACCAGGCAAGGGCCTGGAG |
| | | | TTTGTGGCCAGCATCTCCTCTGACGGCTCCGATACATCTTACGGCA |
| | | | GCGCCGTGAAGGGAAGGGCAACAATCAGCAGAGACAACGGCCAGTC |
| | | | CACCGTGCGGCTGCAGCTGAATTACCTGAGAGCCGAGGATACAGGC |
| | | | ACCTATTTCTGTGCCAAGCCCACCGGCGGCTACTATACATGGTACG |
| | | | AGACAGGCTCCATCGATACATGGGGCCACGGCACCGAAGTGATCGT |
| | | | CTCATCA |
| | LFR1 | 49 | QAALTQPSSVSANPGEIVEITCSGG |
| | (protein) | | |
| | LFR2 | 50 | YGWYQQKSPGSAPVTMIY |
| | (protein) | | |
| | LFR3 | 51 | QRPSNIPSRFSGSGSGPANTLTITGVRAEDEAVYYC |
| | (protein) | | |
| | LFR4 | 52 | AGTTLTVL |
| | (protein) | | |
| | HFR1 | 53 | AVTLDESGGGLQTPGGGLSLVCKAS |
| | (protein) | | |
| | HFR2 | 54 | MYWWRQAPGKGLEFVAS |
| | (protein) | | |
| | HFR3 | 55 | SYGSAVKGRATISRDNGQSTVRLQLNYLRAEDTGTYFC |
| | (protein) | | |
| | HFR4 | 56 | WGHGTEVIVSS |
| | (protein) | | |
| | LFR1 | 57 | CAGGCGGCCCTGACTCAGCCTTCTTCTGTCTCCGCCAACCCCGGCG |
| | (DNA) | | AGATCGTGGAGATCACATGCTCTGGCGGC |
| | LFR2 | 58 | TATGGCTGGTATCAGCAGAAGAGCCCAGGCTCCGCCCCTGTGACCA |
| | (DNA) | | TGATCTAT |
| | LFR3 | 59 | CAGAGGCCATCCAACATCCCCTCTCGCTTCAGCGGCTCCGGCTCTG |
| | (DNA) | | GACCTGCAAATACCCTGACAATCACCGGCGTGAGGGCAGAGGACGA |
| | | | GGCCGTGTACTATTGC |
| | LFR4 | 60 | GCAGGCACCACACTGACCGTGCTG |
| | (DNA) | | |
| | HFR1 | 61 | GCCGTGACACTGGACGAGAGCGGAGGAGGCCTGCAGACCCCTGGAG |
| | (DNA) | | GAGGCCTGAGCCTGGTGTGCAAGGCCTCT |
| | HFR2 | 62 | ATGTATTGGGTGCGGCAGGCACCAGGCAAGGGCCTGGAGTTTGTGG |
| | (DNA) | | CCAGC |
| | HFR3 | 63 | TCTTACGGCAGCGCCGTGAAGGGAAGGGCAACAATCAGCAGAGACA |
| | (DNA) | | ACGGCCAGTCCACCGTGCGGCTGCAGCTGAATTACCTGAGAGCCGA |
| | | | GGATACAGGCACCTATTTCTGT |
| | HFR4 | 64 | TGGGGCCACGGCACCGAAGTGATCGTCTCATCA |
| | (DNA) | | |
| hPIg | | 65 | EPLDDYVNTQGASLFSVTKKQLGAGSIEECAAKCEEDEEFTCRAFQ |
| (mature sequence | | | YHSKEQQCVIMAENRKSSIIIRMRDVVLFEKKVYLSECKTGNGKNY |
| shown with | | | RGTMSKTKNGITCQKWSSTSPHRPRFSPATHPSEGLEENYCRNPDN |
| signal peptide | | | DPQGPWCYTTDPEKRYDYCDILECEEECMHCSGENYDGKISKTMSG |
| removed) | | | LECQAWDSQSPHAHGYIPSKFPNKNLKKNYCRNPDRELRPWCFTTD |
| | | | PNKRWELCDIPRCTTPPPSSGPTYQCLKGTGENYRGNVAVTVSGHT |
| | | | CQHWSAQTPHTHNRTPENFPCKNLDENYCRNPDGKRAPWCHTTNSQ |
| | | | VRWEYCKIPSCDSSPVSTEQLAPTAPPELTPVVQDCYHGDGQSYRG |
| | | | TSSTTTTGKKCQSWSSMTPHRHQKTPENYPNAGLTMNYCRNPDADK |
| | | | GPWCFTTDPSVRWEYCNLKKCSGTEASVVAPPPVVLLPDVETPSEE |
| | | | DCMFGNGKGYRGKRATTVTGTPCQDWAAQEPHRHSIFTPETNPRAG |
| | | | LEKNYCRNPDGDVGGPWCYTTNPRKLYDYCDVPQCAAPSFDCGKPQ |
| | | | VEPKKCPGRVVGGCVAHPHSWPWQVSLRTRFGMHFCGGTLISPEWV |
| | | | LTAAHCLEKSPRPSSYKVILGAHQEVNLEPHVQEIEVSRLFLEPTR |
| | | | KDIALLKLSSPAVITDKVIPACLPSPNYVVADRTECFITGWGETQG |
| | | | TFGAGLLKEAQLPVIENKVCNRYEFLNGRVQSTELCAGHLAGGTDS |
| | | | CQGDSGGPLVCFEKDKYILQGVTSWGLGCARPNKPGVYVRVSRFVT |
| | | | WIEGVMRNN |
| hPIg | | 66 | EPKKCPGRVVGGCVAH |
| activation loop | | | |
| (E554 to H569, | | | |
| with residues | | | |
| Arg561 and | | | |
| Valine 562 | | | |
| shown in bold) | | | |

TABLE 2
Key interactions of G05/KR5-SP. Interactions involving KR5 and
SP residues are highlighted in italics and bold respectively.

| | | |
|---|---|---|
| G05 residue | Distance (Å) | KR5-SP residue |

G05(chain B):KR5-SP(chain C)

| | | |
|---|---|---|
| ASN 213 [OD1] | 3.82 | C:ARG 493 [NE] |
| ASN 213 [ND2] | 3.24 | C:ARG 493 [O] |
| ASP 212 [OD1] | 2.41 | C:SER 495 [OG] |
| ASP 212 [OD2] | 2.86 | C:SER 495 [N] |
| ASP 212 [OD2] | 3.32 | C:ILE 496 [N] |
| THR 186 [OG1] | 3.13 | C:ASP 516 [O] |
| LYS 193 [NZ] | 3.55 | C:ASP 516 [OD2] |
| THR 186 [N] | 3.21 | C:GLY 517 [O] |
| LYS 193 [NZ] | 2.62 | C:ASP 518 [OD2] |
| THR 186 [O] | 3.02 | C:VAL 519 [N] |
| GLY 194 [O] | 2.71 | C:TYR 525 [OH] |
| ALA 190 [O] | 3.75 | C:TYR 533 [OH] |
| SER 232 [OG] | 2.53 | C:GLU 554 [OE1] |
| ARG 187 [NH2] | 2.87 | C:GLU 554 [OE1] |
| CYS 233 [N] | 3.64 | C:GLU 554 [OE1] |
| SER 232 [N] | 2.81 | C:GLU 554 [OE2] |
| SER 96 [OG] | 2.85 | C:GLU 554 [N] |
| ASP 52 [OD2] | 3.69 | C:LYS 556 [NZ] |
| GLY 231 [N] | 2.85 | C:LYS 556 [O] |
| GLY 230 [N] | 3.23 | C:LYS 556 [O] |
| GLY 230 [N] | 3.02 | C:LYS 557 [O] |
| ASN 181 [OD1] | 2.73 | C:LYS 557 [NZ] |
| TYR 238 [OH] | 2.66 | C:HIS 569 [NE2] |
| TYR 185 [OH] | 3.62 | C:ASP 751 [O] |

TABLE 3
Key interactions of G05/KR5-SP. Interactions involving
KR5 and SP residues are highlighted in italics and
bold respectively (data derived from the second complex
pair in the crystallographic asymmetric unit).

| | | |
|---|---|---|
| G05 residue | Distance (Å) | KR5-SP residue |

G05(chain A):KR5-SP(chain D)

| | | |
|---|---|---|
| SER 184 [O] | 3.50 | LYS 468 [NZ] |
| ASN 213 [ND2] | 3.11 | ARG 493 [O] |
| ASP 212 [OD2] | 2.85 | SER 495 [N] |
| ASP 212 [OD1] | 2.25 | SER 495 [OG] |
| ASP 212 [OD2] | 3.79 | ILE 496 [N] |
| LYS 193 [NZ] | 2.55 | ASP 516 [OD2] |
| THR 186 [OG1] | 3.89 | GLY 517 [N] |
| GLY 194 [O] | 2.73 | TYR 525 [OH] |
| SER 232 [OG] | 2.61 | GLU 554 [OE1] |
| ARG 187 [NE] | 3.34 | GLU 554 [OE1] |
| CYS 233 [N] | 3.60 | GLU 554 [OE1] |
| ARG 187 [NH2] | 3.06 | GLU 554 [OE1] |
| SER 232 [N] | 2.83 | GLU 554 [OE2] |
| GLY 231 [N] | 2.85 | LYS 556 [O] |
| GLY 230 [N] | 3.25 | LYS 556 [O] |
| ASP 52 [OD2] | 3.71 | LYS 556 [NZ] |
| GLY 230 [N] | 3.04 | LYS 557 [O] |
| ASN 181 [OD1] | 2.86 | LYS 557 [NZ] |
| TYR 160 [OH] | 3.88 | ARG 561 [NE] |
| TYR 238 [OH] | 2.71 | HIS 569 [NE2] |

TABLE 4
Key interactions of G11/KR5-SP.

| G11 residue | Distance (Å) | K5-SP residue |
|---|---|---|
| ASP 181 [OD1] | 3.69 | ARG 677 [NE] |
| ASP 181 [OD1] | 3.12 | ARG 677 [NH2] |
| ASP 181 [OD2] | 2.79 | ARG 677 [NH2] |
| TYR 234 [OH] | 2.88 | ARG 712 [NH1] |
| GLY 96 [O] | 3.58 | ASN 769 [ND2] |
| TYR 231 [OH] | 2.47 | ASP 676 [OD1] |
| TYR 231 [OH] | 3.77 | VAL 704 [O] |
| TYR 234 [OH] | 2.48 | GLU 714 [OE1] |
| THR 30 [N] | 2.96 | GLU 714 [OE2] |
| THR 30 [OG1] | 2.57 | GLU 714 [OE2] |

### EXAMPLES

### Example 1: Production of G05 and G11 Antibodies

Antibodies for binding to plasminogen were obtained by raising an antibody response in chickens to full length plasminogen. Antibody variable heavy and light chains (VH and VL) were amplified from cDNA from chicken spleen and linked via a flexible linker to create an scFv library.

Selection was performed by screening for plasminogen-binding antibodies via Biacore. SK-mediated plasminogen activation and fibrinolytic assays were used to identify antibodies which prevented or inhibited activation of plasminogen to plasmin.

### Example 2: Characterisation of Antibodies G05 and G11 for Binding Plasminogen Activation Loop

SPR Assays

Chicken antibodies were immobilised on a series S CM4 (GE healthcare) chip through amine coupling. The binding of plasminogen or plasmin to antibodies of the invention, at concentrations ranging from 0.39 nM to as high as 50 nM was analysed using Biacore T200 (GE Healthcare) in a buffer composed of 10 mM HEPES pH 7.4, 150 mM NaCl, 3 mM EDTA and 0.05% Tween20 in the presence of protease inhibitor cocktail. Plasminogen/plasmin was injected at 30 μl/min for 180 s association, followed by a 600 s dissociation. At the end of each cycle, the sensor chip was regenerated with 10 mM glycine pH 1.8 before the next injection, a minimum of 6 cycles was performed. To obtain kinetic parameters, sensorgrams were fitted with a Langmuir 1:1 binding model using Biacore T200 evaluation software (GE Healthcare).

Binding to Plasminogen or Plasmin on CM5 Chip

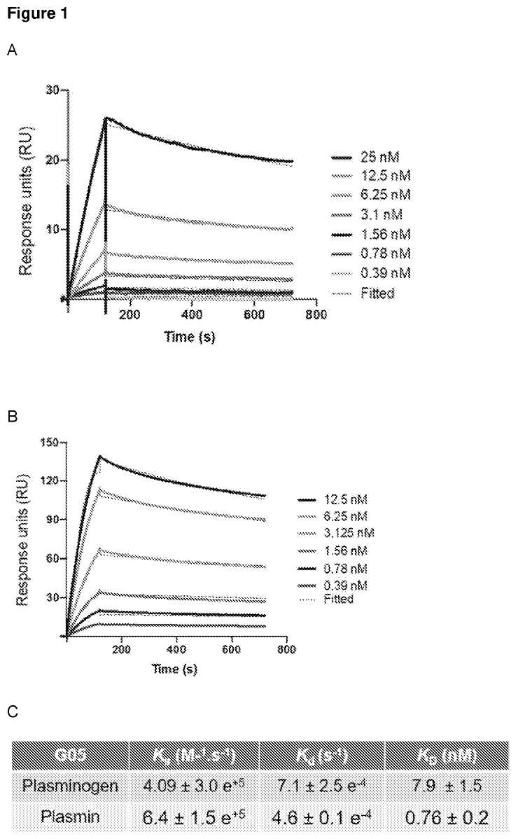

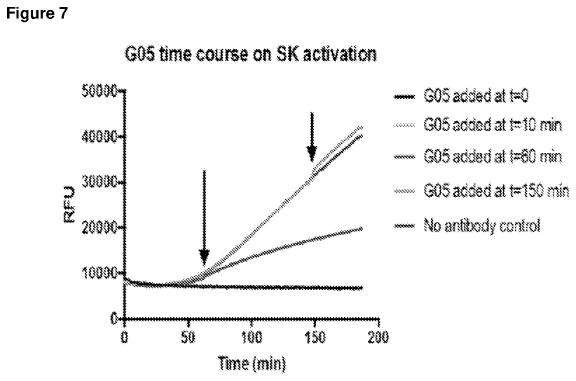

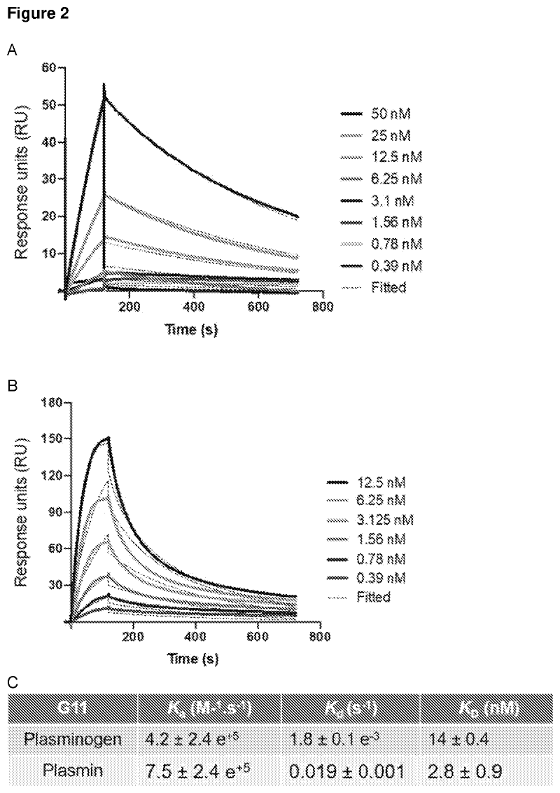

The results of the binding experiments (FIGS. 1 and 2), show that an antigen binding protein of the invention binds to both plasminogen and plasmin, but with approximately 10-fold greater affinity for plasminogen compared to plasmin.

Inhibition of tPA-Mediated Plasminogen Activation in Solution and on Fibrin

In Solution:

20 nM of plasminogen was mixed with various G05 and G11 concentrations (0-200 nM) in the presence of 20 mM EACA for 30 minutes at room temperature. After incubation, plasminogen activation by 4 nM of tPA was measured using fluorogenic substrate (H-Ala-Phe-Lys-AMC, Bachem) in a Fluostar Omega plate reader (BMG Labtech), excitation and emission wavelengths of 355 nm and 460 nm, respectively. The progress curves were fitted to a non-linear exponential equation in GraphPad Prism 6:

Y=Y₀*exp(rate of activation*X)

where Y₀ is the Y-value when X=0. The rates of activation were plotted against corresponding G05 or G11 concentration to yield an inhibition curve that can be fitted with the inhibitor vs response model in GraphPad Prism 6:

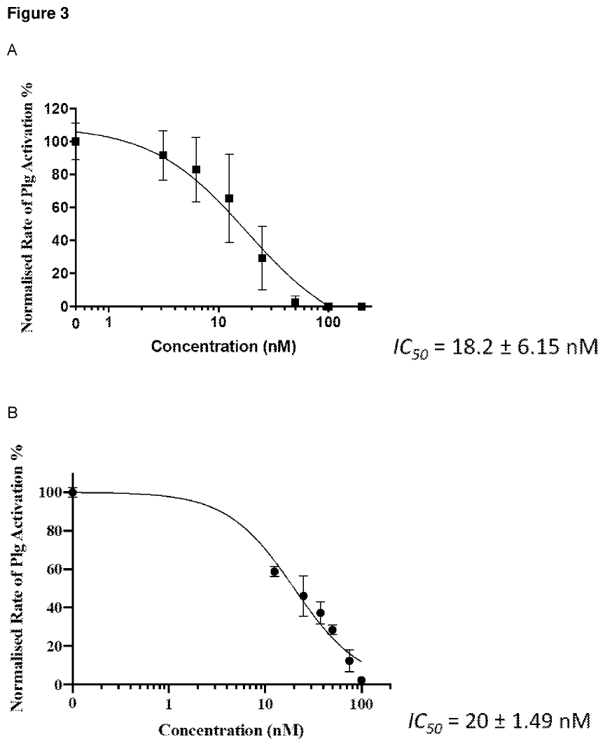

Y=Bottom+(Top−Bottom)/(1+((X^{HillSlope})/(IC₅₀^{HillSlope})))

where Top and Bottom are plateaus in the fluorescence reading and Hillslope is a measure of the steepness of the curves. The IC₅₀ value, i.e., the concentration of G05 that inhibits 50% of tPA-mediated plasminogen activation in solution, is 17.29±2.73 nM (FIG. 3A). The IC₅₀ value for G11 is 20 nM (FIG. 3B).

On Fibrin:

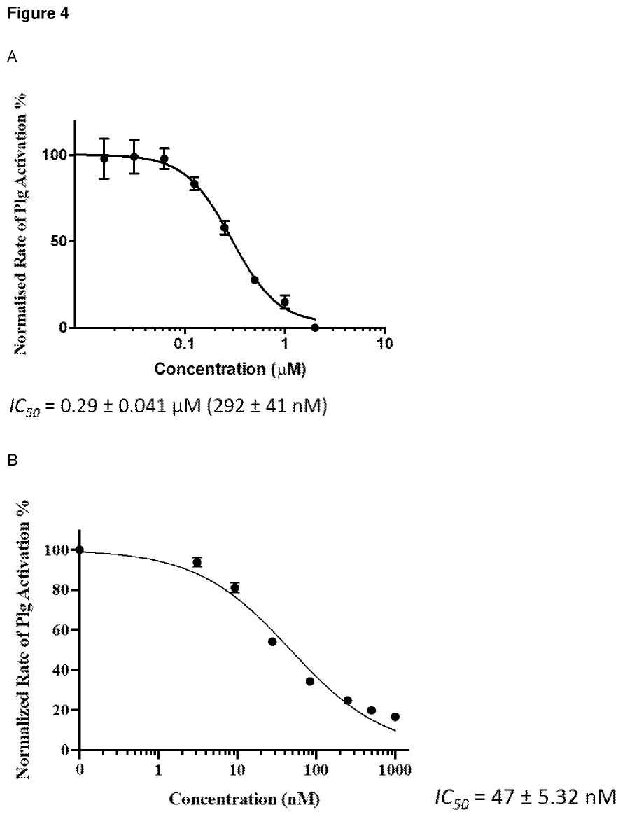

Plasminogen activation was measured on the surface of the preformed fibrin clot, prepared by mixing 3 mg/ml fibrinogen (Banksia Scientific); 1 U of bovine thrombin (Jomar Life Research); and 10 nM of tPA (Boehringer Ingelheim), at 37° C. for 2 hours. 100 nM of plasminogen, mixed with G05 or G11 at concentrations (0-2 μM), was added to the surface of the clot. Plasmin activity was monitored using 200 μM of fluorogenic substrate (H-Ala-Phe-Lys-AMC, Bachem) as above. The rate of plasminogen activation and IC₅₀ were calculated as above. The IC₅₀ value obtained for the inhibition of tPA-mediated plasminogen activation on fibrin by G05 is 292 nM (FIG. 4A). The IC₅₀ value obtained for the inhibition of tPA-mediated plasminogen activation on fibrin by G11 is 47 nM (FIG. 4B).

Non-Inhibition of Plasmin

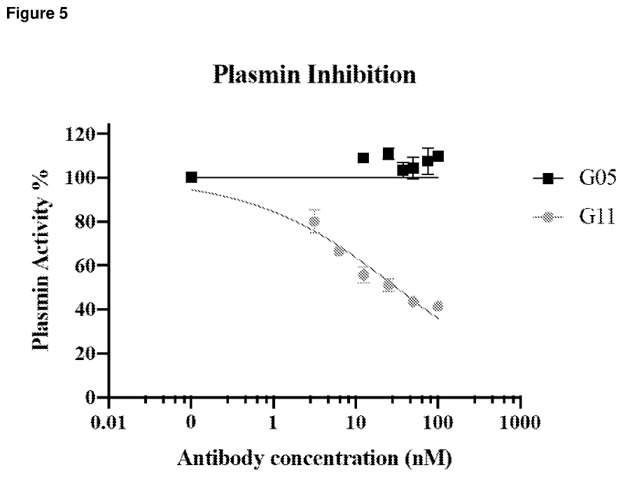

Plasmin (Haematologic Technologies) activity was measured in the presence of 200 μM of fluorogenic substrate (H-Ala-Phe-Lys-AMC, Bachem) in a Fluostar Omega plate reader (BMG Labtech) via excitation and emission wavelengths of 355 nm and 460 nm respectively. Progress curves of G05 and A01 (a non-inhibitory plasminogen antibody) at 10:1 antibody:plasmin ratio were obtained. G05 showed slight agonistic effect on Plm activity compared to A01. 20 nM of plasmin and the antibody G05 (0-200 nM) at 37° C. using a Fluostar Omega plate reader (BMG Labtech) via excitation and emission wavelengths of 355 nm and 460 nm, respectively. A weak G05 concentration-dependent agonistic effect was observed (FIG. 5).

Inhibition of Streptokinase Binding to Plasminogen by G05

The impact of G05 on binding of streptokinase to plasminogen was investigated using Biacore T200 (GE Healthcare). 10 nM plasminogen was passed over streptokinase immobilized on a CM4 (GE Healthcare) chip in the presence of G05 or a naïve chicken antibody (gAb), at 0, 62.5, 125, 250 and 500 nM. G05 showed inhibition at 125 nM and above. Naïve chicken antibody gAb, the control, showed no inhibition. Percentage of SK binding in the presence of 500 nM G05 and gAb, normalized against no antibody control.

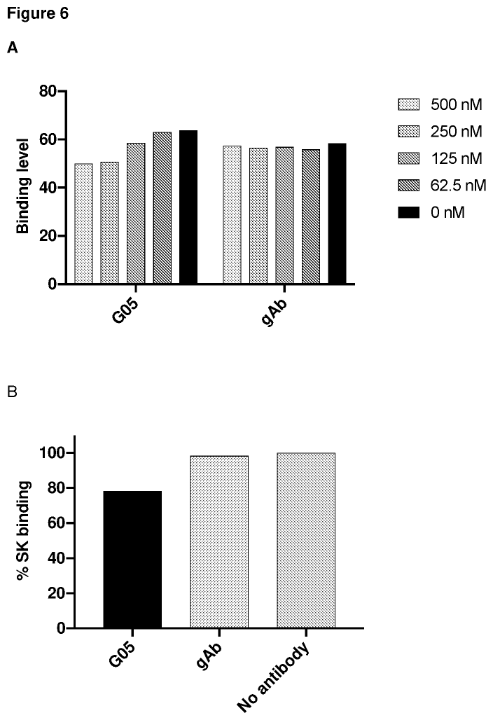

The result, shown in FIG. 6, indicates that G05 partially competes with streptokinase for plasminogen binding.

Inhibition of SK-Mediated Plasminogen Activation

Plasminogen activation by streptokinase was used to assess the ability of the antibody of the invention. 50 nM of plasminogen was activated with 5 nM of recombinant streptokinase at 37° C. The progress of plasminogen activation was monitored using 200 μM of plasmin fluorogenic substrate H-Ala-Phe-Lys-AMC (Bachem) in a Fluorstar Omega plate reader (BMG Labtech) via excitation and emission wavelengths of 355 nm and 460 nm, respectively. Individually, 0.5 μM of antibody was added at specific time points (t=0, t=60 min and t=160 min) during the process. HEPES-buffered saline was added in place of the antibody as a negative control.

The results in FIG. 7 show that the G05 antibody inhibits streptokinase mediated plasminogen activation effectively when it was added at t=0 and 10, and partially at t=60 min.

### Example 3: Binding of G05 and G11 to Recombinant Serine Protease Domain

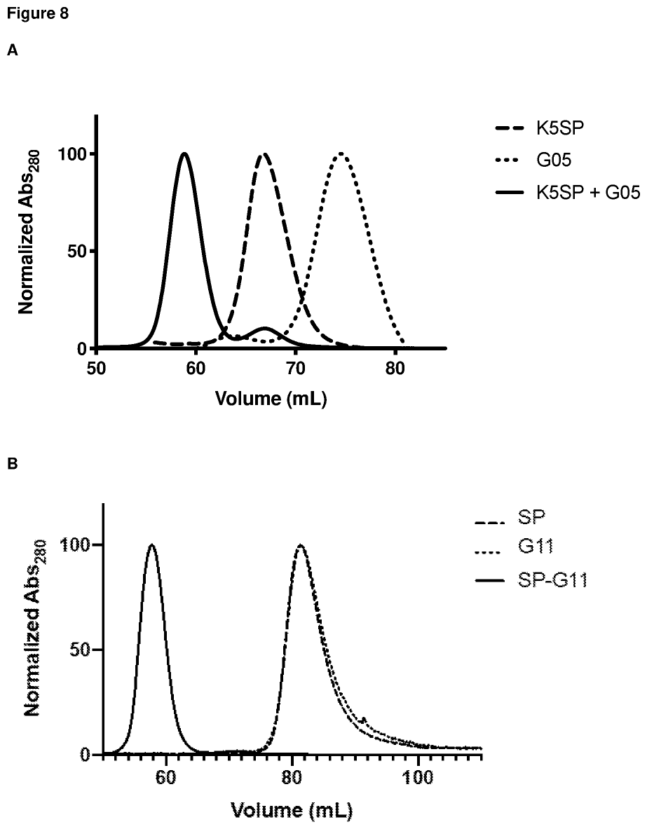

Binding of G05 to recombinant kringle 5-serine protease domain (KR5-SP) was assessed. G05 binds to and forms stable binary complex with KR5-SP that can be co-purified by size-exclusion chromatography. A Superdex 200 16/60 column (GE Healthcare) was used and the buffer was HEPES-buffered saline. As shown in FIG. 8A, the co-complex was eluted at 58.8 ml as a single peak. As a reference, the KR5-SP and G05 antibody was eluted at 66.7 ml and 74.6 ml, respectively, also as a single peak.

G11 binds to and forms stable binary complex with serine protease domain (SP) that can be co-purified by size-exclusion chromatography. A Superdex 75 16/60 column (GE Healthcare) was used and the buffer was HEPES-buffered saline. As shown in FIG. 8B, the co-complex was eluted at 58.0 ml as a single peak. As a reference, the SP and G11 were both eluted at 82 ml and dimer at 66.2 ml, respectively.

### Example 4: Crystal Structure of G05 Binding to Single Recombinant Kringle 5—Serine Protease Domain

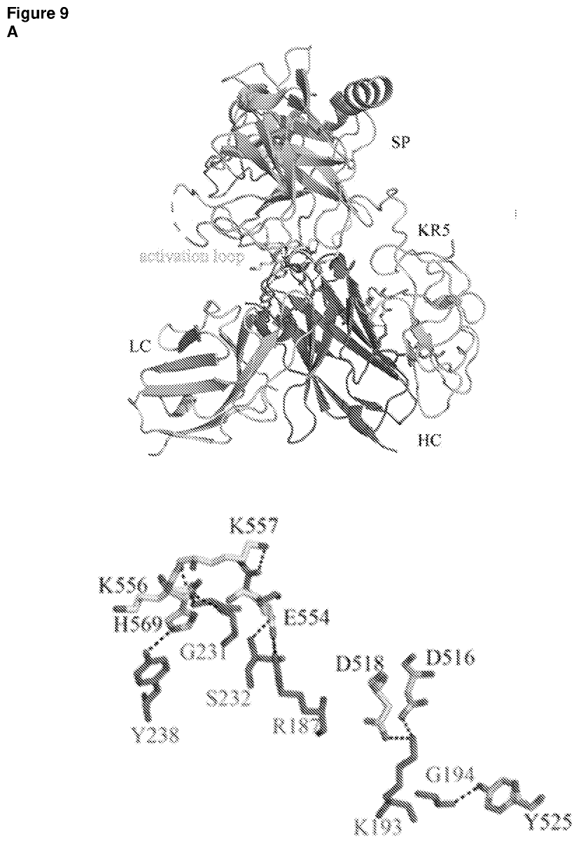

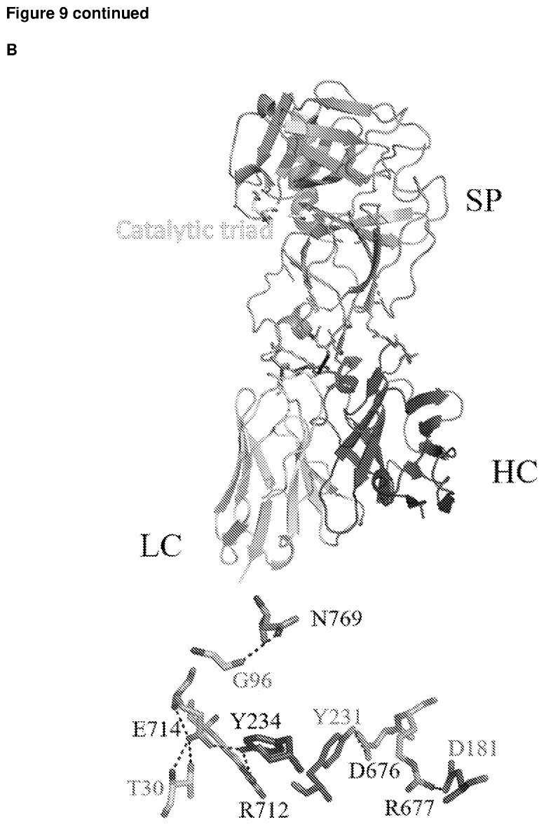

The purified G05/KR5-SP complexes were crystallized at 10 mg/ml in the presence of 0.1 M MES pH 6.5 and 10% (w/v) PEG 20,000 at 20° C. Crystals were flash cooled in liquid N2 in the presence of 20% (v/v) glycerol. A 2.0 Å dataset was collected at the Australian Synchrotron MX2 beamline using the EIGER X 16M pixel detector (Dectris Ltd, Switzerland also known as the ACRF detector). The crystal structure was solved using the program PHASER (CCP4) by molecular replacement, using the KR5 and SP domains from the structure of plasminogen (PDB ID 4DUR) and chicken single-chain fragment variable (PDB ID 4P48) as search models. Multiple rounds of modelling using COOT and refinement using PHENIX was performed. The final model shown in FIG. 9A was prepared using PyMOL (www.pymol.org/).

The G05-KR5-SP complex structure reveals that the antibody form polar interactions with the activation loop of plasminogen, such that it prevents plasmin generation by blocking plasminogen activators from cleaving the activation loop.

### Example 5: Crystal Structure of G11 Binding to Single Recombinant Serine Protease Domain

The purified G11/SP complexes were crystallized at 15 mg/ml in the presence of 0.1 M MES pH 6.0, 0.2 M lithium sulphate and 20% (w/v) PEG 4,000 at 20° C. Crystals were flash cooled in liquid N2 in the presence of 20% (v/v) glycerol. A 2.5 Å dataset was collected at the Australian Synchrotron MX2 beamline using the EIGER X 16M pixel detector (Dectris Ltd, Switzerland also known as the ACRF detector). The crystal structure was solved using the program PHASER (CCP4) by molecular replacement, using the SP domains from the structure of plasminogen (PDB ID 4DUR) and chicken single-chain fragment variable (PDB ID 4P48) as search models. Multiple rounds of modelling using COOT and refinement using PHENIX was performed. The final model shown (FIG. 9B) was prepared using PyMOL (www.pymol.org/).

The G11-SP complex structure reveals that the antibody forms polar interactions with the serine protease domain of plasminogen/plasmin, such that it prevents plasmin generation and activity via distorting the catalytic site and surrounding loops. This means that the enzyme is catalytically compromised.

### Example 6: Inhibition of Plasminogen Binding to GAS

An unactivatable mutant of plasminogen was used in the following experiments to determining binding to GAS.

By Flow Cytometry

GAS cells were grown in Todd-Hewitt (TH) broth from glycerol stock stab overnight at 37° C. in the shaker incubator. On the day of experiment, the culture was first sub-inoculated to OD₆₀₀ of 0.1 with fresh TH broth and allowed to grow in the shaker incubator as before. Early log-phase GAS at OD₆₀₀ of 0.35 was washed two times with phosphate buffered saline (PBS) supplemented with 2% plasminogen-depleted fetal calf serum (FCS) and resuspended in an equivalent volume. Recombinant plasminogen (22.2 nM) labelled with Alexa-Fluor 647 was incubated with 250 μl washed GAS cells (in) of for 30 mins at 4° C. Cells were then washed and immediately analysed by flow cytometry. To ensure that a homogenous cell population is analysed, single cells were gated to exclude larger entities, which were most likely streptococci chains. This is followed by the use of Syto-9 nucleic acid dye (ThermoFisher) to select for viable cell population.

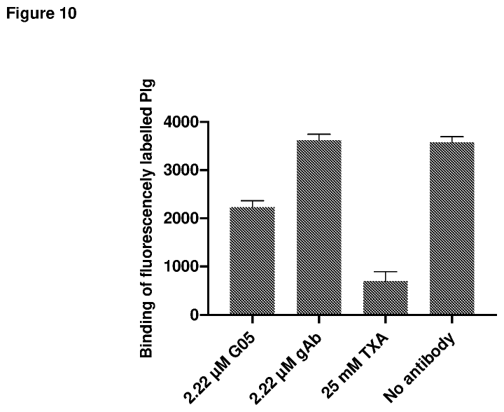

The result shown in FIG. 10 reveals that at 2.22 μM, G05 inhibits the binding of recombinant plasminogen to GAS by ˜40%. As a control, 25 mM tranexamic acid was used, which reduced recombinant plasminogen binding by around ˜80%. Naïve chicken antibody gAb, the negative control, showed no inhibition.

By SDS-Page

GAS culture was grown as above except with the OD₆₀₀ of 1.0. One ml of culture was used for each condition. Cells were washed twice, resuspended in 250 μl of PBS supplemented with 2% plasminogen-depleted FCS, 0.2 μM recombinant plasminogen was incubated with GAS together with 7.5 μM of G05, gAb, 13.5 mM TXA or buffer alone control. Following 30 mins incubation at 4° C., the supernatant (unbound fraction) was recovered. Cells were then washed twice as above, and the second wash was kept for SDS-PAGE. Bound plasminogen was eluted with 50 ul of 500 mM TXA, where cells were resuspended and spun down again. The resulting supernatant was collected as “bound fraction”. The unbound, wash and bound fractions together with recombinant plasminogen at 0.2 μM (Total) were separated on a 12% SDS-PAGE followed by a fluorescence scan using a Typhoon Gel Imaging System (Amersham) under Cy5 filter. Intensity of the bands were determined using a standard 2D gel analysis in ImageQuant TL software (GE Healthcare).

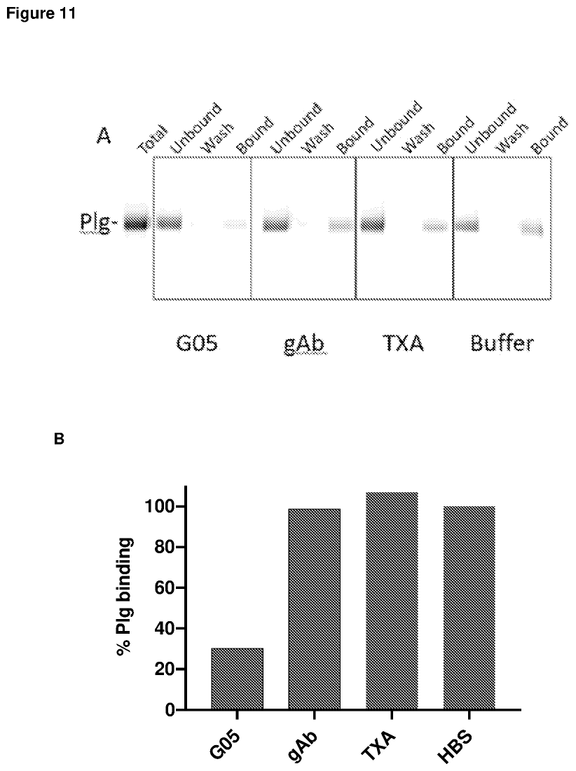

The result, shown in FIG. 11, indicates a 70% reduction in recombinant plasminogen binding to GAS by G05 compared to gAb and HEPES-buffered saline control. Interestingly, the positive control TXA did not show any inhibition. This result suggests that TXA, a plasmin inhibitor, likely does not inhibit GAS binding to plasminogen.

### Example 7: Inhibition of Plasminogen Activation on Group A Streptococcus (GAS) (Enzyme Assay)

GAS culture was grown as above but to the OD₆₀₀ of 1.0. 1 ml of culture was used for each sample. Cells were washed twice, and each sample was resuspended in 250 μl of PBS supplemented with 2% plasminogen-depleted FCS. 80 nM of plasminogen and 8 μM of G05/gAb or 13.5 mM TXA were mixed for 15 minutes at room temperature followed by incubation with the washed GAS cells for 1 hour at room temperature. After the 1-hour incubation, cells were washed twice and finally resuspended in 50 μl of PBS supplemented with 2% plasminogen-depleted FCS. 2 nM recombinant SK was added to half of the test samples to test whether SK needs to be replaced. 10 μl of the resuspended cells was added to a 100 μl reaction mixture buffered with 25 mM Tris, 150 mM NaCl, 0.05% Tween20 pH 7.4 and 200 μM fluorogenic substrate H-Ala-Phe-Lys-AMC (Bachem). Plasmin activity was measured at 37° C. in a Fluorstar Omega plate reader (BMG labtech) via excitation and emission wavelengths of 355 nm and 460 nm respectively.

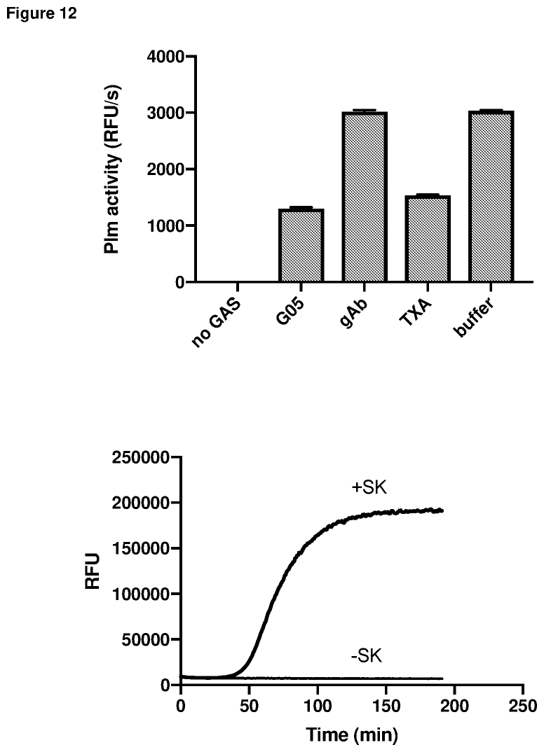

The result, shown in FIG. 12, shows that G05 inhibits plasminogen activation in the presence and absence of recombinant SK. As expected, gAb has no effect. TXA dissociates plasminogen from the cell surface, contributing to the increased activity seen with SK added (solution activation of Plg). FIG. 12 inset illustrates that in the absence of GAS cells, recombinant SK eventually activates Plg in solution.

### Example 8: hPLG is Recruited to the CDI-Damaged Gut Exacerbates Tissue Damage and Disease, and Anti-Plasminogen Antibody Reduces Disease Severity, Delays Onset of Disease and Increases Survival in Mice

Clostridium (now Clostridioides) difficile infects the gastrointestinal tract and causes severe tissue damage. A key driver of C. difficile infection (CDI) relates to the ability of this bacterium to form an inert, and highly robust spore form which allows survival of the bacterium in hostile environments. Spores initiate and transmit disease, and contribute to disease relapse—an event that occurs in up to 30% of patients. In the colon, spores germinate into vegetative cells that colonise the gut and produce up to three toxins, the large clostridial toxins TcdA and TcdB and the C. difficile transferase (CDT) toxin. These molecules act directly to damage and permeabilise colonic tissue through their effects on the actin cytoskeleton of host cells. C. difficile vegetative cells also produce spores, which are found at up to 1×10⁷ spores per gram of faeces in infected patients; these spores transmit disease and perpetuate the infection cycle.

Plasmin, the active form of the liver-produced zymogen plasminogen, is a serine protease that plays key roles in fibrinolysis, cell migration and tissue remodelling. Certain pathogens, including Group A Streptococcus and Bacillus anthracis, are known to exploit the fibrinolytic system by recruiting and activating plasminogen to the microbe surface. As a consequence of its ability to degrade host tissues, microbe-associated plasmin greatly enhances bacterial invasion of the host, thereby increasing disease severity during infection.

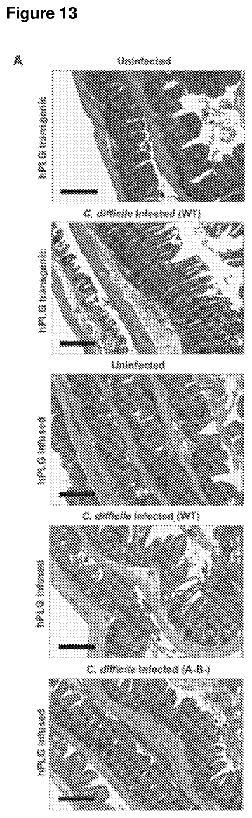

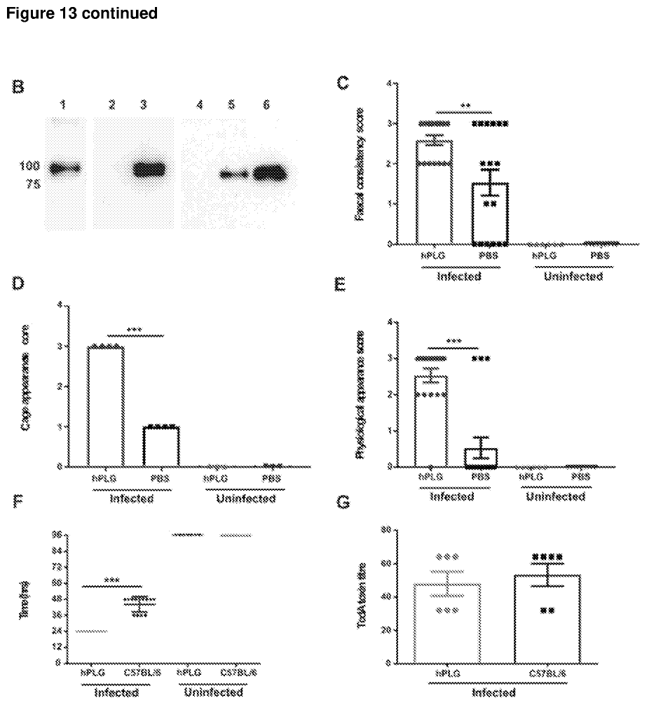

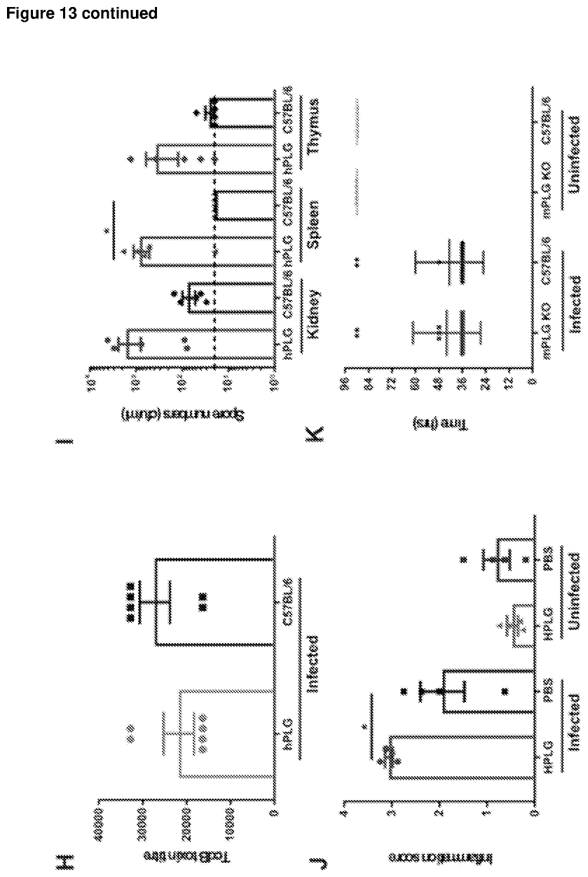

Under normal conditions plasminogen is in low abundance in the gastrointestinal tract. It was hypothesized, however, that as a consequence of its role in wound healing, the plasminogen system may be recruited to the site of infection where it may exacerbate or inhibit disease progression. To test this idea, mice infused with, or transgenic mice expressing, human plasminogen (hPLG) were infected with a toxigenic wild-type (WT) strain of C. difficile (M7404) or a mutant strain (DLL3121) that no longer produces the major toxins, TcdA and TcdB. WT-infected mice displayed epithelial damage, edema and inflammation in comparison to uninfected mice (FIG. 13A). It was found that hPLG levels in the cecum of these WT-infected mice were significantly elevated in comparison to all controls (FIG. 13B). In addition, slightly elevated levels of hPLG were detected in the DLL3121 infected mice compared to uninfected animals (FIG. 13B). These results suggest that CDI and tissue damage leads to an influx of hPLG into gastrointestinal tissue at the infection site. The action of the major toxins TcdA and TcdB appears to be the main driver of hPLG migration into the infection site, although other bacterial factors, such as the C. difficile binary toxin (CDT) or other virulence factors, are sufficient to mediate partial hPLG influx. Collectively, these results suggest that C. difficile infection and tissue damage leads to an influx of hPLG into gastrointestinal tissue at the infection site.

To determine if hPLG tissue influx influences infection outcomes, disease severity in wild type C57BL/6 mice was compared to that observed in transgenic mice that express hPLG, which were both infected with toxigenic C. difficile. Disease was greatly exacerbated when hPLG was present, with earlier disease onset seen in the hPLG-expressing mice as demonstrated by higher stool consistency scores (FIG. 13C), increased soiling of the nesting material (FIG. 13D), and poorer physiological conditions (FIG. 13E). The severity of the disease symptoms resulted in a significant reduction in survival time, with the hPLG transgenic mice requiring euthanasia 24 hours earlier than the WT mice (FIG. 13F). To ensure that disease exacerbation did not result from colonisation or toxin production differences between the groups of mice, feces were collected and analysed at 24 hours post-infection. TcdA and TcdB production was comparable across the two groups of mice (FIG. 13 G, H) and no discernible difference in the numbers of spores shed from the mice was detected, with all mice shedding 5×10⁶−1×10⁷ spores/g feces.

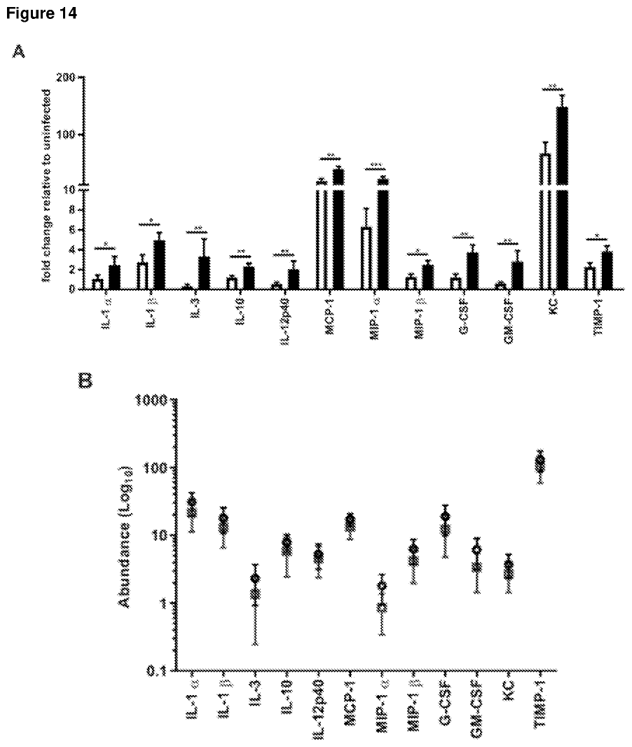

It was next investigated whether leakage of gut contents and dissemination of luminal contents to distant organs had taken place in the infected animals. It was reasoned that such an outcome may explain the observed disease exacerbation in hPLG mice. Accordingly, thymus, spleen and kidneys were harvested at 24 hours post-infection and examined for the presence of spores. Spores were detected in the kidney, spleen and thymus of the hPLG mice but not in C57BLJ6 control mice, except for very low numbers in the thymus (FIG. 131). An examination of cecal tissues harvested at the 24 hour post-infection time point revealed significantly more inflammation was evident in the hPLG mice compared to C57BLJ6 mice (FIG. 13J). These findings were supported by experiments performed using mice infused with hPLG, which also exhibited accelerated disease symptoms. The exacerbated disease seen in hPLG-expressing or infused mice was therefore reflected in the cecal pathology of the mice, suggesting that hPLG exacerbates gut inflammation during infection with toxigenic C. difficile which leads to more severe disease outcomes. The presence of hPLG in infected tissues also changed the host inflammatory profile, with significant increases in the production of pro-inflammatory cytokines and an anti-inflammatory cytokine detected in infected hPLG mice compared to infected PBS control mice (FIG. 14A). A quantitative proteomic analysis of infected versus uninfected cecal tissues collected from PBS and hPLG infused mice supported these findings (data not shown). Taken together, these data suggest that C. difficile is able to recruit hPLG during the course of infection, and that the presence of this protease enhances inflammation and disease severity, in turn CDI facilitating the translocation of C. difficile spores to extra-intestinal organs, the latter of which has not previously been reported.

Since the mice used in these infection experiments all produce murine plasminogen (mPLG) in addition to hPLG, the role of mPLG during infection was investigated by examining the disease outcomes after infection with toxigenic C. difficile of genetically modified mice that no longer express mPLG (mPLG KO) to unmodified C57BL/6 (WT) mice. No difference in disease progression or survival time (FIG. 13K) was observed between the two groups, suggesting that mPLG does not contribute to disease in this infection model.

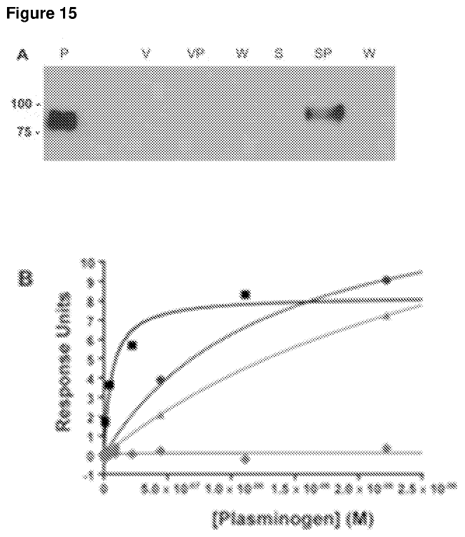

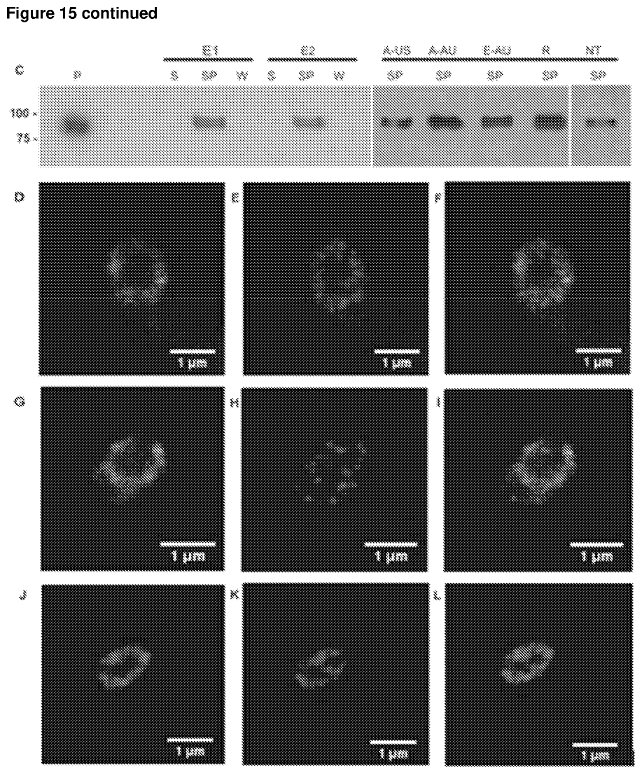

Since several diverse invasive pathogens, including bacteria and parasites, recruit plasminogen onto their surface to enable host invasion and to subvert the host immune response, it was examined if C. difficile has the same capability. Although it is the vegetative form of pathogenic bacteria that usually bind to plasminogen, in B. anthracis the spore form was found to bind plasminogen, albeit without consequence for disease severity or virulence. Unexpectedly, it was discovered that hPLG did not bind to C. difficile vegetative cells (FIG. 15A) but instead bound strongly to spores, regardless of the origin (human or animal), geographic location or toxigenic status of the bacterial strain (FIGS. 15A and C). Moreover, immunofluorescence (IF) and STED super resolution microscopy confirmed that hPLG (Alexa 488 for IF and Alexa 647 for STED) bound to spores produced under laboratory culture conditions (FIG. 15. D-F). Importantly, spores derived from C. difficile-infected patients (FIG. 15 G-I) or C. difficile-infected mice (FIG. 15J-L) were also found to have hPLG on their surface, with STED imaging showing that hPLG bound in clusters around the spore surface in all cases (FIG. 15 E, H, K). This result suggests that spores are naturally coated with hPLG within the context of the infected host.

Surface plasmon resonance confirmed that hPLG bound with high affinity to C. difficile spores (K_{D} of 13.4 nM±3.2 nM, Kₒₙ of 55,520 (±6953) M/s and K_{off} of 7.3×10⁻⁴ (±1.1×10⁻⁴) 1/s) (FIG. 3B). Crucially, no concentration dependent interaction was detected between spores and mPLG (FIG. 15B). These data support the finding that no difference in virulence is seen between WT versus murine plasminogen-deficient mice (FIG. 13K). C. difficile spores also bound to porcine and equine PLG (FIG. 15B) although with lesser affinity than to human PLG, which may suggest that these proteins also contribute to disease exacerbation in such hosts.

The conversion of PLG to its most active form, plasmin, is tightly regulated. hPLG is converted to plasmin by host factors such as urokinase plasminogen activator (uPA) and tissue plasminogen activator (tPA). Accordingly, PLG activation assays in the context of the spore-bound form were performed. Unbound or spore-bound hPLG was incubated with human or murine uPA (huPA and muPA) and human tPA (htPA). Both the mouse and human factors activated hPLG to plasmin in either the unbound or spore-bound state, suggesting that the spores have plasmin on their surface and can transport this active protease to any site that they occupy, within or outside the gut. This idea was supported by the proteomic analysis, which showed an increase in the amount of the mouse variants of the plasminogen-activating cascade only in infected hPLG mice (data not shown), indicating that hPLG is activated and functional in the gastrointestinal tract of these animals.

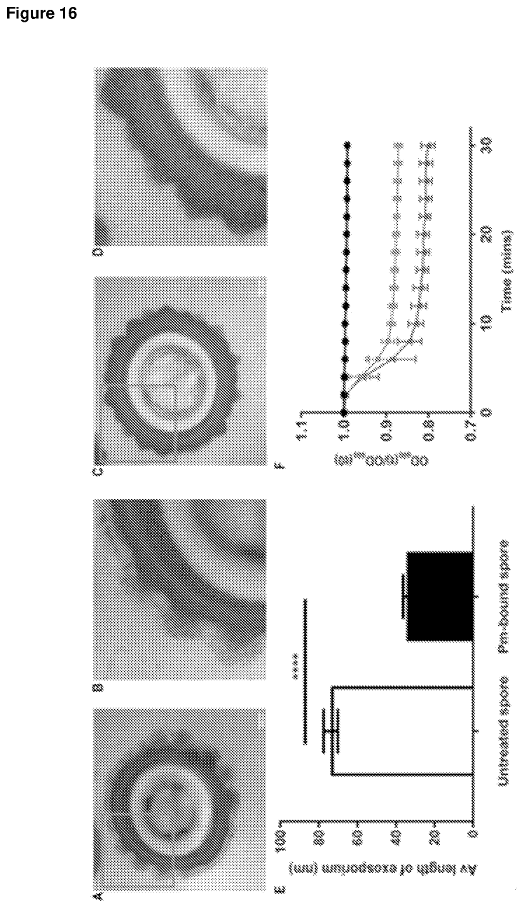

Since plasmin has broad substrate specificity, and to determine the functional outcome of plasminogen activation in the context of C. difficile spores, we investigated the spore structure to determine if it was altered, particularly on the surface. Strikingly, transmission electron microscopy showed that hPLG bound to spores and activated using huPA reduced the thickness of the exosporium, or the outermost spore layer, by half, from an average of 73.9 nm in untreated spores to 34.6 nm in hPLG-bound spores (FIG. 16A-E). Since germinants must traverse the exosporial layer before they can activate spore germination, the thickness and density of this layer may influence the efficiency of the germination process. Germination assays were therefore performed, in the presence of the known host-derived bile acid germinant, taurocholate, showing that the plasmin-bound spores germinated more rapidly than untreated spores (FIG. 16F). This result shows that binding and activation of hPLG directly changes the structural and functional properties of C. difficile spores, leading to faster germination in the presence of a physiologically relevant host germinant, which may contribute to the earlier disease onset and severe disease outcomes seen in the hPLG-mouse models described earlier. We also examined other functional consequences of spore-hPLG binding and activation with respect to host interactions and showed that plasmin-bound C. difficile spores cleave C3b and the extracellular matrix (ECM) protein, fibronectin, suggesting that spore-hPLG binding and activation to plasmin alters spore-host interactions. Plasmin-mediated modification of the surface of a bacterial cell, in this case a spore, has not been demonstrated before and brings a new understanding to how a pathogen has adapted to take advantage of the proteolytic activity of a host protein.

Collectively, these findings thus far suggest that the presence of hPLG appears to contribute to disease exacerbation in a multi-factorial manner, by inducing immune responses, degrading host tissues, and facilitating extra-intestinal spore dissemination. The latter event in particular may relate to disease recurrence.

To build on these findings, it was investigated whether targeting hPLG therapeutically may present an effective treatment for CDI. Previous studies have shown that the plasmin system is challenging to specifically inhibit, with small molecule plasmin inhibitors cross-reacting with other key proteases of the coagulation system. To overcome these problems, antibodies were developed that specifically target the plasminogen/plasmin system. Two antibodies were tested. One antibody, G05, was found to effectively inhibit the generation of active plasmin. Another antibody was found to effectively inhibit the activity of plasmin by binding to the catalytic triad of plasmin. The X-ray crystal structure of a scFv fragment of G05 in complex with a fragment (KR5-SP) of plasminogen (called KR5-SP, FIG. 9) was determined. The G05/KR5-SP complex structure revealed that the antibody specifically interacts with the activation loop of hPLG, thus preventing activators such as tPA and uPA from cleaving and activating the zymogen.

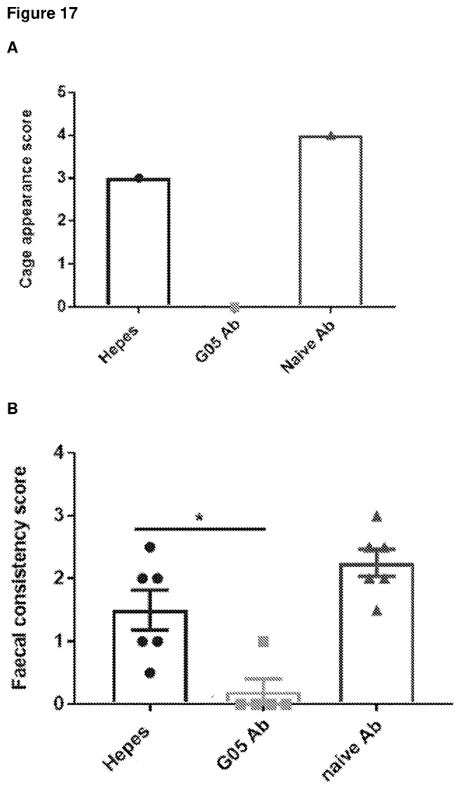

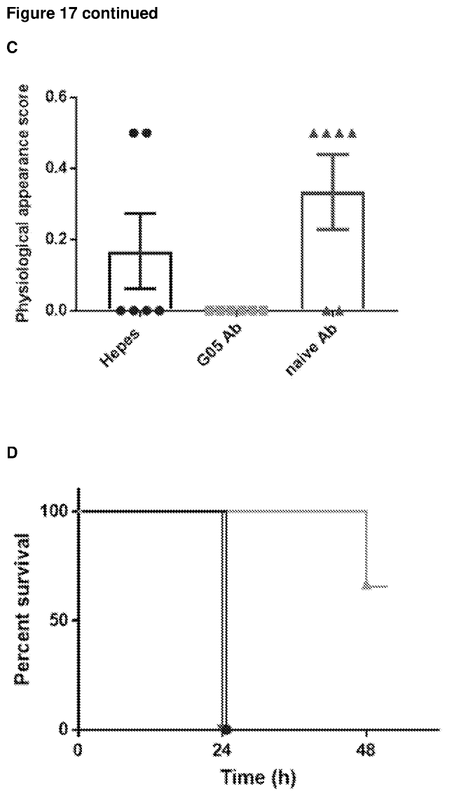

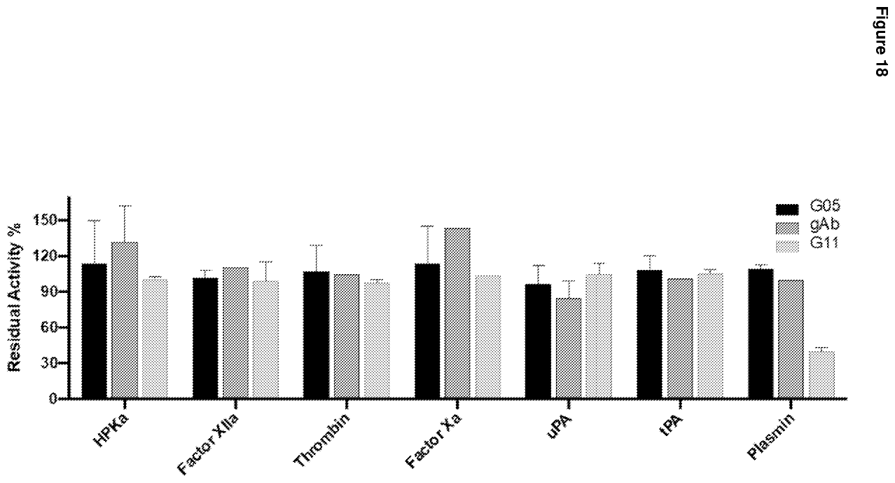

C57BL/6 mice that had been infused with hPLG infused and infected with C. difficile were injected intraperitoneally with HEPES buffer saline (HBS) alone, or a naïve antibody as control groups, or with antibody G05. Disease onset was not as rapid in the G05-treated group, with low scores detected at 24 h post-infection in both faecal consistency (FIG. 17B) and soiling of nesting material (FIG. 17A) when compared to the control groups. The physiological appearance between the G05-treated and control groups was also skewed toward a reduction in disease severity 24 h post-infection (FIG. 17C). Mouse survival beyond this time point was also striking, with G05-treated mice surviving for 24 h longer than control mice (FIG. 17D). These results indicate that administration of the plasmin generation inhibitory antibody G05 (i.e., an antibody that inhibits the activation of plasminogen to plasmin) protects against severe disease, and prolongs the survival of infected mice.

These data show that the dysregulation and increased amount of hPLG in the gastrointestinal tract following toxin damage changes the dynamics of C. difficile infection and allow for a new model for CDI progression to be proposed. In this model, toxin-mediated gut damage recruits hPLG to the damaged region. The aberrant localisation and activation of hPLG promotes further tissue damage through an inflammatory response that increases immune cell influx and degradation of host proteins. Once the infection is established hPLG may be recruited to both ingested spores and spores newly produced within the gut. Plasmin induces rapid spore germination, increasing the number of toxin-producing vegetative cells, further exacerbating host tissue damage, and enhancing disease severity. The plasmin bound spores also possess greater invasive potential and can be systemically spread throughout the host, which may increase the propensity for disease recurrence. In support of this model, antibody G05 which inhibits plasminogen activation successfully reduced disease severity. Since several gastrointestinal pathogens mediate disease through toxigenic effectors which compromise tissue integrity, and many other gastrointestinal disorders result in similar leaky gut effects, these results have broader implications for the understanding of the mechanisms underlying enteric diseases and raise the possibility that inhibition of human plasminogen or plasmin may be of broad therapeutic utility.

### Example 9: Administration of Antibody that Inhibits Plasminogen Activation is Useful for Reducing Bleeding

Wildtype 056B1/6 (WT) and mice harbouring a double mutation in the gene encoding plasminogen (Plg knockout Plg^{-/-}) were subjected to tail bleeding experiments.

200 μg of human plasminogen was injected intravenously into each animal. After 5 min, 50 μg of antibody (gAb or G05) was administered intravenously. Tail bleed experiments were performed by submerging the cut tails into warm saline for 20 min. The bleeding volume was measured by collecting red blood cells which were then suspended in a fixed volume of lysis buffer; OD₅₅₀ was recorded for each animal.

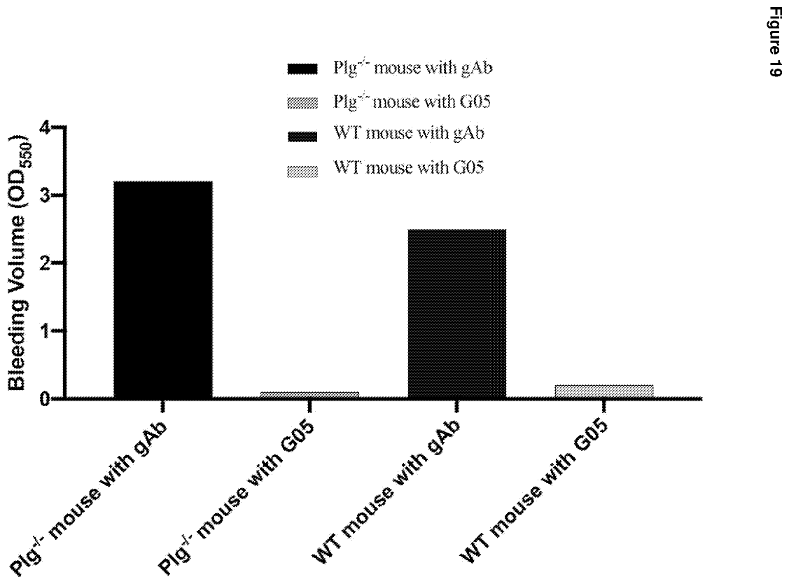

The data (shown in FIG. 19) show that the bleed volume for mice that received the G05 antibody was significantly reduced compared to mice that received naïve (gAb) antibody. These results indicate that an antibody that inhibits activation of plasminogen is useful for inhibiting bleeding in a model of trauma-induced bleeding.

### Example 10: G05 and G11 Inhibit Lysis of Synthetic Clots and Whole Blood Clots

Synthetic fibrin clots were formed by mixing 3 mg/ml fibrinogen (Banksia Scientific); 1 U of bovine thrombin (Jomar Life Research); and 10 nM of tPA (Boehringer Ingelheim), at 37° C. for 2 hours. Fibrinolysis was initiated by addition of 45 nM of plasminogen mixed with 0-3 μM G05; 0-2 μM G11; 0-90 nM α2AP; or 0-6.25 mM TXA, to the surface of the clot.

Fibrinolysis was monitored on a Nephelometer (BMG) at 37° C. for up to 10 hours. The time required to achieve 50% clot lysis was used for IC₅₀ calculation. The combination of G05 and G11 significantly inhibited clot lysis and was approximately 4 to 6-fold higher than when either antibody was used alone.

Whole blood clots were formed from human blood collected from healthy donors. Halo-shaped clots were generated by mixing whole blood with 15% of a mixture containing recombinant tissue factor supplemented with synthetic phospholipids (Dade Innovin, Siemens Germany) and 67 mM CaCl₂) in HBS at 1:4 ratio. The plate was sealed and incubated at 37° C. for 60 min before use.

Clot lysis was induced by the addition of tPA to 7 nM and antibodies or Plm inhibitors at the following concentrations: 0-312.5 nM B10; 0-1000 nM α2AP; 0-1000 nM aprotinin; 0-7.5 mM TXA. Clot lysis leads to an increase of turbidity and was monitored at OD₆₁₀ nm using a plate reader.

At high concentrations (e.g. up to 1,000 nM), α2AP and Aprotinin only partially inhibit clot lysis; TXA at a concentration up to 100 μM delayed clot lysis and total inhibition of clot lysis was observed at 300 μM and above (data not shown).

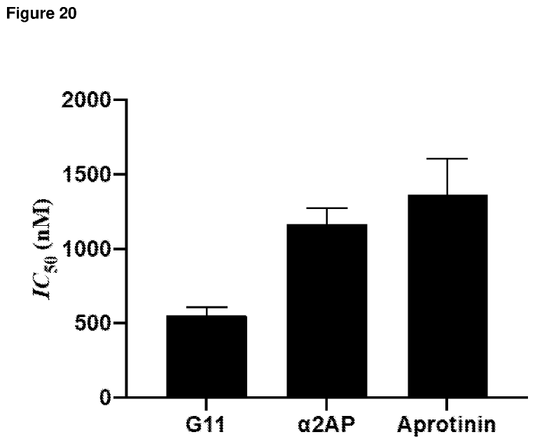

The time required to achieve 50% clot lysis was used for IC₅₀ calculation. The IC₅₀ value obtained for G11 was approximately 2-fold higher than for α2AP and approximately 2.5-fold higher than Aprotinin (FIG. 20).

Thus, in the context of a clot lysis assay which closely resembles a physiological system, antibody G11 is more effective than the physiological inhibitor of plasmin, α2AP, at inhibiting plasmin-induced lysis and more effective than a pharmacological inhibitor of plasmin, Aprotinin.

It will be understood that the invention disclosed and defined in this specification extends to all alternative combinations of two or more of the individual features mentioned or evident from the text or drawings. All of these different combinations constitute various alternative aspects of the invention.

1. An antigen binding protein comprising an antigen binding domain that binds to plasminogen, wherein the antigen binding protein reduces the activation of plasminogen.

2. The antigen binding protein of claim 1 wherein the antigen binding protein reduces or inhibits the activation of plasminogen by any one or more plasminogen activators.

3. The antigen binding protein of claim 2 wherein the plasminogen activator is any enzyme that can cleave the Arg₅₆₁-Val₅₆₂ bond of plasminogen (numbering as per human plasminogen), optionally wherein the plasminogen activators is a plasminogen-cleaving serine proteases selected from the group consisting of: the coagulation proteins factor IX, factor X, and prothrombin (factor II), protein C, chymotrypsin and trypsin, various leukocyte elastases, streptokinase (SK), staphylokinase, urokinase plasminogen activator (uPA), tissue plasminogen activator (tPA), and plasmin.

4. The antigen binding protein of any one of claims 1 to 3, wherein the antigen binding protein binds to the activation loop of plasminogen.

5. The antigen binding protein of any one of claims 1 to 4, wherein the antigen binding protein binds to a kringle domain of plasminogen, preferably to kringle domain 5.

6. The antigen binding protein of any one of claims 1 to 5, wherein the antigen binding protein also binds to kringle domain 5 and to the serine protease domain of plasminogen.

7. The antigen binding protein of any one of claims 1 to 6, wherein the antigen binding protein binds to region of plasminogen that comprises or consists of the amino acid sequence from between Arg₄₉₃ to His₅₆₉ of plasminogen (as set forth in SEQ ID NO: 65), preferably, wherein the protein binds to a region that comprises or consists of the amino acid sequence from between Lys₄₆₈ and His₅₆₉, more preferably wherein the activation loop comprises the sequence from Ala₅₄₃ to Arg₅₈₂ of plasminogen according to the numbering shown in SEQ ID NO: 65.

8. The antigen binding protein of any one of claims 1 to 7, wherein the antigen binding protein binds to a peptide comprising or consisting of or consisting essentially of the sequence as set forth in SEQ ID NO: 66, or a fragment thereof.

9. The antigen binding protein of any one of claims 1 to 8, wherein the antigen binding protein binds to one or more residues of a kringle 5 domain of plasminogen at a position, or position equivalent to, Arg₄₉₃, Ser₄₉₅, Ile₄₉₆, Asp₅₁₆, Gly₅₁₇, Asp₅₁₈, Val₅₁₉, Tyr₅₂₅, and Tyr₅₃₃, preferably, wherein the antigen binding protein binds to one or more residues of a kringle 5 domain of plasminogen at a position, or position equivalent to, those shown in Table 2.

10. The antigen binding protein of any one of claims 1 to 9, wherein the antigen binding protein binds to one or more residues of a serine protease domain of plasminogen at a position, or position equivalent to, Glu₅₅₄, Lys₅₅₆, Lys₅₅₇, His₅₆₉, and Asp₇₅₁, preferably wherein the antigen binding protein of the invention binds to one or more residues of a serine protease domain of plasminogen at a position, or position equivalent to, those shown in Table 2.

11. The antigen binding protein of any one of claims 1 to 8, wherein the antigen binding protein binds to one or more residues of a kringle 5 domain of plasminogen at a position, or position equivalent to, Lys₄₆₈, Arg₄₉₃, Ser₄₉₅, Ile₄₉₆, Asp₅₁₆, Gly₅₁₇, and Tyr₅₂₅, preferably wherein the antigen binding protein of the invention binds to one or more residues of a kringle 5 domain of plasminogen at a position, or position equivalent to, those shown in Table 3.

12. The antigen binding protein of any one of claim 1 to 8 or 11, wherein the antigen binding protein binds to one or more residues of a serine protease domain of plasminogen at a position, or position equivalent to, Glu₅₅₄, Lys₅₅₆, Lys₅₅₇, Arg₅₆₁, and His₅₆₉ preferably, wherein the antigen binding protein of the invention binds to one or more residues of a serine protease domain of plasminogen at a position, or position equivalent to, those shown in Table 3.

13. The antigen binding protein of any one of claims 1 to 12, wherein the interaction of a residue of the antigen binding protein with a residue of plasminogen is defined by x-ray crystallography and a contact distance analysis of 0 to 3.9 Å (inclusive).

14. The antigen binding protein of any one of claims 1 to 13, wherein the antigen binding protein binds to the same epitope on plasminogen as an antibody that comprises a VH domain comprising the amino acid sequence as set forth in SEQ ID NO: 8 and a VL domain comprising the amino acid sequence as set forth in SEQ ID NO: 7, wherein the antigen binding protein reduces or inhibits the activation of plasminogen.

15. The antigen binding protein of any one of claims 1 to 3, wherein the antigen binding protein binds to one or more residues of a serine protease domain of plasminogen at a position, or position equivalent to, Asp₆₇₆, Arg₆₇₇, Arg₇₁₂, Glu₇₁₄ and Asn₇₆₉, preferably wherein the antigen binding protein of the invention binds to one or more residues of a serine protease domain of plasminogen at a position, or position equivalent to, those shown in Table 4.

16. The antigen binding protein of any one of claim 1 to 3, 14 or 15, wherein the antigen binding protein binds to the same epitope on plasminogen as an antibody that comprises a VH domain comprising the amino acid sequence as set forth in SEQ ID NO: 40 and a VL domain comprising the amino acid sequence as set forth in SEQ ID NO: 39, wherein the antigen binding protein reduces or inhibits the activation of plasminogen.

17. The antigen binding protein of any one of claims 1 to 16, wherein the antigen binding protein binds to plasminogen and exhibits a K_{D} of less than 15 nM, less than 10 nM or equal to about 8 nM or less.

18. The antigen binding protein of any one of claims 1 to 17 wherein the antigen binding protein bind to plasminogen and exhibits a kₐ (M⁻¹s⁻¹) of greater than about 1×10⁴, greater than about 1×10⁴, or greater than or equal to about 1×10⁵ or greater than or equal to about 4×10⁵.

19. The antigen binding protein of any one of claims 1 to 18 wherein the antigen binding protein binds to plasminogen and exhibits a k_{d} (s⁻¹) of less than about 1×10⁻³, less than about 5×10⁻³, or less than or equal to about 2×10⁻³, or wherein the k_{d} is less than or equal to about 7×10⁻⁴.

20. The antigen binding protein of any one of claims 1 to 19 wherein the antigen binding protein inhibits streptokinase-, tPA- or uPA-mediated activation of plasminogen with an IC₅₀ of less than 500 nM, 300 nM, 250 nM, 200 nM, 150 nM, 100 nM, 80 nM, 60 nM or 50 nM, preferably about 40 nM.

21. The antigen binding protein of any one of claims 1 to 20 wherein the antigen binding protein binds to a peptide derived from the amino acid sequence set forth in SEQ ID NO: 65, optionally wherein the antigen binding protein binds to a peptide consisting of 4, 5, 7, 8, 9, 10 or more contiguous amino acid residues of the sequence of SEQ ID NO: 65.

22. The antigen binding protein of any one of claims 1 to 21 wherein the antigen binding protein binds to a peptide comprising, consisting essentially of or consisting of:
residues of 554 to 569 of SEQ ID NO: 65;
residues of 493 to 533 of SEQ ID NO: 65;
residues of 468 to 525 of SEQ ID NO: 65;
residues of 554 to 569 of SEQ ID NO: 65;
residues of 554 to 569 of SEQ ID NO: 65;
residues of 493 to 533 of SEQ ID NO: 65 and binds to a peptide comprising, consisting essentially of or consisting of residues of 554 to 569 of SEQ ID NO: 65;
residues of 468 to 525 of SEQ ID NO: 65, and binds to a peptide comprising, consisting essentially of or consisting of residues of 554 to 569 of SEQ ID NO: 65.

23. The antigen binding protein of any one of claims 1 to 22 wherein the antigen binding protein does not bind to a region of plasminogen or plasmin that only comprises the serine protease domain, preferably wherein the antigen binding protein does not bind to the catalytic triad of plasminogen or plasmin and/or does not significantly reduce the activity of plasmin.

24. The antigen binding protein of any one of claims 1 to 23 wherein the antigen binding protein does not significantly reduce the activity of any one or more of tPA, thrombin, trypsin, Factor Xa (FXa) and plasma kallikrein.

25. The antigen binding protein for binding to plasminogen of any one of claims 1 to 24, the antigen binding protein comprising:
FR1-CDR1-FR2-CDR2-FR3-CDR3-FR4
and
FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1, preferably wherein the framework regions have an amino acid sequence also as described in Table 1 below, including amino acid variation at particular residues which can be determined by aligning the various framework regions derived from each antibody.

26. The antigen binding protein of claim 25 wherein CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

27. The antigen binding protein of claim 25 or 26 wherein FR1-CDR1-FR2-CDR2-FR3-CDR3-FR4 and FR1a-CDR1a-FR2a-CDR2a-FR3a-CDR3a-FR4a are joined by a linker, optionally wherein the linker is a chemical, one or more amino acids, or a disulphide bond formed between two cysteine residues.

28. The antigen binding protein of any one of claims 1 to 27, comprising, consisting essentially of or consisting of an amino acid sequence of (in order of N to C terminus or C to N terminus):
SEQ ID NO: 7 and 8; or
SEQ ID NO: 39 and 40.

29. The antigen binding protein of any one of claims 1 to 28, comprising an antigen binding domain of an antibody, wherein the antigen binding domain binds to or specifically binds to plasminogen, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO:4, a CDR2 comprising a sequence at least about 80%, at least 85%, at least 90%, at least 92%, at least 95%, at least 97%, at least 99% identical to a sequence set in SEQ ID NO:5 and a CDR3 comprising a sequence at least about 80%, at least 85%, at least 90%, at least 92%, at least 95%, at least 97%, at least 99% identical to a sequence set forth in SEQ ID NO: 6;
(ii) a VH comprising a sequence at least about 95% or 96% or 97% or 98% or 99% identical to a sequence set forth in SEQ ID NO: 8;
(iii) a VL comprising a CDR1 comprising a sequence at least about 80%, at least 85%, at least 90%, at least 92%, at least 95%, at least 97%, at least 99% identical to a sequence set forth in SEQ ID NO: 1, a CDR2 comprising a sequence at least about 80%, at least 85%, at least 90%, at least 92%, at least 95%, at least 97%, at least 99% identical to a sequence set forth in SEQ ID NO: 2 and a CDR3 comprising a sequence at least about 80%, at least 85%, at least 90%, at least 92%, at least 95%, at least 97%, at least 99% identical to a sequence set forth in SEQ ID NO: 3;
(iv) a VL comprising a sequence at least about 95% identical to a sequence set forth in SEQ ID NO: 7;
(v) a VH comprising a CDR1 comprising a sequence set forth in SEQ ID NO: 4, a CDR2 comprising a sequence set forth between in SEQ ID NO: 5 and a CDR3 comprising a sequence set forth in SEQ ID NO: 6;
(vi) a VH comprising a sequence set forth in SEQ ID NO: 8;
(vii) a VL comprising a CDR1 comprising a sequence set SEQ ID NO: 1, a CDR2 comprising a sequence set forth in SEQ ID NO: 2 and a CDR3 comprising a sequence set forth in SEQ ID NO: 3;
(viii) a VL comprising a sequence set forth in SEQ ID NO: 7;
(ix) a VH comprising a CDR1 comprising a sequence set forth in SEQ ID NO: 4, a CDR2 comprising a sequence set forth between in SEQ ID NO: 5 and a CDR3 comprising a sequence set forth in SEQ ID NO: 6; and a VL comprising a CDR1 comprising a sequence set SEQ ID NO: 1, a CDR2 comprising a sequence set forth in SEQ ID NO: 2 and a CDR3 comprising a sequence set forth in SEQ ID NO: 3; or
(x) a VH comprising a sequence set forth in SEQ ID NO: 8 and a VL comprising a sequence set forth in SEQ ID NO: 7.

30. The antigen binding protein of claim 29 wherein the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:21, a FR2 comprising a sequence at least about 80%, at least 85%, at least 90%, at least 92%, at least 95%, at least 97%, at least 99% identical to a sequence set in SEQ ID NO:22, a FR3 comprising a sequence at least about 80%, at least 85%, at least 90%, at least 92%, at least 95%, at least 97%, at least 99% identical to a sequence set forth in SEQ ID NO: 23, and a FR4 comprising a sequence at least about 80%, at least 85%, at least 90%, at least 92%, at least 95%, at least 97%, at least 99% identical to a sequence set forth in SEQ ID NO: 24;
(ii) a VL comprising a FR1 comprising a sequence at least about 80%, at least 85%, at least 90%, at least 92%, at least 95%, at least 97%, at least 99% identical to a sequence set forth in SEQ ID NO: 17, a FR2 comprising a sequence at least about 80%, at least 85%, at least 90%, at least 92%, at least 95%, at least 97%, at least 99% identical to a sequence set forth in SEQ ID NO: 18, a FR3 comprising a sequence at least about 80%, at least 85%, at least 90%, at least 92%, at least 95%, at least 97%, at least 99% identical to a sequence set forth in SEQ ID NO: 19, and a FR4 comprising a sequence at least about 80%, at least 85%, at least 90%, at least 92%, at least 95%, at least 97%, at least 99% identical to a sequence set forth in SEQ ID NO: 20;
(iii) a VH comprising a FR1 comprising a sequence set forth in SEQ ID NO: 21, a FR2 comprising a sequence set forth between in SEQ ID NO: 22, a FR3 comprising a sequence set forth in SEQ ID NO: 23, and a FR4 comprising a sequence set forth in SEQ ID NO: 24;
(iv) a VL comprising a FR1 comprising a sequence set forth in SEQ ID NO: 17, a FR2 comprising a sequence set forth between in SEQ ID NO: 18, a FR3 comprising a sequence set forth in SEQ ID NO: 19, and a FR4 comprising a sequence set forth in SEQ ID NO: 20; or
(v) a VH comprising a FR1 comprising a sequence set forth in SEQ ID NO: 21, a FR2 comprising a sequence set forth between in SEQ ID NO: 22, a FR3 comprising a sequence set forth in SEQ ID NO: 23, and a FR4 comprising a sequence set forth in SEQ ID NO: 24; and a VL comprising a FR1 comprising a sequence set forth in SEQ ID NO: 17, a FR2 comprising a sequence set forth between in SEQ ID NO: 18, a FR3 comprising a sequence set forth in SEQ ID NO: 19, and a FR4 comprising a sequence set forth in SEQ ID NO: 20.

31. The antigen binding protein of any one of claims 1 to 28 comprising an antigen binding domain of an antibody, wherein the antigen binding domain binds to or specifically binds to plasminogen, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 36, a CDR2 comprising a sequence at least about 80%, at least 85%, at least 90%, at least 92%, at least 95%, at least 97%, at least 99% identical to a sequence set in SEQ ID NO: 37 and a CDR3 comprising a sequence at least about 80%, at least 85%, at least 90%, at least 92%, at least 95%, at least 97%, at least 99% identical to a sequence set forth in SEQ ID NO: 38;
(ii) a VH comprising a sequence at least about 95% or 96% or 97% or 98% or 99% identical to a sequence set forth in SEQ ID NO: 40;
(iii) a VL comprising a CDR1 comprising a sequence at least about 80%, at least 85%, at least 90%, at least 92%, at least 95%, at least 97%, at least 99% identical to a sequence set forth in SEQ ID NO: 33, a CDR2 comprising a sequence at least about 80%, at least 85%, at least 90%, at least 92%, at least 95%, at least 97%, at least 99% identical to a sequence set forth in SEQ ID NO: 34 and a CDR3 comprising a sequence at least about 80%, at least 85%, at least 90%, at least 92%, at least 95%, at least 97%, at least 99% identical to a sequence set forth in SEQ ID NO: 35;
(iv) a VL comprising a sequence at least about 95% identical to a sequence set forth in SEQ ID NO: 39;
(v) a VH comprising a CDR1 comprising a sequence set forth in SEQ ID NO: 36, a CDR2 comprising a sequence set forth between in SEQ ID NO: 37 and a CDR3 comprising a sequence set forth in SEQ ID NO: 38;
(vi) a VH comprising a sequence set forth in SEQ ID NO: 40;
(vii) a VL comprising a CDR1 comprising a sequence set SEQ ID NO: 33, a CDR2 comprising a sequence set forth in SEQ ID NO: 34 and a CDR3 comprising a sequence set forth in SEQ ID NO: 35;
(viii) a VL comprising a sequence set forth in SEQ ID NO: 39;
(ix) a VH comprising a CDR1 comprising a sequence set forth in SEQ ID NO: 36, a CDR2 comprising a sequence set forth between in SEQ ID NO: 37 and a CDR3 comprising a sequence set forth in SEQ ID NO: 38; and a VL comprising a CDR1 comprising a sequence set SEQ ID NO: 33, a CDR2 comprising a sequence set forth in SEQ ID NO: 34 and a CDR3 comprising a sequence set forth in SEQ ID NO: 35; or
(x) a VH comprising a sequence set forth in SEQ ID NO: 40 and a VL comprising a sequence set forth in SEQ ID NO: 39.

32. The antigen binding protein of claim 31 wherein the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO: 53, a FR2 comprising a sequence at least about 80%, at least 85%, at least 90%, at least 92%, at least 95%, at least 97%, at least 99% identical to a sequence set in SEQ ID NO: 54, a FR3 comprising a sequence at least about 80%, at least 85%, at least 90%, at least 92%, at least 95%, at least 97%, at least 99% identical to a sequence set forth in SEQ ID NO: 55, and a FR4 comprising a sequence at least about 80%, at least 85%, at least 90%, at least 92%, at least 95%, at least 97%, at least 99% identical to a sequence set forth in SEQ ID NO: 56;
(ii) a VL comprising a FR1 comprising a sequence at least about 80%, at least 85%, at least 90%, at least 92%, at least 95%, at least 97%, at least 99% identical to a sequence set forth in SEQ ID NO: 49, a FR2 comprising a sequence at least about 80%, at least 85%, at least 90%, at least 92%, at least 95%, at least 97%, at least 99% identical to a sequence set forth in SEQ ID NO: 50, a FR3 comprising a sequence at least about 80%, at least 85%, at least 90%, at least 92%, at least 95%, at least 97%, at least 99% identical to a sequence set forth in SEQ ID NO: 51, and a FR4 comprising a sequence at least about 80%, at least 85%, at least 90%, at least 92%, at least 95%, at least 97%, at least 99% identical to a sequence set forth in SEQ ID NO: 52;
(iii) a VH comprising a FR1 comprising a sequence set forth in SEQ ID NO: 53, a FR2 comprising a sequence set forth between in SEQ ID NO: 54, a FR3 comprising a sequence set forth in SEQ ID NO: 55, and a FR4 comprising a sequence set forth in SEQ ID NO: 56;
(iv) a VL comprising a FR1 comprising a sequence set forth in SEQ ID NO: 49, a FR2 comprising a sequence set forth between in SEQ ID NO: 50, a FR3 comprising a sequence set forth in SEQ ID NO: 51, and a FR4 comprising a sequence set forth in SEQ ID NO: 52; or
(v) a VH comprising a FR1 comprising a sequence set forth in SEQ ID NO: 53, a FR2 comprising a sequence set forth between in SEQ ID NO: 54, a FR3 comprising a sequence set forth in SEQ ID NO: 55, and a FR4 comprising a sequence set forth in SEQ ID NO: 56; and a VL comprising a FR1 comprising a sequence set forth in SEQ ID NO: 49, a FR2 comprising a sequence set forth between in SEQ ID NO: 50, a FR3 comprising a sequence set forth in SEQ ID NO: 51, and a FR4 comprising a sequence set forth in SEQ ID NO: 52.

33. The antigen binding protein of any one of claims 1 to 32, wherein the antigen binding protein is in the form of:
a single chain Fv fragment (scFv);
(ii) a dimeric scFv (di-scFv);
(iii) one of (i) or (ii) linked to a constant region of an antibody, Fc or a heavy chain constant domain (CH)2 and/or CH3; or
(iv) one of (i) or (ii) linked to a protein that binds to an immune effector cell.

34. The antigen binding protein of any one of claims 1 to 33, wherein the antigen binding protein is in the form of:
(i) a diabody;
(ii) a triabody;
(iii) a tetrabody;
(iv) a Fab;
(v) a F(ab′)2;
(vi) a Fv;
(vii) a bispecific antibody;
(viii) one of (i) to (vii) linked to a constant region of an antibody, Fc or a heavy chain constant domain (CH) 2 and/or CH3; or
(viv) one of (i) to (vii) linked to a protein that binds to an immune effector cell.

35. The antigen binding protein of any one of claims 1 to 34 wherein the antigen binding protein is an antibody or antigen binding fragment thereof, preferably wherein the antigen binding protein is a monoclonal antibody.

36. The antigen binding protein of any one of claims 1 to 35 wherein the antigen binding protein is a variable domain.

37. The antigen binding protein of any one of claims 1 to 36 wherein the protein is a plasminogen-binding antibody, the antibody comprising a light chain variable region and a heavy chain variable region,
wherein said light chain variable region comprises:
a CDR L1 as set forth in SEQ ID NO:1, a CDR L2 as set forth in SEQ ID NO:2 and a CDR L3 as set forth in SEQ ID NO:3; and
wherein said heavy chain variable region comprises:
a CDR H1 as set forth in SEQ ID NO:4, a CDR H2 as set forth in SEQ ID NO:5, and a CDR H3 as set forth in SEQ ID NO:6.

38. The antigen binding protein of claim 37, wherein the antibody comprises a light chain variable region that comprises a FR L1 as set forth in SEQ ID NO:17, FR L2 as set forth in SEQ ID NO:18, a FR L3 as set forth in SEQ ID NO:19 and a FR L4 as set forth in SEQ ID NO:20.

39. The antigen binding protein of claim 37 or 38 wherein the antibody comprises a heavy chain variable region that comprises a FR H1 as set forth in SEQ ID NO:21, FR H2 as set forth in SEQ ID NO:22, a FR H3 as set forth in SEQ ID NO:23 and a FR H4 as set forth in SEQ ID NO:24.

40. The antigen binding protein of claim 37 wherein the antibody comprises a light chain variable region that comprises the sequence of SEQ ID NO:7.

41. The antigen binding protein of claim 37 or 40, wherein the antibody comprises a heavy chain variable region that comprises the sequence of SEQ ID NO:8.

42. The antigen binding protein of any one of claims 1 to 36, wherein the protein is a plasminogen-binding antibody, the antibody comprising a light chain variable region and a heavy chain variable region,
wherein said light chain variable region comprises:
a CDR L1 as set forth in SEQ ID NO: 33, a CDR L2 as set forth in SEQ ID NO: 34 and a CDR L3 as set forth in SEQ ID NO: 35; and
wherein said heavy chain variable region comprises:
a CDR H1 as set forth in SEQ ID NO: 36, a CDR H2 as set forth in SEQ ID NO: 37, and a CDR H3 as set forth in SEQ ID NO: 38.

43. The antigen binding protein of claim 42 wherein the antibody comprises a light chain variable region that comprises a FR L1 as set forth in SEQ ID NO: 49, FR L2 as set forth in SEQ ID NO: 50, a FR L3 as set forth in SEQ ID NO: 51 and a FR L4 as set forth in SEQ ID NO: 52.

44. The antigen binding protein of claim 42 or 43 wherein the antibody comprises a heavy chain variable region that comprises a FR H1 as set forth in SEQ ID NO: 53, FR H2 as set forth in SEQ ID NO: 54, a FR H3 as set forth in SEQ ID NO: 55 and a FR H4 as set forth in SEQ ID NO: 56.

45. The antigen binding protein of claim 42 wherein the antibody comprises a light chain variable region that comprises the sequence of SEQ ID NO: 39.

46. The antigen binding protein of claim 42 or 45 wherein the antibody comprises a heavy chain variable region that comprises the sequence of SEQ ID NO: 40.

47. The antigen binding protein of any one of claims 1 to 46, wherein the antigen binding protein is a naked antibody.

48. A fusion protein comprising an antigen binding protein, immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody of any one of claims 1 to 46.

49. A conjugate in the form of an antigen binding protein, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody or fusion protein of any one of claim 1 to 46 or 48, conjugated to a label or a cytotoxic agent.

50. A nucleic acid encoding an antigen binding protein, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate of any one of claims 1 to 49.

51. A vector comprising the nucleic acid of claim 50.

52. A pharmaceutical composition comprising an antigen binding protein, of any one of claims 1 to 47, a fusion protein of claim 48 or a conjugate of claim 49 and a pharmaceutically acceptable carrier, diluent or excipient.

53. A method of restoring haemostasis in a subject who has suffered a trauma, a haemorrhage or is hemorrhaging comprising administering to a subject in need thereof, an antigen-binding protein of any one of claims 1 to 48, thereby restoring haemostasis in the subject.

54. A method for inhibiting fibrinolysis in a subject in need thereof comprising administering to a subject in need thereof, an antigen-binding protein of any one of claims 1 to 48, thereby inhibiting fibrinolysis in the subject, optionally wherein the subject has suffered a trauma, or has a haemorrhage due to surgery or child-birth.

55. A method of treating a bacterial infection in a subject, the method comprising administering an antigen binding protein of any one of claims 1 to 48 to the subject, thereby treating the bacterial infection in the subject.

56. A method for treating a condition associated with, or caused by, a bacterial infection in a subject, the method comprising administering to the subject an effective amount of the antigen binding protein of the invention, thereby treating the condition associated with, or caused by, a bacterial infection in the subject.

57. A method of reducing the severity of a bacterial infection in a subject, the method the method comprising administering an antigen binding protein of any one of claims 1 to 48 to the subject, thereby reducing the severity of the bacterial infection in the subject.

58. A method of treating or preventing a cancer in a subject, the method comprising administering an antigen binding protein of any one of claims 1 to 48 to the subject, thereby treating or preventing a cancer in the subject.

59. The method of claim 58, wherein the method comprises inhibiting, preventing or minimising spread or progression of a cancer, including inhibiting or preventing metastasis of cancer.

60. Use of a plasminogen-binding antigen binding protein of any one of claims 1 to 48, in the manufacture of a medicament for the restoration of haemostasis or inhibition of excessive plasminogen activation in a subject who has suffered a trauma, or requires restoration of haemostasis or inhibition of plasminogen activation following surgery or childbirth.

61. Use of a plasminogen-binding antigen binding protein of any one of claims 1 to 48, in the manufacture of a medicament for the treatment or prevention of a bacterial infection.

62. Use of an antigen binding protein of any one of claims 1 to 48, in the manufacture of a medicament for the treatment, prevention or reduction in severity of any condition or disease that is caused by or associated with a bacterial infection.

63. Use of an antigen binding protein of any one of claims 1 to 48, in the manufacture of a medicament for treating or preventing a cancer in a subject, optionally wherein the medicament is for inhibiting, preventing or minimising spread or progression of a cancer, including metastasis of a cancer.

64. A pharmaceutical composition of claim 52 for use in the restoration of haemostasis or inhibition of excessive plasminogen activation in a subject who has suffered a trauma, or requires restoration of haemostasis or inhibition of plasminogen activation following surgery or childbirth.

65. A pharmaceutical composition of claim 52 for use in the treatment or prevention of a bacterial infection.

66. A pharmaceutical composition of claim 52 for use in the treatment, prevention or reduction in severity of any condition or disease that is caused by or associated with a bacterial infection.

67. A pharmaceutical composition of claim 52 for use in treating or preventing a cancer in a subject, optionally wherein the composition is for inhibiting, preventing or minimising spread or progression of a cancer, including metastasis of a cancer.

68. The method of claim 55 or 56, use of claim 61 or 62, of composition of claim 65 or 66, wherein the bacterial infection is chronic or acute.

69. The method, use or composition of claim 68 wherein the infection is a bacterial infection.

70. The method, use or composition of claim 69 wherein the bacterial infection is caused by sporulating bacteria, optionally where the infection may be characterised by bacteria in the vegetative state, or in spore form.

71. The method, use or composition of claim 69 wherein the bacterial infection is with bacteria that are gram-positive, preferably gram-positive cocci.

72. The method, use or composition of claim 71, wherein the bacteria are from the family Streptococcaceae, preferably wherein the bacteria are from the genus Streptococcus, more preferably, wherein the bacteria are Group A streptococcus (GAS), including Streptococcus pyogenes.

73. The method, use or composition of claim 71, wherein the bacteria are from the family Staphylococcaceae, preferably wherein the bacteria are from the genus Staphylococcus, more preferably, the infection is an infection caused by the bacteria selected from the group consisting of: Staphylococcus aureus, including Methicillin-resistant Staphylococcus aureus (MRSA), Vancomycin-intermediate Staphylococcus aureus (VISA), and Vancomycin-resistant Staphylococcus aureus (VRSA).

74. The method, use or composition of claim 71 wherein the bacteria are from the family Peptostreptococcaceae, preferably wherein the bacteria are from the genus Clostridioides.

75. The method, use or composition of claim 71 wherein the bacterial infection is an infection with Clostridium difficile (also known as Clostridioides difficile).

76. The method, use or composition of claim 69, wherein the infection is with bacteria that are gram-negative, preferably from the order Enterobacteriales.

77. The method, use or composition of claim 76, wherein the bacteria are selected from the group consisting of: Yersinia pestis, Yersinia enterocolitica, Helicobacter pylori, E. coli, Salmonella sp., Pseudomonas sp., Shigella sp.


SEQUENCE LISTING (66 sequences)

SEQ ID NO: 1 - protein, 5 aa, Artificial Sequence
  G05 Antibody, LCDR1 (protein)
GRSSY

SEQ ID NO: 2 - protein, 3 aa, Artificial Sequence
  Antibody G05, LCDR2 (protein)
DNT

SEQ ID NO: 3 - protein, 12 aa, Artificial Sequence
  G05 Antibody, LCDR3 (protein)
GSADSSTTGIFG

SEQ ID NO: 4 - protein, 8 aa, Artificial Sequence
  G05 Antibody, HCDR1 (protein)
GFTFSSYA

SEQ ID NO: 5 - protein, 8 aa, Artificial Sequence
  G05 Antibody, HCDR2 (protein)
IDNIGSYT

SEQ ID NO: 6 - protein, 17 aa, Artificial Sequence
  G05 Antibody, HCDR3 (protein)
AKSAYGGSCCGPAYIDA

SEQ ID NO: 7 - protein, 107 aa, Artificial Sequence
  G05 Antibody, VL (protein)
QAALTQPSSVSANPGETVEITCSGGGRSSYYGWYQQKSPGSAPVTLIYDNTNRPSNIPSRFSGSKSGSTATLTITGVQGEDEAVYFCGSADSSTTGIFGAGTTLTVL

SEQ ID NO: 8 - protein, 124 aa, Artificial Sequence
  G05 Antibody, VH (protein)
AVTLDESGGGLQTPGRALSLVCKASGFTFSSYAMHWVRQAPGKGLEFVAGIDNIGSYTRYGAAVKGRVTISRDNGQSIVRLQLDNLRAEDTGTYYCAKSAYGGSCCGPAYIDAWGHGTEVIVSS

SEQ ID NO: 9 - DNA, 15 bp, Artificial Sequence
  G05 Antibody, LCDR1 (DNA)
ggacggtcatcctat

SEQ ID NO: 10 - DNA, 9 bp, Artificial Sequence
  G05 Antibody, LCDR2 (DNA)
gacaacaca

SEQ ID NO: 11 - DNA, 36 bp, Artificial Sequence
  G05 Antibody, LCDR3 (DNA)
ggcagcgccgatagctccaccacaggcatctttgga

SEQ ID NO: 12 - DNA, 24 bp, Artificial Sequence
  G05 Antibody, HCDR1 (DNA)
ggcttcacattttctagctatgcc

SEQ ID NO: 13 - DNA, 24 bp, Artificial Sequence
  G05 Antibody, HCDR2 (DNA)
atcgacaatatcggctcctacacc

SEQ ID NO: 14 - DNA, 51 bp, Artificial Sequence
  G05 Antibody, HCDR3 (DNA)
gcaaagagcgcctacggcggctcctgctgtggaccagcatatatcgacgca

SEQ ID NO: 15 - DNA, 321 bp, Artificial Sequence
  G05 Antibody, VL (DNA)
caggcggccctgactcagccctcttctgtctctgccaatccaggcgaaactgtcgaaattacttgttccggggggggacggtcatcctattacgggtggtatcagcagaagtctcccggcagcgcccctgtgaccctgatctatgacaacacaaataggccatccaacatcccctctcgcttctccggctctaagagcggctccaccgccacactgaccatcacaggagtgcagggagaggacgaggccgtgtacttctgcggcagcgccgatagctccaccacaggcatctttggagcaggcaccacactgaccgtgctg

SEQ ID NO: 16 - DNA, 372 bp, Artificial Sequence
  G05 Antibody, VH (DNA)
gccgtgaccctggatgagtccggaggaggcctccagacacctggccgggccctgagcctggtgtgcaaggccagcggcttcacattttctagctatgccatgcactgggtgaggcaggccccaggcaagggcctggagtttgtggccggcatcgacaatatcggctcctacaccagatatggagccgccgtgaagggaagggtgacaatctccagagataacggccagtctatcgtgcggctccagctggacaatctgagagccgaggataccggcacatactattgcgcaaagagcgcctacggcggctcctgctgtggaccagcatatatcgacgcatggggccacggcaccgaggtgatcgtgtcatcc

SEQ ID NO: 17 - protein, 25 aa, Artificial Sequence
  G05 Antibody, LFR1 (protein)
QAALTQPSSVSANPGETVEITCSGG

SEQ ID NO: 18 - protein, 18 aa, Artificial Sequence
  G05 Antibody, LFR2 (protein)
YGWYQQKSPGSAPVTLIY

SEQ ID NO: 19 - protein, 36 aa, Artificial Sequence
  G05 Antibody, LFR3 (protein)
NRPSNIPSRFSGSKSGSTATLTITGVQGEDEAVYFC

SEQ ID NO: 20 - protein, 8 aa, Artificial Sequence
  G05 Antibody, LFR4 (protein)
AGTTLTVL

SEQ ID NO: 21 - protein, 25 aa, Artificial Sequence
  G05 Antibody, HFR1 (protein)
AVTLDESGGGLQTPGRALSLVCKAS

SEQ ID NO: 22 - protein, 17 aa, Artificial Sequence
  G05 Antibody, HFR2 (protein)
MHWVRQAPGKGLEFVAG

SEQ ID NO: 23 - protein, 38 aa, Artificial Sequence
  G05 Antibody, HFR3 (protein)
RYGAAVKGRVTISRDNGQSIVRLQLDNLRAEDTGTYYC

SEQ ID NO: 24 - protein, 11 aa, Artificial Sequence
  G05 Antibody, HFR4 (protein)
WGHGTEVIVSS

SEQ ID NO: 25 - DNA, 75 bp, Artificial Sequence
  G05 Antibody, LFR1 (DNA)
caggcggccctgactcagccctcttctgtctctgccaatccaggcgaaactgtcgaaattacttgttccgggggg

SEQ ID NO: 26 - DNA, 54 bp, Artificial Sequence
  G05 Antibody, LFR2 (DNA)
tacgggtggtatcagcagaagtctcccggcagcgcccctgtgaccctgatctat

SEQ ID NO: 27 - DNA, 108 bp, Artificial Sequence
  G05 Antibody, LFR3 (DNA)
aataggccatccaacatcccctctcgcttctccggctctaagagcggctccaccgccacactgaccatcacaggagtgcagggagaggacgaggccgtgtacttctgc

SEQ ID NO: 28 - DNA, 24 bp, Artificial Sequence
  G05 Antibody, LFR4 (DNA)
gcaggcaccacactgaccgtgctg

SEQ ID NO: 29 - DNA, 75 bp, Artificial Sequence
  G05 Antibody, HFR1 (DNA)
gccgtgaccctggatgagtccggaggaggcctccagacacctggccgggccctgagcctggtgtgcaaggccagc

SEQ ID NO: 30 - DNA, 51 bp, Artificial Sequence
  G05 Antibody, HFR2 (DNA)
atgcactgggtgaggcaggccccaggcaagggcctggagtttgtggccggc

SEQ ID NO: 31 - DNA, 114 bp, Artificial Sequence
  G05 Antibody, HFR3 (DNA)
agatatggagccgccgtgaagggaagggtgacaatctccagagataacggccagtctatcgtgcggctccagctggacaatctgagagccgaggataccggcacatactattgc

SEQ ID NO: 32 - DNA, 33 bp, Artificial Sequence
  G05 Antibody, HFR4 (DNA)
tggggccacggcaccgaggtgatcgtgtcatcc

SEQ ID NO: 33 - protein, 3 aa, Artificial Sequence
  G11 Antibody, LCDR1 (protein)
GTY

SEQ ID NO: 34 - protein, 3 aa, Artificial Sequence
  G11 Antibody, LCDR2 (protein)
QNN

SEQ ID NO: 35 - protein, 13 aa, Artificial Sequence
  G11 Antibody, LCDR3 (protein)
GGYDSSAGYATFG

SEQ ID NO: 36 - protein, 8 aa, Artificial Sequence
  G11 Antibody, HCDR1 (protein)
GFSISSYS

SEQ ID NO: 37 - protein, 8 aa, Artificial Sequence
  G11 Antibody, HCDR2 (protein)
ISSDGSDT

SEQ ID NO: 38 - protein, 18 aa, Artificial Sequence
  G11 Antibody, HCDR3 (protein)
AKPTGGYYTWYETGSIDT

SEQ ID NO: 39 - protein, 106 aa, Artificial Sequence
  G11 Antibody, VL (protein)
QAALTQPSSVSANPGEIVEITCSGGGTYYGWYQQKSPGSAPVTMIYQNNQRPSNIPSRFSGSGSGPANTLTITGVRAEDEAVYYCGGYDSSAGYATFGAGTTLTVL

SEQ ID NO: 40 - protein, 125 aa, Artificial Sequence
  G11 Antibody, VH (protein)
AVTLDESGGGLQTPGGGLSLVCKASGFSISSYSMYWVRQAPGKGLEFVASISSDGSDTSYGSAVKGRATISRDNGQSTVRLQLNYLRAEDTGTYFCAKPTGGYYTWYETGSIDTWGHGTEVIVSS

SEQ ID NO: 41 - DNA, 9 bp, Artificial Sequence
  G11 Antibody, LCDR1 (DNA)
ggcacctac

SEQ ID NO: 42 - DNA, 9 bp, Artificial Sequence
  G11 Antibody, LCDR2 (DNA)
cagaacaat

SEQ ID NO: 43 - DNA, 39 bp, Artificial Sequence
  G11 Antibody, LCDR3 (DNA)
ggcggctacgatagctccgccggctatgccacatttggc

SEQ ID NO: 44 - DNA, 24 bp, Artificial Sequence
  G11 Antibody, HCDR1 (DNA)
ggcttcagcatctctagctactcc

SEQ ID NO: 45 - DNA, 24 bp, Artificial Sequence
  G11 Antibody, HCDR2 (DNA)
atctcctctgacggctccgataca

SEQ ID NO: 46 - DNA, 54 bp, Artificial Sequence
  G11 Antibody, HCDR3 (DNA)
gccaagcccaccggcggctactatacatggtacgagacaggctccatcgataca

SEQ ID NO: 47 - DNA, 318 bp, Artificial Sequence
  G11 Antibody, VL (DNA)
caggcggccctgactcagccttcttctgtctccgccaaccccggcgagatcgtggagatcacatgctctggcggcggcacctactatggctggtatcagcagaagagcccaggctccgcccctgtgaccatgatctatcagaacaatcagaggccatccaacatcccctctcgcttcagcggctccggctctggacctgcaaataccctgacaatcaccggcgtgagggcagaggacgaggccgtgtactattgcggcggctacgatagctccgccggctatgccacatttggcgcaggcaccacactgaccgtgctg

SEQ ID NO: 48 - DNA, 375 bp, Artificial Sequence
  G11 Antibody, VH (DNA)
gccgtgacactggacgagagcggaggaggcctgcagacccctggaggaggcctgagcctggtgtgcaaggcctctggcttcagcatctctagctactccatgtattgggtgcggcaggcaccaggcaagggcctggagtttgtggccagcatctcctctgacggctccgatacatcttacggcagcgccgtgaagggaagggcaacaatcagcagagacaacggccagtccaccgtgcggctgcagctgaattacctgagagccgaggatacaggcacctatttctgtgccaagcccaccggcggctactatacatggtacgagacaggctccatcgatacatggggccacggcaccgaagtgatcgtctcatca

SEQ ID NO: 49 - protein, 25 aa, Artificial Sequence
  G11 Antibody, LFR1 (protein)
QAALTQPSSVSANPGEIVEITCSGG

SEQ ID NO: 50 - protein, 18 aa, Artificial Sequence
  G11 Antibody, LFR2 (protein)
YGWYQQKSPGSAPVTMIY

SEQ ID NO: 51 - protein, 36 aa, Artificial Sequence
  G11 Antibody, LFR3 (protein)
QRPSNIPSRFSGSGSGPANTLTITGVRAEDEAVYYC

SEQ ID NO: 52 - protein, 8 aa, Artificial Sequence
  G11 Antibody, LFR4 (protein)
AGTTLTVL

SEQ ID NO: 53 - protein, 25 aa, Artificial Sequence
  G11 Antibody, HFR1 (protein)
AVTLDESGGGLQTPGGGLSLVCKAS

SEQ ID NO: 54 - protein, 17 aa, Artificial Sequence
  G11 Antibody, HFR2 (protein)
MYWVRQAPGKGLEFVAS

SEQ ID NO: 55 - protein, 38 aa, Artificial Sequence
  G11 Antibody, HFR3 (protein)
SYGSAVKGRATISRDNGQSTVRLQLNYLRAEDTGTYFC

SEQ ID NO: 56 - protein, 11 aa, Artificial Sequence
  G11 Antibody, HFR4 (protein)
WGHGTEVIVSS

SEQ ID NO: 57 - DNA, 75 bp, Artificial Sequence
  G11 Antibody, LFR1 (DNA)
caggcggccctgactcagccttcttctgtctccgccaaccccggcgagatcgtggagatcacatgctctggcggc

SEQ ID NO: 58 - DNA, 54 bp, Artificial Sequence
  G11 Antibody, LFR2 (DNA)
tatggctggtatcagcagaagagcccaggctccgcccctgtgaccatgatctat

SEQ ID NO: 59 - DNA, 108 bp, Artificial Sequence
  G11 Antibody, LFR3 (DNA)
cagaggccatccaacatcccctctcgcttcagcggctccggctctggacctgcaaataccctgacaatcaccggcgtgagggcagaggacgaggccgtgtactattgc

SEQ ID NO: 60 - DNA, 24 bp, Artificial Sequence
  G11 Antibody, LFR4 (DNA)
gcaggcaccacactgaccgtgctg

SEQ ID NO: 61 - DNA, 75 bp, Artificial Sequence
  G11 Antibody, HFR1 (DNA)
gccgtgacactggacgagagcggaggaggcctgcagacccctggaggaggcctgagcctggtgtgcaaggcctct

SEQ ID NO: 62 - DNA, 51 bp, Artificial Sequence
  G11 Antibody, HFR2 (DNA)
atgtattgggtgcggcaggcaccaggcaagggcctggagtttgtggccagc

SEQ ID NO: 63 - DNA, 114 bp, Artificial Sequence
  G11 Antibody, HFR3 (DNA)
tcttacggcagcgccgtgaagggaagggcaacaatcagcagagacaacggccagtccaccgtgcggctgcagctgaattacctgagagccgaggatacaggcacctatttctgt

SEQ ID NO: 64 - DNA, 33 bp, Artificial Sequence
  G11 Antibody, HFR4 (DNA)
tggggccacggcaccgaagtgatcgtctcatca

SEQ ID NO: 65 - protein, 791 aa, Homo sapiens
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 66 - protein, 16 aa, Homo sapiens
EPKKCPGRVVGGCVAH